(19) United States
(12) Patent Application Publication
(10) Pub. No.: US 2023/0357168 A1
(43) Pub. Date: Nov. 9, 2023

(54) CARBOBICYCLIC NUCLEOSIDES FOR TREATMENT OF VIRAL INFECTION

(71) Applicant: The Chinese University of Hong Kong, Shatin (CN)

(72) Inventors: Wai-Lung NG, Shatin (CN); Mao-Yun LYU, Shatin (CN); Stephan SCHEEFF, Mongkok (HK)

(21) Appl. No.: 18/134,770

(22) Filed: Apr. 14, 2023

Related U.S. Application Data
(60) Provisional application No. 63/331,670, filed on Apr. 15, 2022.

(51) Int. Cl. C07D 249/10; A61P 31/14; C07D 473/34; C07D 473/30; C07D 239/54; C07D 239/47; C07D 487/04

(52) U.S. Cl. CPC C07D 249/10; A61P 31/14; C07D 473/34; C07D 473/30; C07D 239/54; C07D 239/47; C07D 487/04

(57) ABSTRACT

Carbobicyclic nucleoside compounds useful for the treatment of coronavirus disease 2019 (COVID-19), RSV and other viral infections are provided. The carbobicyclic nucleoside compound may be a compound as set forth herein. Pharmaceutical formulations useful for treatment of COVID-19 and RSV are also described.

### CROSS-REFERENCE TO RELATED APPLICATION(S)

The present application claims priority to U.S. Provisional Patent Application No. 63/331,670, filed on Apr. 15, 2022, the disclosure of which is incorporated herein by reference in its entirety for all purposes.

### BACKGROUND OF THE INVENTION

The novel pneumonia-causing virus SARS-CoV-2 has been declared a global emergency by the World Health Organization (WHO), and is expected to spread continuously.

As of early February 2022, there have been more than 380,000,000 confirmed cases of COVID-19, including over 5,600,000 deaths, reported to the WHO. The search for an efficacious antiviral agent to mitigate the epidemic is ongoing. It has been reported that existing nucleoside analogues such as remdesivir are effective against SARS-CoV-2. Remdesivir (a ribose-based nucleoside) is susceptible to the metabolic pathways of the host, however, rendering it prone to degradation in vivo. In addition, the target enzyme of remdesivir, RNA-dependent RNA polymerase (RdRp), has the possibility to develop resistance to ribose-based nucleoside analogues. This is known, for example, in the case of ribavirin-resistant severe acute respiratory syndrome coronavirus (SARS-CoV).

### BRIEF SUMMARY OF THE INVENTION

In one aspect, the disclosure provides a compound according to the following formula:

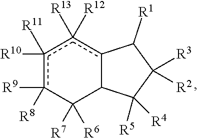

a pharmaceutically acceptable salt thereof, or an enantiomer thereof, wherein: R¹ is nitrogen-containing monocyclic or bicyclic heterocyclyl; each of R², R³, R⁴, R⁵, R⁶, and R⁷ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, optionally substituted carboxylate, or OPₓ; each of R⁸, R⁹, R¹⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, OPₓ, or absent; Pₓ is monophosphate, diphosphate, triphosphate, C2-6 acyl, or a phosphate prodrug moiety; and each dashed line represents a single or double bond, provided that no more than one double bond is present.

In some embodiments, the compound, or a pharmaceutically acceptable salt thereof, comprises a structure according to the following formula:

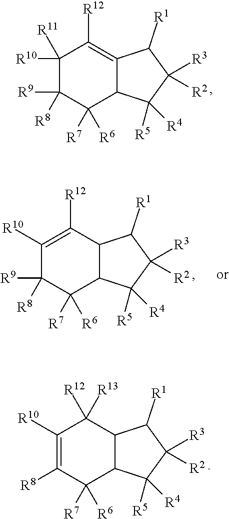

In particular embodiments, the compound, or a pharmaceutically acceptable salt thereof, comprises a structure according to the following formula:

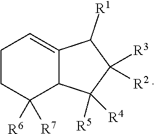

In some embodiments, the compound, or a pharmaceutically acceptable salt thereof, comprises a structure according to the following formula:

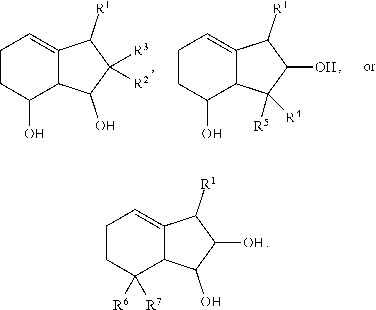

Specifically, the compound, or a pharmaceutically acceptable salt thereof, comprises a structure according to the following formula:

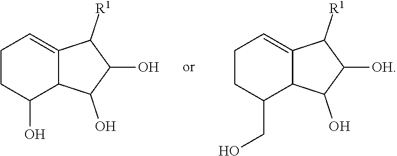

More specifically, the compound, or a pharmaceutically acceptable salt thereof, comprises a structure according to the following formula:

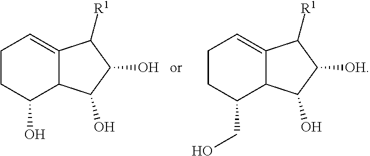

In some embodiments, the compound, or a pharmaceutically acceptable salt thereof, comprises a structure according to the following formula

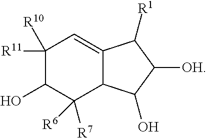

In some embodiments, the compound, or a pharmaceutically acceptable salt thereof, comprises a structure according to the following formula:

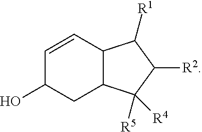

In some embodiments, a compound, or a pharmaceutically acceptable salt thereof, comprises a structure according to the following formula:

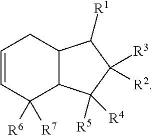

In particular embodiments, the compound, or a pharmaceutically acceptable salt thereof, comprises a structure according to the following formula:

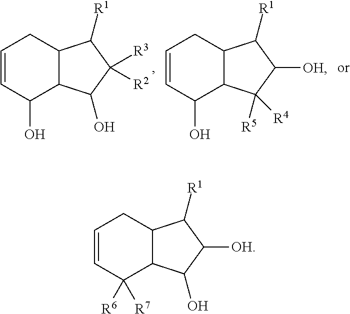

In some embodiments, a compound, or a pharmaceutically acceptable salt thereof, comprises a structure according to the following formula:

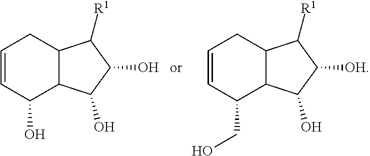

In some embodiments of the formulas described above, each of R², R³, R⁴, and R⁵ is, independently, H, deuterium, hydroxyl, Cl, F, CN, Me, Et, (C₁₋₆)OH, ORₓ, (C₁₋₆)COORₓ, or (C₁₋₆)CON(Rₓ)₂; each of R⁶ and R⁷ is, independently, H, deuterium, hydroxyl, Cl, F, CN, Me, Et, (C₁₋₆)OH, ORₓ, (C₁₋₆)COORₓ, (C₁₋₆)CON(Rₓ)₂, optionally substituted carboxylate, or OPₓ; Pₓ is monophosphate, diphosphate, triphosphate, C₂₋₆ acyl, or a phosphate prodrug moiety; and each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy.

In some embodiments of the disclosure, the compound, or a pharmaceutically acceptable salt thereof, comprises a structure according to the following formula:

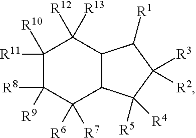

wherein: R¹ is optionally substituted nitrogen-containing monocyclic or bicyclic heterocyclyl; each of R², R³, R⁴, R⁵, R⁶, R⁷, R⁸, R⁹, R¹⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy, optionally substituted carboxylate, or OPₓ, wherein Pₓ is monophosphate, diphosphate, triphosphate, C2-6 acyl, or a phosphate prodrug moiety. In some embodiments, the compound, or a pharmaceutically acceptable salt thereof, comprises a structure according to the following formula:

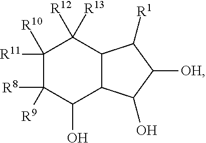

wherein: R¹ is optionally substituted nitrogen-containing monocyclic or bicyclic heterocyclyl; each of R¹⁰, R¹¹, R¹², and R¹³ is, independently, H, deuterium, hydroxyl, Cl, F, CN, Me, Et, (C₁₋₆)OH, ORₓ, (C₁₋₆)COORₓ, (C₁₋₆)CON(Rₓ)₂, epoxide, or cyclopropane; each of R⁸, R⁹ is, independently, H, deuterium, hydroxyl, Cl, F, CN, Me, Et, (C₁₋₆)OH, ORₓ, (C₁₋₆)COORₓ, (C₁₋₆)CON(Rₓ)₂, epoxide, cyclopropane, or OPₓ, Pₓ is monophosphate, diphosphate, triphosphate, C2-6 acyl, or a phosphate prodrug moiety; each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy. In particular embodiments, the compound, or a pharmaceutically acceptable salt thereof, comprises a structure according to the following formula:

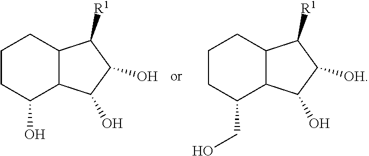

In any of the formulas described above, in some embodiments, R¹ is selected from the group consisting of adenine, guanine, cytosine, N⁴-hydroxycytosine, thymine, uracil, pyrrolo[2,1-f][1,2,4]triazin-4-amine, and 1,2,4-triazole-3-carboxamide. In some embodiments, R¹ is selected from the group consisting of adenine, cytosine, and N⁴-hydroxycytosine.

In some embodiments of the formulas described herein, R¹ is

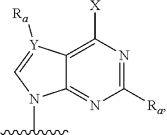

wherein each of X and Rₐ is, independently, H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy; and Y is N or C. In some embodiments, each of X and Rₐ is, independently, H, deuterium, hydroxyl, Cl, F, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy; and Y is N or C. In particular embodiments, R¹ is:

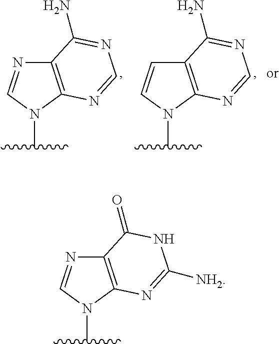

In some embodiments of the formulas described herein, R¹ is

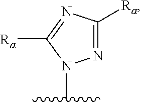

wherein each of Rₐ is, independently, H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy. In some embodiments, R¹ is

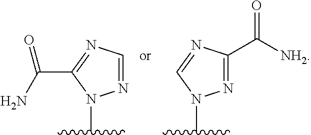 [SMILES: *n1ncnc1C(N)=O ; *n1cnc(C(N)=O)n1]

In some embodiments of the formulas described herein, R¹ is

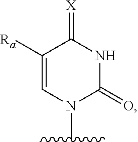

wherein Rₐ is H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ; and X is O, NRₓ, or NORₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy. In some embodiments, R¹ is

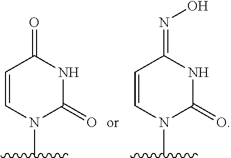

In some embodiments of the formulas described herein, Pₓ is a phosphate prodrug moiety, e.g., a phosphate prodrug moiety is selected from the group consisting of a phosphonate, carbonyloxymethyl phosphate ester, an S-acyl-2-thioethyl phosphate ester, an S-[(2-hydroxethyl)]sulfidyl]-thioethyl phosphate ester, a cyclosaligenyl phosphate diester, a cyclic 1-aryl-1,3-propanyl phosphate diester, an alkoxyalkyl phosphate ester, an aryloxy phosphoramidate, an aryloxy phosphonamidate, an amino acid phosphoramidate, an amino acid phosphonamidate, and a methylaryl haloalkyl phosphoramidate.

In another aspect, the disclosure also provides a compound comprising one of the following formulas, or a pharmaceutically acceptable salt thereof.

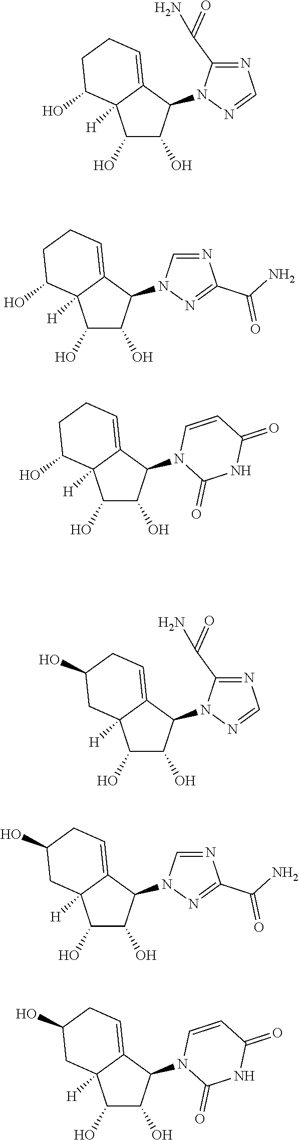 [SMILES: NC(=O)c1ncnn1[C@@H]1C2=CCC[C@@H](O)[C@@H]2[C@@H](O)[C@H]1O ; NC(=O)c1ncn([C@@H]2C3=CCC[C@@H](O)[C@@H]3[C@@H](O)[C@H]2O)n1 ; O=c1ccn([C@@H]2C3=CCC[C@@H](O)[C@@H]3[C@@H](O)[C@H]2O)c(=O)[nH]1 ; NC(=O)c1ncnn1[C@@H]1C2=CC[C@H](O)C[C@@H]2[C@@H](O)[C@H]1O ; NC(=O)c1ncn([C@@H]2C3=CC[C@H](O)C[C@@H]3[C@@H](O)[C@H]2O)n1 ; O=c1ccn([C@@H]2C3=CC[C@H](O)C[C@@H]3[C@@H](O)[C@H]2O)c(=O)[nH]1]

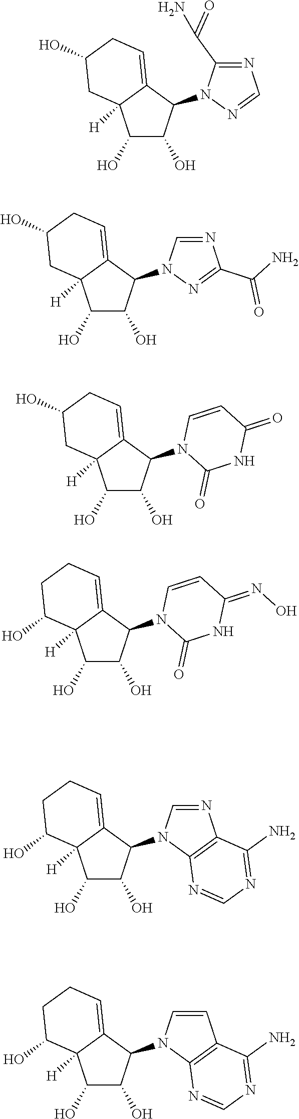 [SMILES: NC(=O)c1ncnn1[C@@H]1C2=CC[C@@H](O)C[C@@H]2[C@@H](O)[C@H]1O ; NC(=O)c1ncn([C@@H]2C3=CC[C@@H](O)C[C@@H]3[C@@H](O)[C@H]2O)n1 ; O=c1ccn([C@@H]2C3=CC[C@@H](O)C[C@@H]3[C@@H](O)[C@H]2O)c(=O)[nH]1 ; O=c1[nH]/c(=N\O)ccn1[C@@H]1C2=CCC[C@@H](O)[C@@H]2[C@@H](O)[C@H]1O ; Nc1ncnc2c1ncn2[C@@H]1C2=CCC[C@@H](O)[C@@H]2[C@@H](O)[C@H]1O ; Nc1ncnc2c1ccn2[C@@H]1C2=CCC[C@@H](O)[C@@H]2[C@@H](O)[C@H]1O]

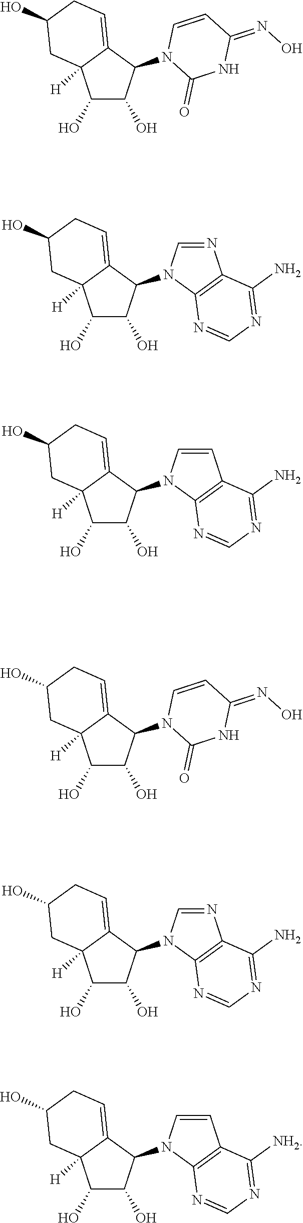 [SMILES: O=c1[nH]/c(=N\O)ccn1[C@@H]1C2=CC[C@H](O)C[C@@H]2[C@@H](O)[C@H]1O ; Nc1ncnc2c1ncn2[C@@H]1C2=CC[C@H](O)C[C@@H]2[C@@H](O)[C@H]1O ; Nc1ncnc2c1ccn2[C@@H]1C2=CC[C@H](O)C[C@@H]2[C@@H](O)[C@H]1O ; O=c1[nH]/c(=N\O)ccn1[C@@H]1C2=CC[C@@H](O)C[C@@H]2[C@@H](O)[C@H]1O ; Nc1ncnc2c1ncn2[C@@H]1C2=CC[C@@H](O)C[C@@H]2[C@@H](O)[C@H]1O ; Nc1ncnc2c1ccn2[C@@H]1C2=CC[C@@H](O)C[C@@H]2[C@@H](O)[C@H]1O]

In another aspect, the disclosure also provides a compound comprising one of the following formulas, or a pharmaceutically acceptable salt thereof:

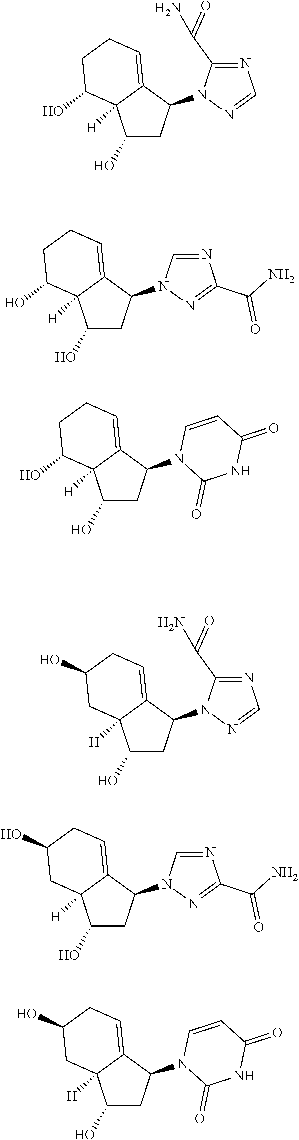 [SMILES: NC(=O)c1ncnn1[C@H]1C[C@H](O)[C@@H]2C1=CCC[C@H]2O ; NC(=O)c1ncn([C@H]2C[C@H](O)[C@@H]3C2=CCC[C@H]3O)n1 ; O=c1ccn([C@H]2C[C@H](O)[C@@H]3C2=CCC[C@H]3O)c(=O)[nH]1 ; NC(=O)c1ncnn1[C@H]1C[C@H](O)[C@H]2C[C@@H](O)CC=C21 ; NC(=O)c1ncn([C@H]2C[C@H](O)[C@H]3C[C@@H](O)CC=C32)n1 ; O=c1ccn([C@H]2C[C@H](O)[C@H]3C[C@@H](O)CC=C32)c(=O)[nH]1]

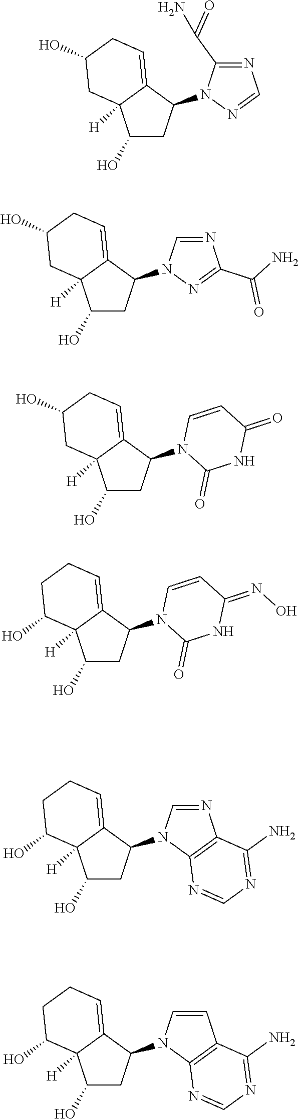 [SMILES: NC(=O)c1ncnn1[C@H]1C[C@H](O)[C@H]2C[C@H](O)CC=C21 ; NC(=O)c1ncn([C@H]2C[C@H](O)[C@H]3C[C@H](O)CC=C32)n1 ; O=c1ccn([C@H]2C[C@H](O)[C@H]3C[C@H](O)CC=C32)c(=O)[nH]1 ; O=c1[nH]/c(=N\O)ccn1[C@H]1C[C@H](O)[C@@H]2C1=CCC[C@H]2O ; Nc1ncnc2c1ncn2[C@H]1C[C@H](O)[C@@H]2C1=CCC[C@H]2O ; Nc1ncnc2c1ccn2[C@H]1C[C@H](O)[C@@H]2C1=CCC[C@H]2O]

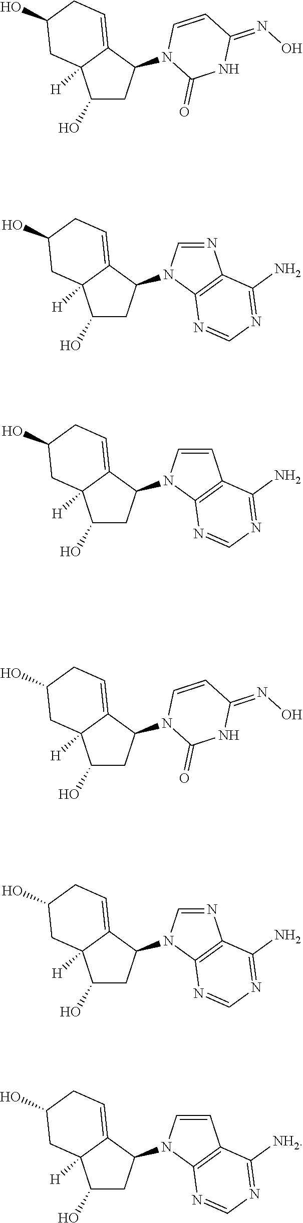 [SMILES: O=c1[nH]/c(=N\O)ccn1[C@H]1C[C@H](O)[C@H]2C[C@@H](O)CC=C21 ; Nc1ncnc2c1ncn2[C@H]1C[C@H](O)[C@H]2C[C@@H](O)CC=C21 ; Nc1ncnc2c1ccn2[C@H]1C[C@H](O)[C@H]2C[C@@H](O)CC=C21 ; O=c1[nH]/c(=N\O)ccn1[C@H]1C[C@H](O)[C@H]2C[C@H](O)CC=C21 ; Nc1ncnc2c1ncn2[C@H]1C[C@H](O)[C@H]2C[C@H](O)CC=C21 ; Nc1ncnc2c1ccn2[C@H]1C[C@H](O)[C@H]2C[C@H](O)CC=C21]

In some embodiments, a compound of the disclosure, or a pharmaceutically acceptable salt thereof, comprising the formula:

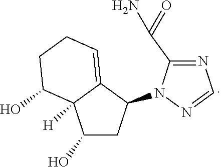 [SMILES: NC(=O)c1ncnn1[C@H]1C[C@H](O)[C@@H]2C1=CCC[C@H]2O]

In some embodiments, a compound of the disclosure, or a pharmaceutically acceptable salt thereof, comprising the formula:

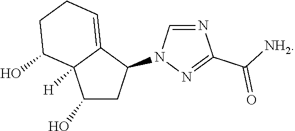 [SMILES: NC(=O)c1ncn([C@H]2C[C@H](O)[C@@H]3C2=CCC[C@H]3O)n1]

In some embodiments, a compound of the disclosure, or a pharmaceutically acceptable salt thereof, comprising the formula:

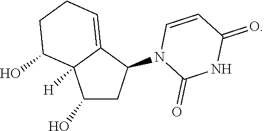 [SMILES: O=c1ccn([C@H]2C[C@H](O)[C@@H]3C2=CCC[C@H]3O)c(=O)[nH]1]

In another aspect, the disclosure provides a pharmaceutical composition comprising any of the compounds described herein, or a pharmaceutically acceptable salt thereof, and pharmaceutically acceptable excipient.

In another aspect, the disclosure provides a method for treating a viral infection, the method comprising administering to a subject in need thereof a therapeutically effective amount of a compound described herein, or a pharmaceutically acceptable salt thereof, or a therapeutically effective amount of a pharmaceutical composition described herein.

In some embodiments, the viral infection is a respiratory virus infection.

In some embodiments, the viral infection is a coronavirus infection, an adenovirus infection, an influenza virus infection, a parainfluenza virus infection, a rhinovirus infection, or a respiratory syncytial virus (RSV) infection. In particular embodiments, the coronavirus infection is a SARS-CoV-2 infection. In particular embodiments, the influenza virus infection is an influenza A virus (IAV) infection.

In some embodiments of the method, the compound is administered to the subject orally or via injection. In some embodiments, the method further comprises administering one or more agents selected from the group consisting of an anti-inflammatory agent, an analgesic agent, and an antiviral agent. The antiviral agent can be selected from the group consisting of an RNA-dependent RNA polymerase inhibitor, a 3C-like protease inhibitor, and a cytochrome P450 3A4 inhibitor.

### BRIEF DESCRIPTION OF THE DRAWINGS

FIGS. 1A and 1B show the effects of molnupiravir and carbobicyclic nucleoside 48 according to the present disclosure on infectivity (of SARS-CoV-2 S-pseudotyped lentivirus) (FIG. 1A) and cell viability (on A549-ACE2) (FIG. 1B) in an A549-ACE2 cell model. Experiments were performed in triplicate.

FIG. 2 shows that compound 48 inhibits the replication of different SARS-CoV-2 viral strains (including the original Wuhan strain and Delta and Omicron variants) in a A549-ACE2 cell model. Results for molnupiravir are also depicted. Experiments were done in triplicate.

FIGS. 3A-3D show antiviral activities of the compounds disclosed herein at different concentrations.

FIGS. 4A and 4B show cytotoxicity assays of the compounds disclosed herein at different concentrations.

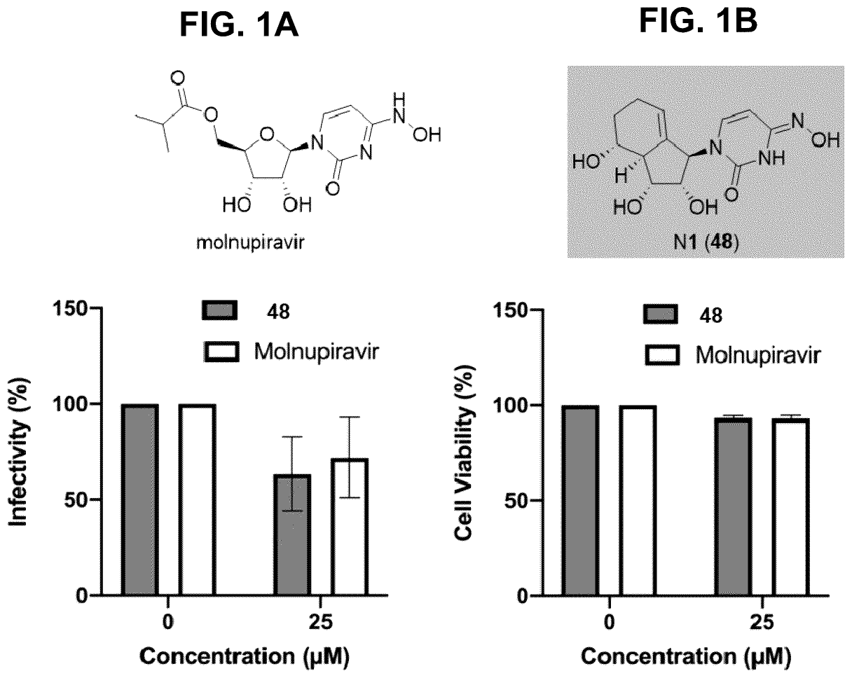

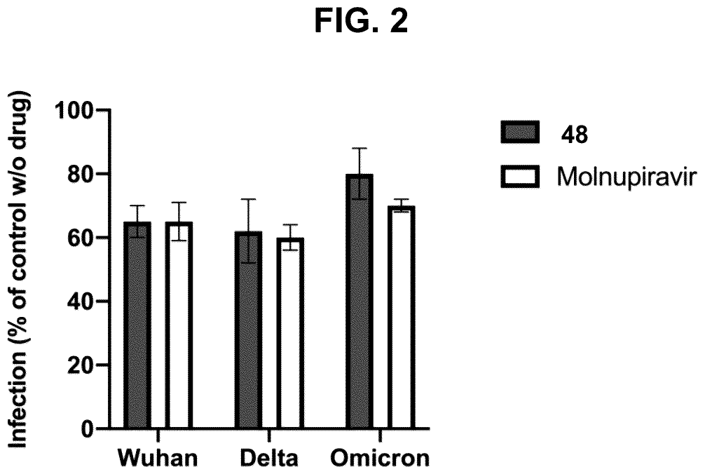

### DETAILED DESCRIPTION OF THE INVENTION

Provided herein are compounds that are useful for the treatment of viral infections (e.g., respiratory virus infections), including, but are not limited to, the treatment of COVID-19 caused by the SARS-CoV-2 and infections caused by influenza viruses (e.g., influenza A viruses (IAVs) and respiratory syncytial viruses (RSVs). An efficient and structure divergent route, featuring an intermolecular Diels-Alder reaction as a key step, has been developed to access several nucleoside analogues for the inhibition of viral replication. Advantageously, several compounds showing higher binding affinity than the current known inhibitor, molnupiravir, have now been discovered. Compounds according to the present disclosure contain a carbasugar instead of ribose as the core, rendering them metabolically more stable than molnupiravir and other existing ribose based inhibitors, such as anti-SARS-CoV-2 inhibitors.

### I. DEFINITIONS

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. “Substituted alkyl” groups can be substituted with one or more groups selected from halo, hydroxy, amino, alkylamino, amido, acyl, nitro, cyano, and alkoxy.

As used herein, the term “aryl,” by itself or as part of another substituent, refers to an aromatic ring system having any suitable number of ring atoms and any suitable number of rings. Aryl groups can include any suitable number of ring atoms, such as 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. “Substituted aryl” groups can be substituted with one or more groups selected from halo, hydroxy, amino, alkylamino, amido, acyl, nitro, cyano, and alkoxy.

As used herein, the term “arylalkyl” refers to an aryl group that is bonded to a compound via an alkylene group as described herein. Examples of arylalkyl groups include, but are not limited to, benzyl and phenethyl.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

As used herein, the terms “halo” and “halogen,” by themselves or as part of another substituent, refer to a fluorine, chlorine, bromine, or iodine atom.

As used herein, the term “cyano,” by itself or as part of another substituent, refers to a carbon atom triple-bonded to a nitrogen atom (i.e., the moiety —C≡N).

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino,” by itself or as part of another substituent, refers to a moiety —NR₃, wherein each R group is H or alkyl. An amino moiety can be ionized to form the corresponding ammonium cation.

As used herein, the term “hydroxy,” by itself or as part of another substituent, refers to the moiety —OH.

As used herein, the term “amido,” by itself or as part of another substituent, refers to a moiety —NRC(O)R or —C(O)NR₂, wherein each R group is H or alkyl.

As used herein, the term “acyl,” by itself or as part of another substituent, refers to a moiety —C(O)R, wherein R is alkyl.

As used herein, the term “nitro,” by itself or as part of another substituent, refers to the moiety —NO₂.

As used herein, the term “salt” refers to a compounds comprising at least one cation (e.g., an organic cation or an inorganic cation) and at least one anion (e.g., an organic anion or an inorganic anion). A salt may be an acid or base salt of a carbobicyclic nucleoside compound or another active agent. Acid salts of basic compounds include, but are not limited to, mineral acid salts (e.g., salts formed using hydrochloric acid, hydrobromic acid, phosphoric acid, and the like), organic acid salts (e.g., salts formed using acetic acid, propionic acid, glutamic acid, citric acid, and the like) salts, and quaternary ammonium salts (e.g., salts formed using methyl iodide, ethyl iodide, and the like). Acidic compounds may be contacted with bases to provide base salts such as alkali and alkaline earth metal salts (e.g., sodium, lithium, potassium, calcium, and magnesium salts), as well as ammonium salts (e.g., ammonium, trimethyl-ammonium, diethylammonium, and tris-(hydroxymethyl)-methyl-ammonium salts).

In some embodiments, the neutral forms of the active agents may be regenerated by contacting the salt with a base or acid and isolating the parent compound in the conventional manner if desired. In some embodiments, the parent form of the compound may differ from various salt forms in certain physical properties, such as solubility in polar solvents, but otherwise the salt forms may be equivalent to the parent form of the compound.

As used herein, the term “excipient” refers to a substance that aids the administration of an active agent to a subject. Pharmaceutical excipients useful in the present invention include, but are not limited to, binders, fillers, disintegrants, lubricants, glidants, coatings, sweeteners, flavors and colors.

By “pharmaceutically acceptable,” it is meant that the substance so designated (e.g., a salt or excipient) is not deleterious to the recipient thereof and is compatible with other substances with which it is formulated.

As used herein, the terms “treat,” “treatment,” and “treating” refer to any indicia of success in the treatment or amelioration of a viral infection or symptom (e.g., shortness of breath) thereof. Treatment may be assessed by objective or subjective parameters such as abatement; remission; diminishing of symptoms or making the symptom, injury, pathology or condition more tolerable to the patient; reduction in the rate of symptom progression; decreasing the frequency or duration of the symptom or condition. The treatment or amelioration of symptoms can be based on any objective or subjective parameter; including, e.g., the result of a physical examination.

As used herein, the term “administering” refers to oral, topical, parenteral, intravenous, intraperitoneal, intramuscular, intralesional, intranasal, subcutaneous, or intrathecal administration to a subject, as well administration as a suppository or the implantation of a slow-release device, e.g., a mini-osmotic pump, in the subject.

As used herein, the term “subject” refers to a person or other animal to whom a compound or composition as described herein is administered. In some embodiments, the subject is human.

As used herein, the terms “effective amount” and “therapeutically effective amount” refer to a dose of a compound such as a carbobicyclic nucleoside that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

The terms “about” and “around,” as used herein to modify a numerical value (e.g., degree of polymerization), indicate a close range surrounding that explicit value. If “X” were the value, “about X” or “around X” would indicate a value from 0.9X to 1.1X. “About X” thus includes, for example, a value from 0.95X to 1.05X, or from 0.98X to 1.02X, or from 0.99X to 1.01X. Any reference to “about X” or “around X” specifically indicates at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.07X, 1.08X, 1.09X, and 1.1 0X. Accordingly, “about X” and “around X” are intended to teach and provide written description support for a claim limitation of, e.g., “0.98X.”

### II. CARBOBICYCLIC NUCLEOSIDE ANALOGS

“Carbocyclic nucleoside” refers to a synthetic compound having a substituted cyclopentane ring where a naturally occurring nucleoside would have a furanose ring. A “carbobicyclic nucleoside” refers to compounds wherein the cyclopentane ring is fused to a second carbocyclic ring, such as cyclohexane ring in the case of octahydroindene-type carbobicyclic nucleosides. As described in more detail below, carbobicyclic nucleoside compounds have been developed and investigated for their potency and specificity against SARS-CoV-2 and RSV. The carbobicyclic nucleoside analogues can be prepared via a synthetic route employing an intermolecular Diels-Alder reaction.

The disclosure provides a compound of the following formula:

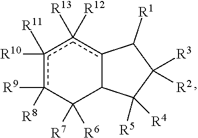

a pharmaceutically acceptable salt thereof, or an enantiomer thereof. In this formula, R¹ is nitrogen-containing monocyclic or bicyclic heterocyclyl; each of R², R³, R⁴, R⁵, R⁶, and R⁷ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, optionally substituted carboxylate, or OPₓ; each of R⁸, R⁹, R⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, OPₓ, or absent; Pₓ is monophosphate, diphosphate, triphosphate, C2-6 acyl, or a phosphate prodrug moiety; and each dashed line represents a single or double bond, provided that no more than one double bond is present.

Specifically, as the dashed line in the above formula represents an optional double bond, a compound of the disclosure can have any one of the following formulas:

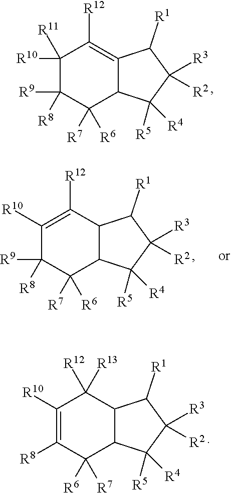

In some embodiments, a compound of the disclosure, or a pharmaceutically acceptable salt thereof, comprises the following formula:

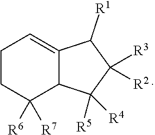

Specifically, a compound can have the following formula:

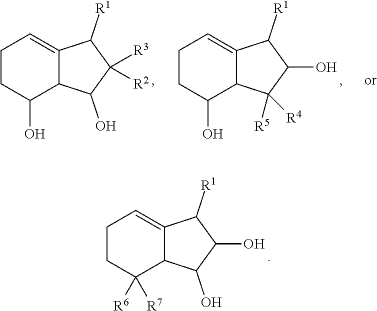

Further, a compound can have the following formula:

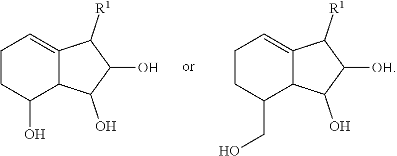

More specifically, a compound can have the following formula:

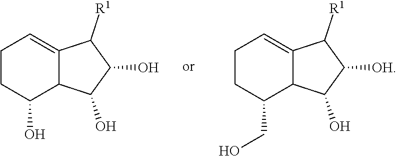

In some embodiments, a compound of the disclosure, or a pharmaceutically acceptable salt thereof, comprises the following formula:

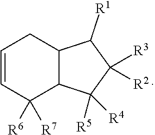

Specifically, a compound can have the following formula:

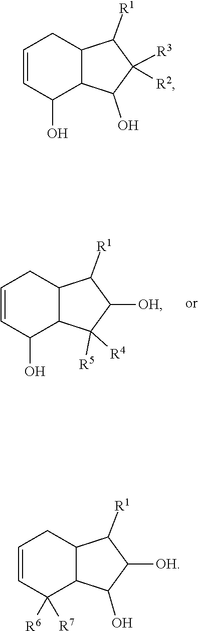

Further, a compound can have the following formula:

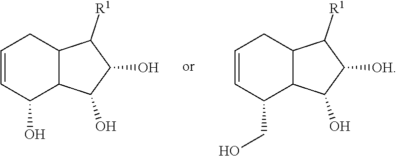

In some embodiments, in any of the formulas described above, each of R², R³, R⁴, and R⁵ is, independently, H, deuterium, hydroxyl, Cl, F, CN, Me, Et, (C₁₋₆)OH, ORₓ, (C₁₋₆)COORₓ, or (C₁₋₆)CON(Rₓ)₂; each of R⁶ and R⁷ is, independently, H, deuterium, hydroxyl, Cl, F, CN, Me, Et, (C₁₋₆)OH, ORₓ, (C₁₋₆)COORₓ, (C₁₋₆)CON(Rₓ)₂, optionally substituted carboxylate, or OPₓ; Pₓ is monophosphate, diphosphate, triphosphate, C₂₋₆ acyl, or a phosphate prodrug moiety; and each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy.

In some embodiments, a compound of the disclosure, or a pharmaceutically acceptable salt, can also have the following formula:

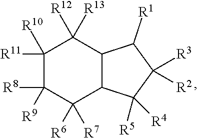

in which R¹ is optionally substituted nitrogen-containing monocyclic or bicyclic heterocyclyl; each of R², R³, R⁴, R⁵, R⁶, R⁷, R⁸, R⁹, R¹⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy, optionally substituted carboxylate, or OPₓ, wherein Pₓ is monophosphate, diphosphate, triphosphate, C2-6 acyl, or a phosphate prodrug moiety.

Particularly, in some embodiments, a compound can have the formula:

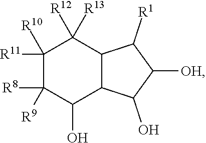

in which R¹ is optionally substituted nitrogen-containing monocyclic or bicyclic heterocyclyl; each of R¹⁰, R¹¹, R¹², and R¹³ is, independently, H, deuterium, hydroxyl, Cl, F, CN, Me, Et, (C₁₋₆)OH, ORₓ, (C₁₋₆)COORₓ, (C₁₋₆)CON(Rₓ)₂, epoxide, or cyclopropane; each of R⁸, R⁹ is, independently, H, deuterium, hydroxyl, Cl, F, CN, Me, Et, (C₁₋₆)OH, ORₓ, (C₁₋₆)COORₓ, (C₁₋₆)CON(Rₓ)₂, epoxide, cyclopropane, or OPₓ, Pₓ is monophosphate, diphosphate, triphosphate, C2-6 acyl, or a phosphate prodrug moiety, each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy.

In particular embodiments, a compound can have the following formula:

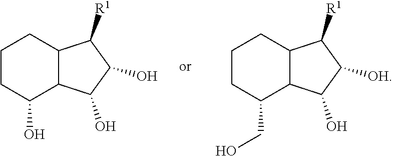

In any of the formulas described above, R¹ can be selected from the group consisting of adenine, guanine, cytosine, N⁴-hydroxycytosine, thymine, uracil, pyrrolo[2,1-f][1,2,4]triazin-4-amine, and 1,2,4-triazole-3-carboxamide. In some embodiments, R¹ can be selected from the group consisting of adenine, cytosine, and N⁴-hydroxycytosine.

In particular embodiments, in any of the formulas described herein, R¹ is

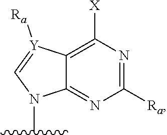

wherein each of X and Rₐ is, independently, H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy; and Y is N or C. In some embodiments, each of X and Rₐ is, independently, H, deuterium, hydroxyl, Cl, F, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy; and Y is N or C. In some embodiments, R¹ is:

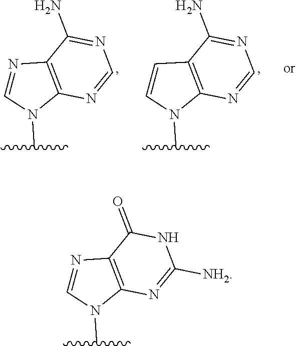

In particular embodiments, in any of the formulas described herein, R¹ is

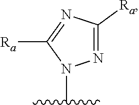

wherein each of Rₐ is, independently, H, deuterium, hydroxyl, Cl, F, CN, Me, Et, (C₁₋₆)OH, ORₓ, (C₁₋₆)COORₓ, CON(Rₓ)₂, (C₁₋₆)CON(Rₓ)₂, N(Rₓ)₂, or Rₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy. In some embodiments, R¹ is

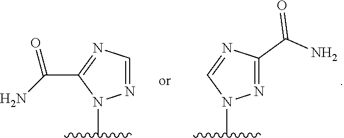 [SMILES: *n1ncnc1C(N)=O ; *n1cnc(C(N)=O)n1]

In particular embodiments, in any of the formulas described herein, R¹ is

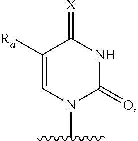

wherein Rₐ is H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ; and X is O, NRₓ, or NORₓ, wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy. In particular embodiments, R¹ is

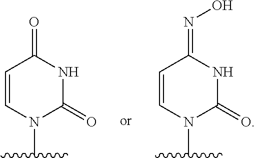 [SMILES: *n1ccc(=O)[nH]c1=O ; "or" ; *n1cc/c(=N/O)[nH]c1=O]

In any of the formulas described above, in some embodiments, Pₓ is a phosphate prodrug moiety. For example, a phosphate prodrug moiety can be selected from the group consisting of a phosphonate, carbonyloxymethyl phosphate ester, an S-acyl-2-thioethyl phosphate ester, an S-[(2-hydroxethyl)]sulfidyl]-thioethyl phosphate ester, a cyclosaligenyl phosphate diester, a cyclic 1-aryl-1,3-propanyl phosphate diester, an alkoxyalkyl phosphate ester, an aryloxy phosphoramidate, an aryloxy phosphonamidate, an amino acid phosphoramidate, an amino acid phosphonamidate, and a methylaryl haloalkyl phosphoramidate.

The disclosure provides a compound, or a pharmaceutically acceptable salt thereof, having any one of the following formulas:

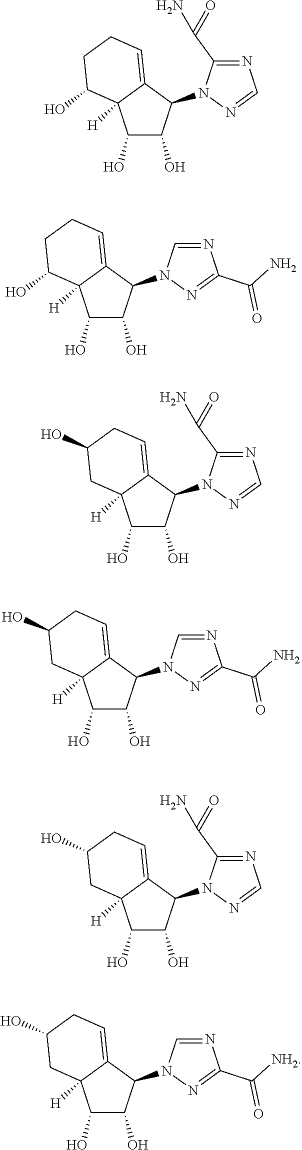 [SMILES: NC(=O)c1ncnn1[C@@H]1C2=CCC[C@@H](O)[C@@H]2[C@@H](O)[C@H]1O ; NC(=O)c1ncn([C@@H]2C3=CCC[C@@H](O)[C@@H]3[C@@H](O)[C@H]2O)n1 ; NC(=O)c1ncnn1[C@@H]1C2=CC[C@H](O)C[C@@H]2[C@@H](O)[C@H]1O ; NC(=O)c1ncn([C@@H]2C3=CC[C@H](O)C[C@@H]3[C@@H](O)[C@H]2O)n1 ; NC(=O)c1ncnn1[C@@H]1C2=CC[C@@H](O)C[C@@H]2[C@@H](O)[C@H]1O ; NC(=O)c1ncn([C@@H]2C3=CC[C@@H](O)C[C@@H]3[C@@H](O)[C@H]2O)n1]

In some embodiments, the compounds described herein can exist as a prodrug molecule, i.e., one or more hydroxyl groups in the compound can be protected as an optionally substituted carboxylate, such as

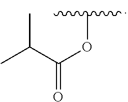 [SMILES: *OC(=O)C(C)C]

The disclosure provides a compound, or a pharmaceutically acceptable salt thereof, having any one of the following formulas:

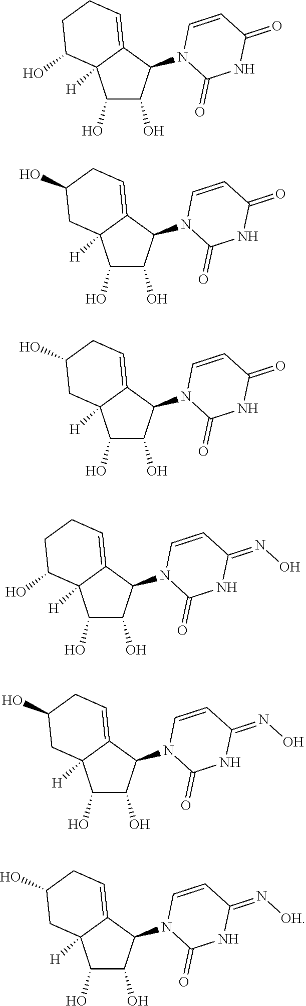

In some embodiments, the compounds described herein can exist as a prodrug molecule, i.e., one or more hydroxyl groups in the compound can be protected as an optionally substituted carboxylate, such as

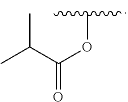 [SMILES: *OC(=O)C(C)C]

The disclosure provides a compound, or a pharmaceutically acceptable salt thereof, having any one of the following formulas:

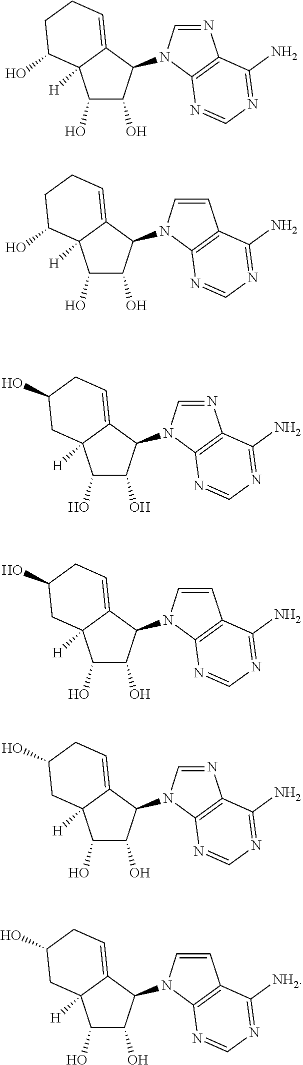

In some embodiments, the compounds described herein can exist as a prodrug molecule, i.e., one or more hydroxyl groups in the compound can be protected as an optionally substituted carboxylate, such as

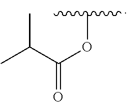 [SMILES: *OC(=O)C(C)C]

The disclosure provides a compound, or a pharmaceutically acceptable salt thereof, having any one of the following formulas:

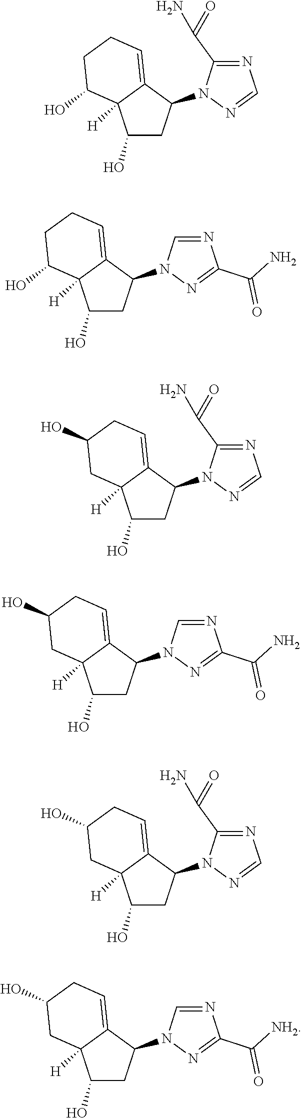 [SMILES: NC(=O)c1ncnn1[C@H]1C[C@H](O)[C@@H]2C1=CCC[C@H]2O ; NC(=O)c1ncn([C@H]2C[C@H](O)[C@@H]3C2=CCC[C@H]3O)n1 ; NC(=O)c1ncnn1[C@H]1C[C@H](O)[C@H]2C[C@@H](O)CC=C21 ; NC(=O)c1ncn([C@H]2C[C@H](O)[C@H]3C[C@@H](O)CC=C32)n1 ; NC(=O)c1ncnn1[C@H]1C[C@H](O)[C@H]2C[C@H](O)CC=C21 ; NC(=O)c1ncn([C@H]2C[C@H](O)[C@H]3C[C@H](O)CC=C32)n1]

In some embodiments, the compounds described herein can exist as a prodrug molecule, i.e., one or more hydroxyl groups in the compound can be protected as an optionally substituted carboxylate, such as

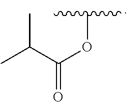 [SMILES: *OC(=O)C(C)C]

The disclosure provides a compound, or a pharmaceutically acceptable salt thereof, having any one of the following formulas:

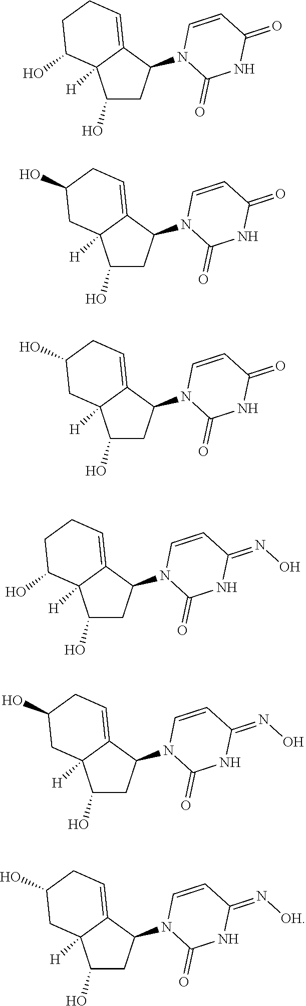

In some embodiments, the compounds described herein can exist as a prodrug molecule, i.e., one or more hydroxyl groups in the compound can be protected as an optionally substituted carboxylate, such as

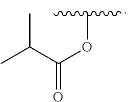 [SMILES: *OC(=O)C(C)C]

The disclosure provides a compound, or a pharmaceutically acceptable salt thereof, having any one of the following formulas:

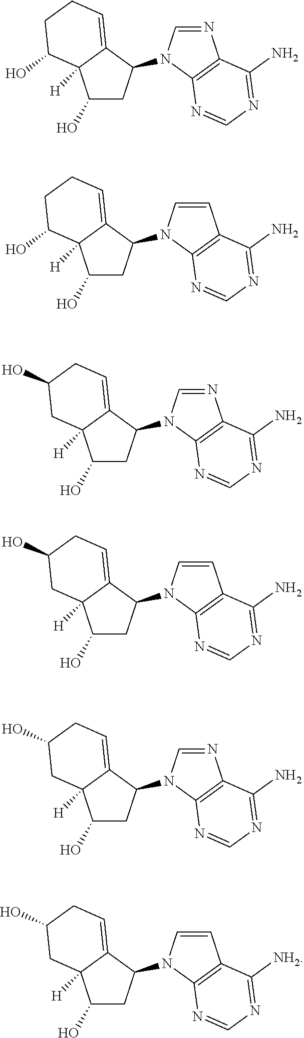

In some embodiments, the compounds described herein can exist as a prodrug molecule, i.e., one or more hydroxyl groups in the compound can be protected as an optionally substituted carboxylate, such as

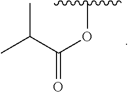 [SMILES: *OC(=O)C(C)C]

Provided herein are carbobicyclic nucleoside compounds according to the following formula:

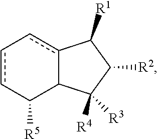

or a pharmaceutically acceptable salt thereof, wherein:
R¹ is nitrogen-containing monocyclic or bicyclic heterocyclyl;
R² is selected from the group consisting of —OH and —OR^{2a} wherein R^{2a} is C₂₋₆ acyl;
R³ is selected from the group consisting of —OH, —OR^{3a}, and halogen, wherein R^{3a} is C₂₋₆ acyl;
optionally R² and R³ are taken together to form a cyclic phosphate ester;
R⁴ is selected from the group consisting of hydrogen and halogen; and
(i) R⁵ is selected from the group consisting of —OH, —OR^{5a}, —CH₂OH, and —CH₂OR^{5a}, and
R^{5a} is selected from the group consisting of monophosphate, diphosphate, triphosphate, C₂₋₆ acyl, and a phosphate prodrug moiety,
or
(ii) R⁵ is hydrogen,
wherein each dashed line represents a single or double bond, provided that no more than one double bond is present.

In some embodiments of the formula above, R⁵ and R³ are —OH. In some embodiments, R⁵ is a phosphate prodrug moiety and R³ is —OH.

Phosphate prodrug moieties according to the present disclosure can be converted to active nucleoside triphosphates by the action of intracellular kinases, often in concert with other enzymes such as esterases, cytochrome P450s, and phospholipases.

Examples of phosphate prodrug moieties include, but are not limited to, phosphonates, carbonyloxymethyl phosphate esters, S-acyl-2-thioethyl phosphate esters, S-[(2-hydroxethyl)]sulfidyl]-thioethyl phosphate esters, cyclosaligenyl phosphate diesters, cyclic 1-aryl-1,3-propanyl phosphate diesters, alkoxyalkyl phosphate esters, aryloxy phosphoramidates, aryloxy phosphonamidates, amino acid phosphoramidate, amino acid phosphonamidates, and methylaryl haloalkyl phosphoramidates.

In some embodiments, the phosphate prodrug moiety is a carbonyloxymethyl phosphate or phosphonate as shown below:

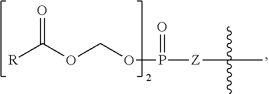

wherein each R is independently C₁₋₆ alkyl or C₁₋₆ alkoxy; —Z— is —O— or —CH₂—; and the wavy line represents the point of attachment between R² and the carbobicyclic nucleoside.

In some embodiments, the phosphate prodrug moiety is an S-acyl-2-thioethyl phosphate or phosphonate as shown below:

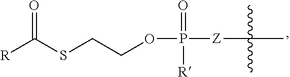

wherein R is C₁₋₆ alkyl or C₁₋₆ (hydroxy)alkyl; R′ is RC(O)SCH₂CH₂O—, C₁₋₆ alkoxy, C₁₋₆ aryloxy, or (C₇₋₂₀arylalkyl)amino; —Z— is —O— or —CH₂—; and the wavy line represents the point of attachment between R² and the carbobicyclic nucleoside.

In some embodiments, the phosphate prodrug moiety is an S-[(2-hydroxethyl)]sulfidyl]-thioethyl phosphate or phosphonate as shown below:

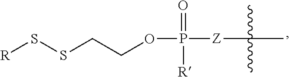

wherein R is C₁₋₆ alkyl; R′ is RSSCH₂CH₂O—, C₁₋₆ alkoxy, C₁₋₆ aryloxy, or (C₇₋₂₀arylalkyl)amino; —Z— is —O— or —CH₂—, and the wavy line represents the point of attachment between R² and the carbobicyclic nucleoside.

In some embodiments, the phosphate prodrug moiety is a cyclosaligenyl phosphate or phosphonate as shown below:

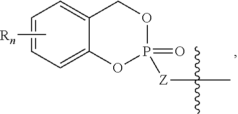

wherein subscript n is an integer ranging from 0 to 4; each R is independently hydrogen, halogen, or C₁₋₆ alkyl; —Z— is —O— or —CH₂—, and the wavy line represents the point of attachment between R² and the carbobicyclic nucleoside.

In some embodiments, the phosphate prodrug moiety is a cyclic 1-aryl-1,3-propanyl phosphate or phosphonate as shown below:

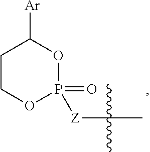

wherein Ar is C₆₋₁₄ aryl or 5-to-12-membered heteroaryl, each of which is optionally substituted with one or more independently selected halogen, or C₁₋₆ alkyl; —Z— is —O— or —CH₂—; and the wavy line represents the point of attachment between R² and the carbobicyclic nucleoside.

In some embodiments, the phosphate prodrug moiety is an alkoxyalkyl phosphate or phosphonate as shown below:

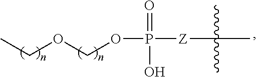

wherein m and n are independently integers ranging from 1 to 16; —Z— is —O— or —CH₂—; and the wavy line represents the point of attachment between R² and the carbobicyclic nucleoside.

In some embodiments, the phosphate prodrug moiety is an aryloxy amino acid phosphoramidate or phosphonamidate as shown below:

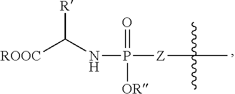

wherein R is C₁₋₆ alkyl; R′ is an amino acid side chain; R″ is C₆₋₁₄ aryl; —Z— is —O— or —CH₂—; and the wavy line represents the point of attachment between R² and the carbobicyclic nucleoside. R′ may represent, for example, the side chain of a naturally occurring amino acid (e.g., an alanine side chain, an arginine side chain, an asparagine side chain, an aspartic acid side chain, a cysteine side chain, a glutamine side chain, a glutamic acid side chain, a glycine side chain, a histidine side chain, an isoleucine side chain, a leucine side chain, a lysine side chain, a methionine side chain, a phenylalanine side chain, a proline side chain, a selenocysteine side chain, a serine side chain, a threonine side chain, a tryptophan side chain, a tyrosine side chain, or a valine side chain) or the side chain of a non-naturally occurring amino acid (e.g., an azidohomoalanine side chain, a propargylglycine side chain, a p-acetylphenylalanine side chain, or the like).

In some embodiments, the phosphate prodrug moiety is a methylaryl haloalkyl phosphoramidate or phosphonamidate as shown below:

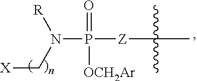

wherein R is hydrogen, C₁₋₆ alkyl, or C₁₋₆ (hydroxy)alkyl; X is halogen; n is an integer ranging from 1 to 16; Ar is C₆₋₁₄ aryl or 5-to-12-membered heteroaryl, each of which is optionally substituted with one or more groups independently selected from halogen, hydroxy, nitro, and C₁₋₆ alkyl; —Z— is —O— or —CH₂—, and the wavy line represents the point of attachment between R² and the carbobicyclic nucleoside.

In some embodiments, the carbobicyclic nucleoside has a structure selected from the group consisting of:

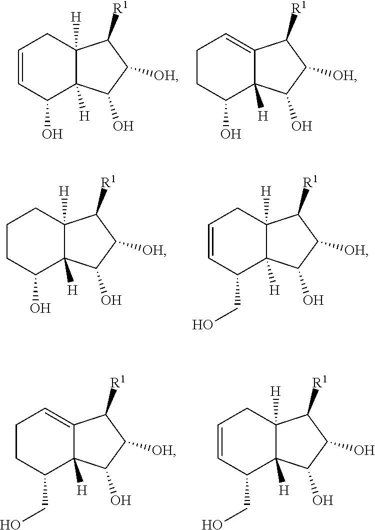

and pharmaceutically acceptable salts thereof.

In some embodiments of the formula above, R² is —OR^{2a}, and R³ and R⁵ are —OH. In some embodiments, R³ is —OR^{3a}, and R² and R⁵ are —OH. In some embodiments, R⁵ is —OR^{5a}, and R² and R³ are —OH. R^{2a}, R^{3a}, and R^{5a} may be, for example, acetyl, propionyl, n-butyryl, or iso-butyryl. In some such embodiments, R⁴ is H.

In some embodiments, R³ is hydrogen and R⁴ is halogen (e.g., fluorine, chlorine, or bromine). In some embodiments, R³ is halogen and R⁴ is hydrogen. In some embodiments, R³ and R⁴ are halogen. In some embodiments, R³ is fluorine and R⁴ is hydrogen. In some embodiments, R³ is hydrogen and R⁴ is fluorine.

Carbobicyclic nucleosides according to the present disclosure can contain a variety of naturally-occurring and synthetic nucleobases. R¹ may be, for example, a purine, a pyrimidine, a benzimidazole, or the like. In some embodiments, R¹ is adenine, guanine, 7-methylguanine, xanthine, or hypoxanthine. In some embodiments, R¹ is cytosine, 5-methylcytosine, 5-hydroxymethylcytosine, thymine, uracil, or 5,6-dihydrouracil. In some embodiments, R¹ is selected from the group consisting of adenine, guanine, cytosine, N⁴-hydroxycytosine, thymine, uracil, pyrrolo[2,1-f][1,2,4]triazin-4-amine, and 1,2,4-triazole-3-carboxamide. In some embodiments, R¹ is selected from the group consisting of adenine, cytosine, and N⁴-hydroxycytosine.

Also provided herein are processes for preparing the compounds described herein, as well as salts. By way of example, the compounds can be synthesized using the methods described below, together with synthetic methods known in the art of synthetic organic chemistry, or variations thereof. Preferred methods include, but are not limited to, the methods described in the working examples and the following Schemes 1 to 6. Starting materials and reagents used in preparing the compounds of the present disclosure are either available from commercial suppliers or are prepared by methods known to those skilled in the art following procedures set forth in references such as Fieser and Fieser's Reagents for Organic Synthesis, Vol. 1-28 (Wiley, 2016); March's Advanced Organic Chemistry, 7^{th} Ed. (Wiley, 2013); and Larock's Comprehensive Organic Transformations, 2^{nd} Ed. (Wiley, 1999). The starting materials and the intermediates of the reaction can be isolated and purified if desired using conventional techniques including, but not limited to, filtration, distillation, crystallization, chromatography, and the like. Such materials can be characterized using conventional means, including measuring physical constants and obtaining spectral data.

Those skilled in the art will recognize if a stereocenter exists in the compounds disclosed herein. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994).

As shown in Scheme 1, cyclopentenone (i) can be reduced to an enol which is substituted with heterocyclic R¹ group (e.g., under Mitsunobu-type conditions) to form cyclopentene acetal (ii). The acetal moiety can be removed and the resulting diol can be oxidized to form a ketol moiety in intermediate (iii). The cyclopentene moiety can then be employed as a dienophile for reaction with protected diene (iv) to provide Diels Alder product (v). Reduction of the ketol to form diol (vi) and subsequent deprotection provides the desired triol product.

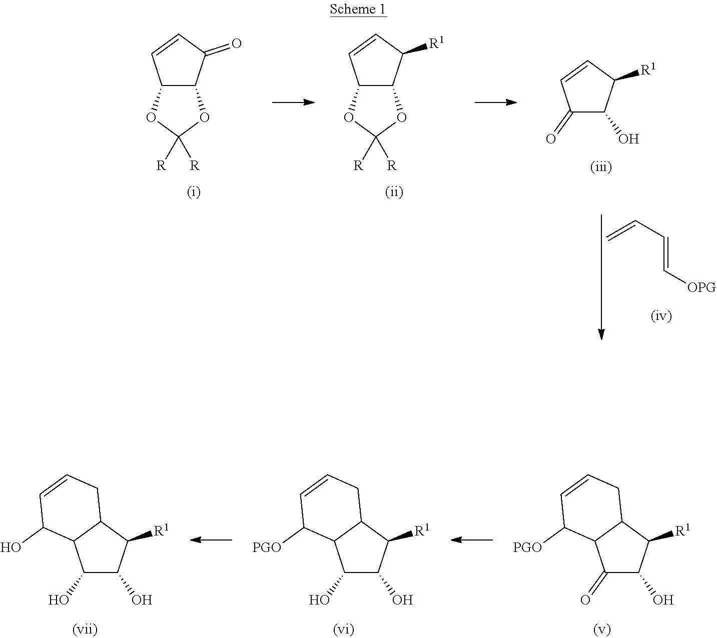

Alternatively, as shown in Scheme 2, cyclopentenone (i) can be converted to a diene (xiv) employed in Diels Alder reactions with a suitable dienophile. The cyclopentenone can be converted to halide (xi) and condensed with heterocyclic R¹ to from intermediate (xii). Reaction with stannane (xiii) in a Stille-type coupling provides diene (xiv) for reaction with boronate dienophile (xv) and formation of Diels Alder product (xvi). Hydrolysis of the boronate ester provides an alcohol R⁴ resp. R⁵, which can be converted to various phosphate prodrug moieties as described, for example, by Pradere et al. (Chemical Reviews, 2014, 114, 9154-9218), which is incorporated herein by reference. Deprotection of intermediate (xvii) provides the desired product (vii).

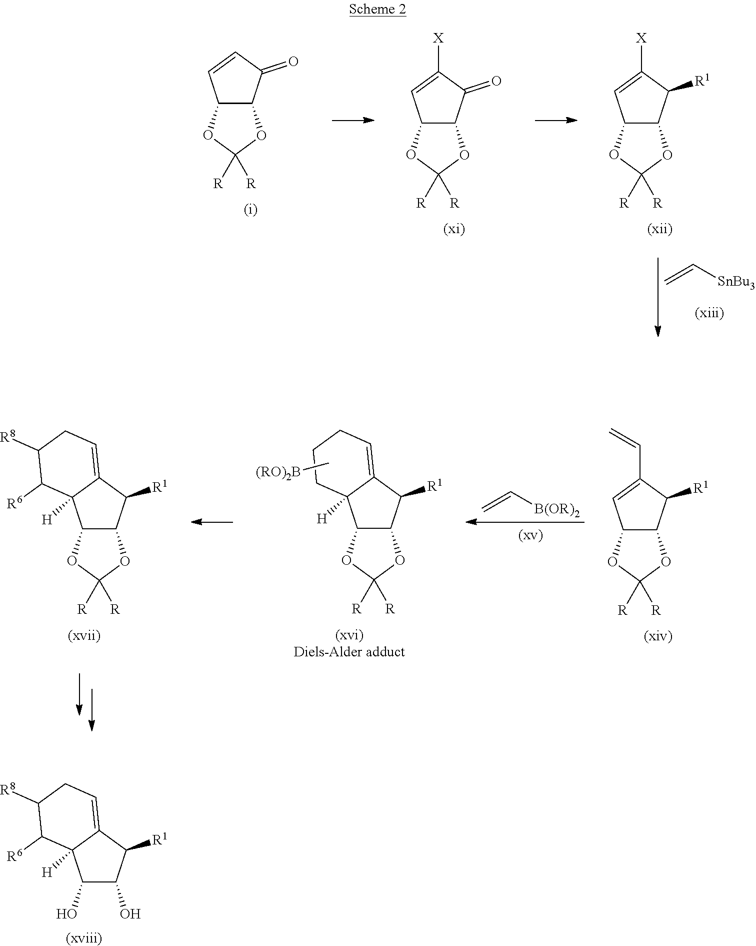

Alternatively, as shown in Scheme 3, enone (xxi) (Org. Synth. 1996, 73, 44) can be converted to xxv using the same conditions as described in Scheme 2. Halogenation to xxii is followed with coupling to xxii and Stille coupling to the diene (xxiii). Then Diels Alder reaction with xv delivers adduct xxiv which can be oxidized to xxv. Deprotection of intermediate (xxv) provides the desired product (xviii).

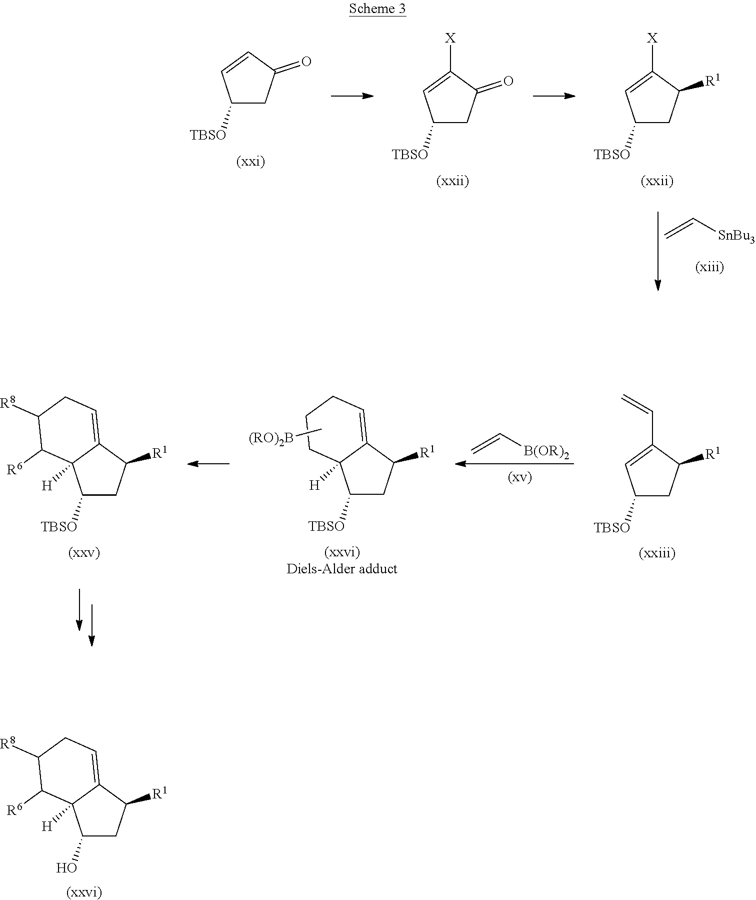

The unsaturated Diels Alder products shown in Scheme 1, Scheme 2 and Scheme 3 can be hydrogenated to provide compounds having a saturated carbobicyclic core.

Alternatively, as shown in Scheme 4, nucleoside analogues of the structure (vii) or (xviii) can be protected yielding two regioisomers (xxxi) and (xxxii) (e.g., PG=tert-butyl dimethyl silyl). The free alcohol can now be transformed into thioesters (xxxiv) and (xxxv) using xxxiii. Deoxygenation then take place under radical conditions to intermediates (xxxvi) and (xxxvii). Deprotection then provides the desired products (xxxviii) and (xxxiv). See, for example, Nandanan et al. (J. Med. Chem. 2000, 43, 5, 829-842) and Meier et al. (Synlett 1991, 1991, 4, 227-228).

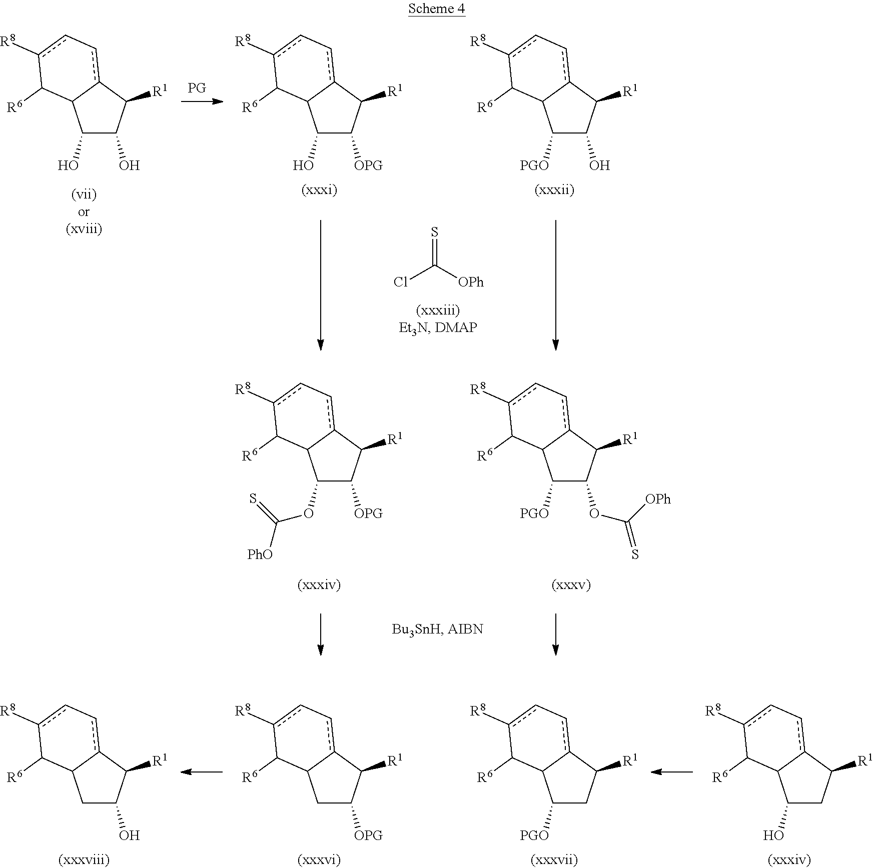

Halogenated compounds can be prepared as shown in Scheme 5. Described intermediates (xxxi) and (xxxii) can be directly treated with diethylaminosulfur trifluoride (DAST) yielding in monofluorides (xli) and (xlii). Deprotection then provides the desired products (xliii) and (xliv). Furthermore, difluorinated species can be achieved by oxidation to ketone (xlv) and (xlvi) followed by reaction with DAST to difluorides (xlvii) and (xlviii). Deprotection then provides the desired products (xlix) and (1). See, for example, Roy et al. (Bioorganic & Medicinal Chemistry, 2006, 14, 4980-4986) and Shi et al. (Bioorganic & Medicinal Chemistry 2005, 13, 1641-1652).

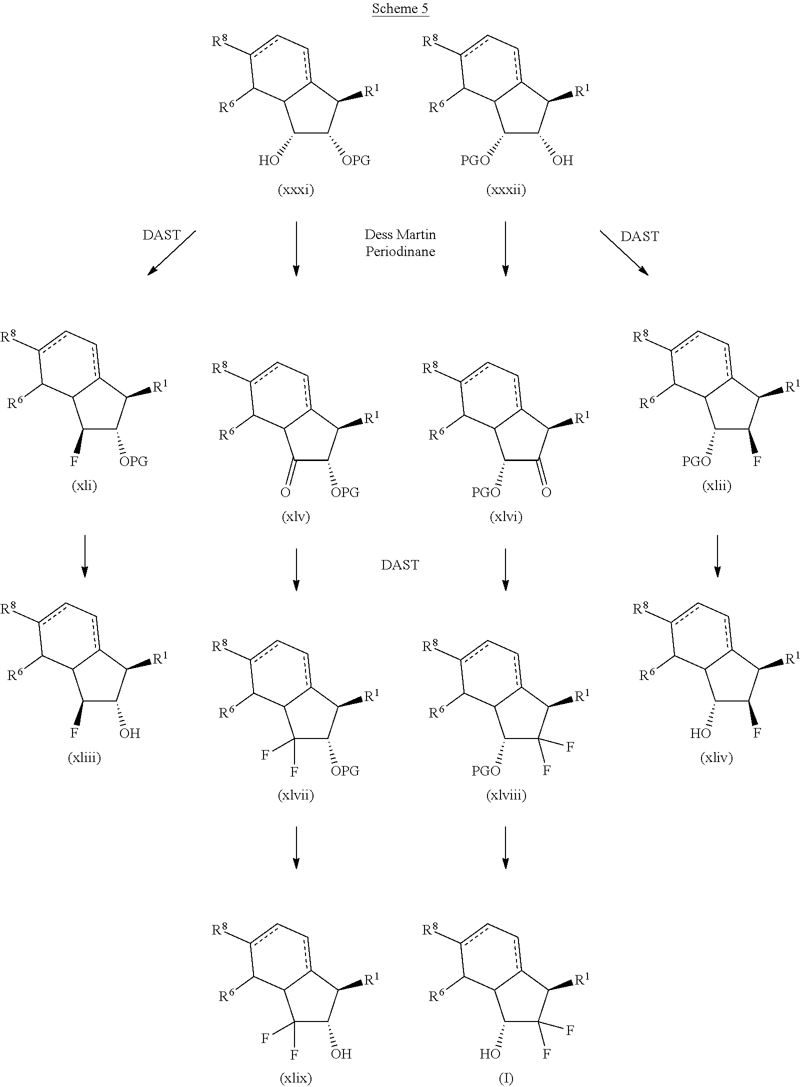

Alkylated compounds can be prepared as shown in Scheme 6. In Scheme 4 described (xxxi) and (xxxii) can be reacted with base and alkyl halide (e.g., Mel) providing (i) and (lii). Deprotection then provides the desired products (liii) and (liv). Additionally in Scheme 5 described (lv) and (lvi) can be reacted with Grignard reagent RMgX (e.g., MeMgBr) to synthesize (lv) and (lvi). Deprotection then provides the desired products (lvii) and (lviii).

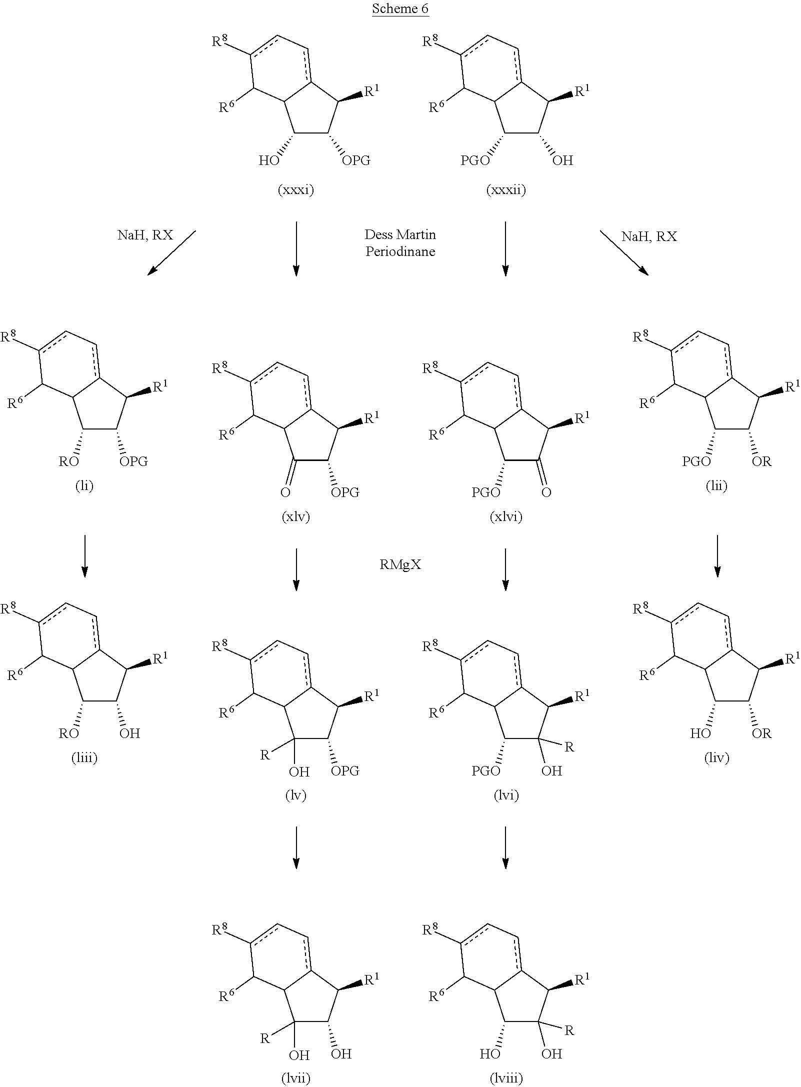

As used herein, the term “protecting group” refers to a chemical moiety that renders a functional group (e.g., a hydroxy group or an amino group) unreactive, but is also removable so as to restore the amino group. Examples of protecting groups include, but are not limited to, benzyloxy-carbonyl (Z or Cbz); 9-fluorenylmethyloxycarbonyl (Fmoc); tert-butyloxycarbonyl (Boc); allyloxycarbonyl (Alloc); p-toluene sulfonyl (Tos); 2,2,5,7,8-pentamethylchroman-6-sulfonyl (Pmc); 2,2,4,6,7-pentamethyl-2,3-dihydrobenzofuran-5-sulfonyl (Pbf); mesityl-2-sulfonyl (Mts); 4-methoxy-2,3,6-trimethylphenylsulfonyl (Mtr); acetamido; phthalimido; silyl groups such as trimethylsilyl, triethylsilyl, and (tert-butyl)dimethylsilyl; and the like. Other protecting groups are known to those of skill in the art including, for example, those described by Green and Wuts (Protective Groups in Organic Synthesis, 4^{th} Ed. 2007, Wiley-Interscience, New York).

### III. METHODS FOR THE TREATMENT OF COVID-19 AND OTHER VIRAL INFECTIONS

Also provided herein are methods for treating infections caused by viruses including coronaviruses and other respiratory viruses. The infection may be, for example, a coronavirus infection, an adenovirus infection, an influenza virus infection (e.g., influenza A virus (IAV) infection), a parainfluenza virus infection, a rhinovirus infection, or a respiratory syncytial virus (RSV) infection. The infection may be, but is not limited to, a SARS-CoV-2 infection, a SARS-CoV infection, a MERS-CoV infection, a HCoV-229E infection, a HCoV-OC43 infection, or a HCoV-NL63 infection. The methods include administering a therapeutically effective amount of a carbobicyclic nucleoside (e.g., a compound described herein), or a pharmaceutically acceptable salt thereof, to a subject in need thereof. In some embodiments, the subject has coronavirus disease 2019 (COVID-19).

In some embodiments, the subject is a human, an agricultural animal (e.g., livestock such as cows, sheep, pigs, or the like), or a companion animal (e.g., a pet such as a cat or dog). In some embodiments, the subject is a human over the age of 50 years old.

Carbobicyclic nucleoside compounds according to the present disclosure, as well as other active agents employed in combination therapy as described herein, can be administered to subject orally, intravenously, intramuscularly, intraperitoneally, subcutaneously, intrathecally, intraarterially, nasally, rectally, or via other routes if indicated. In some embodiments, the carbobicyclic nucleoside compound is administered orally or via injection. Active agents can be administered at any suitable dose in the methods provided herein. In general, a carbobicyclic nucleoside compound or other active agent is administered at a dose ranging from about 0.1 milligrams to about 1000 milligrams per kilogram of a subject's body weight (i.e., about 0.1-1000 mg/kg). The dose of the carbobicyclic nucleoside compound can be, for example, about 0.1-1000 mg/kg, or about 1-500 mg/kg, or about 25-250 mg/kg, or about 50-100 mg/kg. The dose of the carbobicyclic nucleoside compound can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. In some embodiments, the carbobicyclic nucleoside compound is administered in an amount ranging from about 0.1 mg/kg/day to about 100 mg/kg/day. In some embodiments, the carbobicyclic nucleoside compound is administered in an amount ranging from about 0.1 mg/kg/day to about 1.0 mg/kg/day. The dosages can be varied depending upon the requirements of the patient, the severity of the infection, the route of administration, and the particular formulation being administered. The dose administered to a patient should be sufficient to result in a beneficial therapeutic response in the patient. The size of the dose will also be determined by the existence, nature, and extent of any adverse side-effects that accompany the administration of the drug in a particular patient. Determination of the proper dosage for a particular situation is within the skill of the typical practitioner. The total dosage can be divided and administered in portions over a period of time suitable to treat to the COVID-19.

A carbobicyclic nucleoside compound or other active agent can be administered for periods of time which will also vary depending upon the severity of the COVID-19, and the overall condition of the subject to whom the active agent is administered. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, once per week, twice per week, or three times per week. Following treatment, a subject can be monitored for changes in his or her condition and for alleviation of the symptoms of COVID-19. The dosage of the active agent can either be increased in the event the subject does not respond significantly to a particular dosage level, or the dose can be decreased if an alleviation of symptoms is observed, or if the COVID-19 has been remedied, or if unacceptable side effects are seen with a particular dosage. Treating COVID-19 according to the methods of present disclosure can include alleviating one or more symptoms including, but not limited to, fever, cough, and shortness of breath. In some embodiments, treating COVID-19 can prevent severe, life-threatening illnesses such as pneumonia.

The methods and compositions described herein also can be administered prophylactically in subjects at risk for infection with SARS-CoV-2, to reduce the risk of developing COVID-19.

Administration of carbobicyclic nucleoside compounds according to the methods provided herein may result in the reduction of levels of SARS-CoV-2 RdRp activity in a subject. For example, the levels of RdRp activity may be reduced by from about 25% to about 95%, or from about 35% to about 95%, or from about 40% to about 85%, or from about 40% to about 80% as compared to the corresponding levels of RdRp activity prior to the first administration of the active agent (e.g., 24 hours prior to the first administration of the active agent). In some embodiments, the carbobicyclic nucleoside binds to SARS-CoV-2 non-structural protein 12 (nsp12) in the RdRp complex so as to reduce RdRp activity.

In some embodiments, the methods further include administering an analgesic agent (including anti-inflammatory analgesic agents), an antiviral agent, and an antitussive agent, or a combination thereof to the subject. Examples of non-steroidal anti-inflammatory agents (NSAIDs) include, but are not limited to, aceclofenac, 5-amino salicylic acid, aspirin, celecoxib, dexibuprofen, diclofenac, diflunisal, etodolac, fenoprofen, flufenamic acid, flurbiprofen, ibuprofen, indomethacin, ketoprofen, ketorolac, loxoprofen, mefenamic acid, nabumetone, naproxen, nimesulide, sulindac, and pharmaceutically acceptable salts thereof.

NSAIDs can be effective for relieving symptoms such as fever and pain. Additional analgesic agents such as paracetamol (acetaminophen) may also be administered in conjunction with the carbobicyclic nucleoside compound. Examples of further antiviral agents include, but are not limited to, protease inhibitors (e.g., nirmatrelvir, ritonavir, lopinavir, saquinavir, indinavir, or the like), nucleic acid polymerase inhibitors (e.g., acyclovir, foscarnet, ganciclovir, ribavirin or the like), neuraminidase inhibitors (e.g., zanamivir, oseltamivir, or the like), interferons, and ion channel blockers (e.g., amantadine, rimantadine, or the like). Examples of antitussive agents include, but are not limited to, codeine, hydrocodone, benzonatate, dextromethorphan, and chlophedianol. In some embodiments, the methods include administering a CYP3A4 inhibitor to the subject. Administration of the CYP3A4 inhibitor can prevent premature metabolism of the carbobicyclic nucleoside and increase plasma concentration levels following administration orally or via another route. Examples of CYP3A4 inhibitors include, but are not limited to, aprepitant, azamulin, boceprevir, chlorzoxazone, cilostazol, cimetidine, ciprofloxacin, clotrimazole, cobicistat, conivaptan, crizotinib, cyclosporine, diltiazem, dronedarone, erythromycin, fluconazole, fluvoxamine, fosaprepitant, grapefruit juice, imatinib, istradefylline, itraconazole, ivacaftor, ketoconazole, lomitapide, posaconazole, ranitidine, ranolazine, ritonavir, telaprevir, telithromycin, ticagrelor, tofisopam, troleandomycin, verapamil, and voriconazole. It will be appreciated that an active agent may be classified in more than one category; ritonavir, for example, can be classified as a protease inhibitor and a CYP3A4 inhibitor.

In some embodiments, the carbobicyclic nucleoside is administered as a pharmaceutical composition containing at least one pharmaceutically acceptable excipient and the carbobicyclic nucleoside or a pharmaceutically acceptable salt thereof. A carbobicyclic nucleoside compound may be administered to the subject before administration of one or more additional actives, after administration of one or more additional actives, or concurrently with administration of one or more additional actives. An carbobicyclic nucleoside compound may be administered in a composition separate from one or more additional actives, or in a composition containing one or more additional actives. Also provided herein are compositions containing: (i) one or more carbobicyclic nucleoside compounds; (ii) one or more pharmaceutically acceptable excipients; and optionally (iii) optionally one or more additional active agents, each of which is independently an anti-inflammatory agent, an analgesic agent, an antiviral agent, or an antitussive agent. The compositions may be formulated, e.g., for oral administration, intravenous administration, intramuscular administration, intraperitoneal administration, subcutaneous administration, intrathecal administration, intraarterial administration, nasal administration, or rectal administration.

The pharmaceutical compositions can be prepared by any of the methods well known in the art of pharmacy and drug delivery. In general, preparation of the compositions includes the step of bringing the active ingredients into association with a carrier containing one or more accessory ingredients. The pharmaceutical compositions are typically prepared by uniformly and intimately bringing the active ingredients into association with a liquid carrier or a finely divided solid carrier or both, and then, if necessary, shaping the product into the desired formulation. The compositions can be conveniently prepared and/or packaged in unit dosage form.

The pharmaceutical compositions may be in a form suitable for oral use. Suitable compositions for oral administration include, but are not limited to, tablets, troches, lozenges, aqueous or oily suspensions, dispersible powders or granules, emulsions, hard or soft capsules, syrups, elixirs, solutions, buccal patches, oral gels, chewing gums, chewable tablets, effervescent powders, and effervescent tablets. Such compositions can contain one or more agents selected from sweetening agents, flavoring agents, coloring agents, antioxidants, and preserving agents in order to provide pharmaceutically elegant and palatable preparations.

Tablets generally contain the active ingredients in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release. Compositions for oral administration can be formulated as hard gelatin capsules wherein the active ingredient is mixed with an inert solid diluent (such as calcium carbonate, calcium phosphate, or kaolin), or as soft gelatin capsules wherein the active ingredients are mixed with water or an oil medium (such as peanut oil, liquid paraffin, or olive oil).

The pharmaceutical compositions can also be in the form of an injectable aqueous or oleaginous solution or suspension. Sterile injectable preparations can be formulated using non-toxic parenterally-acceptable vehicles including water, Ringer's solution, and isotonic sodium chloride solution, and acceptable solvents such as 1,3-butane diol. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

Aqueous suspensions contain the active agents in admixture with excipients suitable for the manufacture of aqueous suspensions. Such excipients include, but are not limited to: suspending agents such as sodium carboxymethylcellulose, methylcellulose, oleagino-propylmethylcellulose, sodium alginate, polyvinyl-pyrrolidone, gum tragacanth and gum acacia; dispersing or wetting agents such as lecithin, polyoxyethylene stearate, and polyethylene sorbitan monooleate; and preservatives such as ethyl, n-propyl, and p-hydroxybenzoate. Oily suspensions can be formulated by suspending the active ingredients in a vegetable oil, for example, arachis oil, olive oil, sesame oil or coconut oil, or in a mineral oil such as liquid paraffin. The oily suspensions can contain a thickening agent, for example beeswax, hard paraffin, or cetyl alcohol. These compositions can be preserved by the addition of an anti-oxidant such as ascorbic acid. Dispersible powders and granules (suitable for preparation of an aqueous suspension by the addition of water) can contain the active ingredients in admixture with a dispersing agent, wetting agent, suspending agent, or combinations thereof. Additional excipients can also be present.

The pharmaceutical compositions of the invention can also be in the form of oil-in-water emulsions. The oily phase can be a vegetable oil, for example olive oil or arachis oil, or a mineral oil, for example liquid paraffin or mixtures of these. Suitable emulsifying agents can be naturally-occurring gums, such as gum acacia or gum tragacanth; naturally-occurring phospholipids, such as soy lecithin; esters or partial esters derived from fatty acids and hexitol anhydrides, such as sorbitan monooleate; and condensation products of said partial esters with ethylene oxide, such as polyoxyethylene sorbitan monooleate.

Transdermal delivery can be accomplished by means of iontophoretic patches and the like. The active ingredients can also be administered in the form of suppositories for rectal administration of the drug. These compositions can be prepared by mixing the active agents with a suitable non-irritating excipient which is solid at ordinary temperatures but liquid at the rectal temperature and will therefore melt in the rectum to release the drug. Such materials include cocoa butter and polyethylene glycols.

### IV. EXAMPLES

### Example 1. Preparation of (1R,2S,3R,3aR,7R,7aR)-3-(6-amino-9H-purin-9-yl)-2,3,3a,4,7,7a-hexahydro-1H-indene-1,2,7-triol (7)

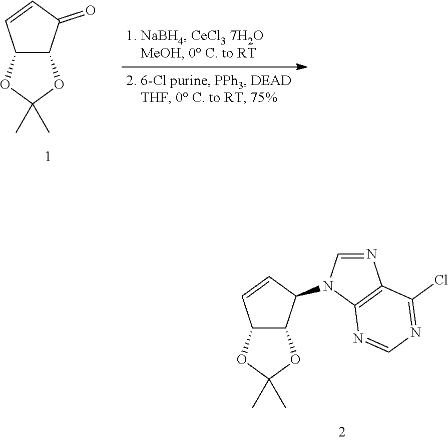 [SMILES: CC1(C)O[C@@H]2C=CC(=O)[C@@H]2O1 ; CC1(C)O[C@H]2[C@H](n3cnc4c(Cl)ncnc43)C=C[C@H]2O1]

To a stirred solution of enone 1 (50 mg, 0.32 mmol) and CeCl₃·7H₂O (179 mg, 0.48 mmol) in methanol (8 mL) at 0° C. was added NaBH₄ (15 mg, 0.39 mmol) in portions, and the resulted solution was kept at the same temperature for 30 min. Then the reaction was quenched with sat. aqueous NH₄Cl (30 mL) and extracted with DCM (15 mL×2). The combined organic phase was dried over Na₂SO₄ and concentrated to afford a crude allylic alcohol intermediate which was used directly for the next step.

To a stirred solution of above allylic alcohol (crude, 50 mg), 6-chloropurine (56 mg, 0.36 mmol) and PPh₃ (168 mg, 0.64 mmol) in anhydrous THF (8 mL) was added DEAD (0.1 mL, 0.64 mmol) at 0° C., and the resulting mixture was allowed to stir at room temperature for overnight. After TLC showed the completion of reaction, solvent was removed to afford a crude material which was purified by column chromatography (Hex/ethyl acetate 3:1) to provide compound 2 (70 mg, 75%, 2 steps). ¹H NMR (400 MHz, CDCl₃) ppm 8.78 (s, 1H), 7.99 (s, 1H), 6.40 (dt, J=5.6, 1.6 Hz, 1H), 5.98-5.96 (m, 1H), 5.70 (d, J=1.6 Hz, 1H), 5.31 (dd, J=5.6, 1.2 Hz, 1H), 4.71 (d, J=5.6 Hz, 1H), 1.50 (s, 3H), 1.36 (s, 3H).

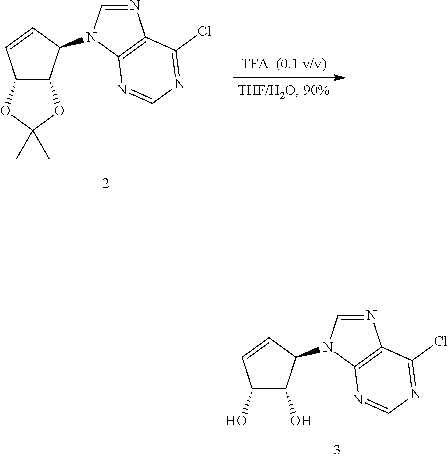 [SMILES: CC1(C)O[C@H]2[C@H](n3cnc4c(Cl)ncnc43)C=C[C@H]2O1 ; O[C@@H]1[C@H](O)C=C[C@H]1n1cnc2c(Cl)ncnc21]

To a stirred solution of compound 2 (430 mg, 1.47 mmol) in H₂O (10 mL) and THF (4 mL) was added TFA (0.09 mL), and the reaction was stirred at room temperature overnight. Then solvent was removed go give the crude product, which was purified by column chromatography on silica gel (DCM/MeOH 20:1) to afford diol 3 (330 mg, 90%) as a white solid. ¹H NMR (400 MHz, CDCl₃) ppm 8.74 (s, 1H), 8.12 (s, 1H), 6.35 (dt, J=6.0, 2.0 Hz, 1H), 6.13 (dd, J=6.0, 1.6 Hz, 1H), 5.56 (d, J=4.4, 1.6 Hz, 1H), 4.79 (d, J=6.0 Hz, 1H), 4.30 (t, J=5.2 Hz, 1H).

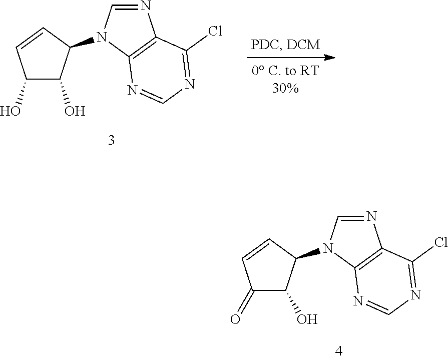 [SMILES: O[C@@H]1[C@H](O)C=C[C@H]1n1cnc2c(Cl)ncnc21 ; O=C1C=C[C@@H](n2cnc3c(Cl)ncnc32)[C@@H]1O]

To a stirred solution of compound 3 (30 mg, 0.12 mmol) in DCM (5 mL) and THF (1 mL) was added PDC (43 mg, 0.12 mmol) at 0° C., then the reaction was stirred at room temperature for 1.5 hr. Then the mixture was filtered, and the resulting crude material was purified by column chromatography on silica gel (DCM/MeOH 30:1) to provide enone 4 (9 mg, 30%, 36% BRSM) and recovered 4 (4 mg). ¹H NMR (400 MHz, CDCl₃) ppm 8.72 (s, 1H), 8.24 (s, 1H), 7.67 (d, J=4.8 Hz, 1H), 6.60 (dd, J=4.8 Hz, 1H), 5.58 (s, 1H), 4.68 (s, 1H).

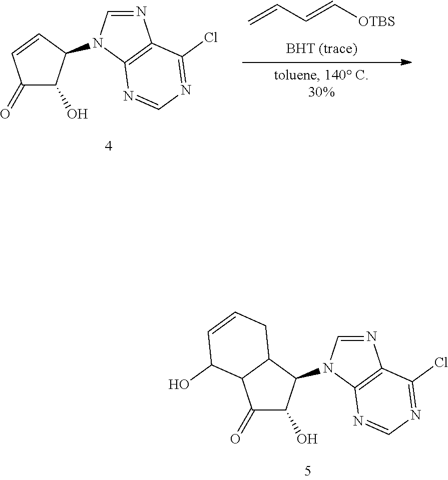

A mixture of enone 4 (5 mg, 0.02 mmol), diene and BHT (trace) in toluene in a sealed tube was heated to 140° C. under N₂ for 40 hrs, then solvent was removed to give the residue which was purified by column chromatography on silica gel (DCM/MeOH 40:1) to give enone 5 (3 mg, 30%) as a mixture of inseparable regio- and diastereomers. ¹H NMR (400M Hz, CDCl₃) ppm 8.83 (s, 0.5H), 8.75 (s, 1H), 8.62 (s, 0.6H), 8.32 (s, 1H), 6.15-6.11 (m, 1H), 6.08-6.04 (m, 1H), 5.14 (d, J=9.6 Hz, 1H), 4.82 (dd, J=8.8, 6.8 Hz, 1H), 4.66 (t, J=4.4 Hz, 1H), 3.30-3.25 (m, 2H), 2.98-2.94 (m, 1H), 2.76-2.74 (m, 1H), 2.00-1.94 (m, 3H), 0.91-0.86 (s, 9H), 0.07-0.03 (m, 6H). HRMS: Theoretical Mass [M+H]+ 435.1614, Found: 435.1615.

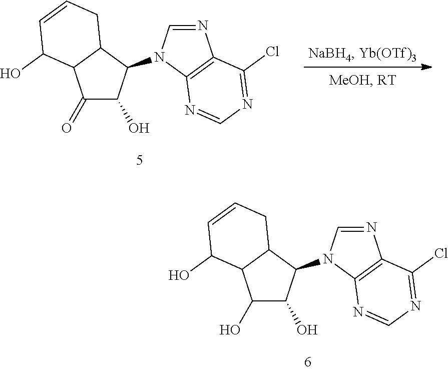 [SMILES: O=C1C2C(O)C=CCC2[C@@H](n2cnc3c(Cl)ncnc32)[C@@H]1O ; OC1C=CCC2C1C(O)[C@@H](O)[C@@H]2n1cnc2c(Cl)ncnc21]

To a stirred solution of ketone 5 (8 mg, 0.018 mmol) and Yb(OTf)₃ (12.4 mg, 0.02 mmol) in methanol was added NaBH₄ (˜1 mg, 0.025 mmol), and the resulting solution was kept at room temperature for 30 min. Then the reaction was quenched with sat. aqueous NH₄Cl (30 mL) and extracted with DCM (15 mL×2), the combined organic phase was dried over Na₂SO₄, concentrated to give the crude material which was purified by column chromatography on silica gel (DCM/MeOH 40:1) to give diol 6 (7 mg, 87%). ¹H NMR (400M Hz, CDCl₃) ppm 8.77 (s, 1H), 8.74 (s, 1H), 5.71-5.70 (m, 1H), 5.09 (dd, J=8.4, 2.4 Hz, 1H), 4.84-4.80 (m, 2H), 4.33-4.30 (m, 1H), 3.02-2.96 (m, 1H), 2.81-2.76 (m, 1H), 2.11-2.03 (m, 2H), 1.55-1.48 (m, 1H), 0.98 (s, 9H), 0.19 (s, 3H), 0.18 (s, 3H).

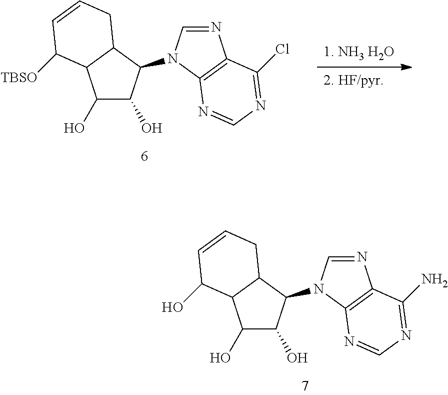 [SMILES: CC(C)(C)[Si](C)(C)OC1C=CCC2C1C(O)[C@@H](O)[C@@H]2n1cnc2c(Cl)ncnc21 ; "1. NH3 H2O" ; "2. HF/pyr." ; Nc1ncnc2c1ncn2[C@@H]1C2CC=CC(O)C2C(O)[C@H]1O]

To a sealed tube was added 6 (7 mg) and ammonia solution (0.5 mL) in THF (1 mL). The tube was heated to 105° C. under for 6 hr, and then the solvent was removed under vacuum to give a residue which was used directly for next step.

To a stirred solution of above crude (5 mg) in THF (2 mL) was added HF-Pyridine (70% in pyridine, 2 drops) at 0° C., then the reaction was allowed to warm to room temperature and stirred overnight. Solvent was removed to give the residue which was purified by column chromatography on silica gel (DCM/MeOH 40:1) to give triol 7 (1 mg). ¹H NMR (400M Hz, CDCl₃) ppm 8.36 (s, 1H), 8.23 (s, 1H), 5.86-5.83 (m, 1H), 5.75-5.72 (m, 1H), 4.74-4.70 (m, 2H), 4.37-4.35 (m, 1H), 2.95-2.91 (m, 1H), 1.91-1.86 (m, 1H), 1.59-1.53 (m, 1H). MS: 304.1 (M+H), 326.1 (M+Na)

### Example 2. Preparation of (1R,2S,3R,7R,7aR)-3-(6-amino-9H-purin-9-yl)-2,3,5,6,7,7a-hexahydro-1H-indene-1,2,7-triol (13)

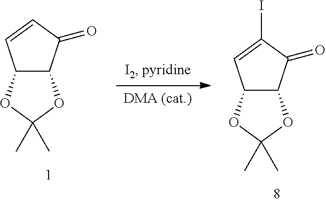

To a stirred solution of unsaturated ketone 1 (0.77 g, 5.00 mmol, 1.00 eq.) in dry DCM (25 mL) was added iodine (3.17 g, 12.5 mmol, 2.50 eq.). Then pyridine (0.50 mL, 6.25 mmol, 1.25 eq.) and DMAP (30.5 mg, 0.25 mmol, 5 mol %) were added subsequently at 0° C. The reaction was then stirred at room temperature. After 1.5 h the reaction was quenched by aq. Na₂S₂O₃ solution (15 g/50 mL H₂O) and extracted with DCM (20 mL×2). The combined organic phase was washed with brine (30 mL), dried over MgSO₄ and concentrated to give the crude which was purified by column chromatography on silica gel (Hex/EA 4:1). Iodide 8 was isolated as white crystalline solid (1.27 g, 0.45 mmol, 90%).

R_{f}=0.38 (Hex/EA 4:1); ¹H-NMR (400 MHz, CDCl₃) δ [ppm]=7.96 (d, J=2.6 Hz, 1H), 5.21 (dd, J=5.6, 2.6 Hz, 1H), 4.51 (d, J=5.6 Hz, 1H), 1.40 (s, 3H), 1.36 (s, 3H); ¹³C{¹H}-NMR (101 MHz, CDCl₃) δ [ppm]=197.7, 165.0, 115.9, 105.8, 79.8, 73.8, 27.4, 26.5.

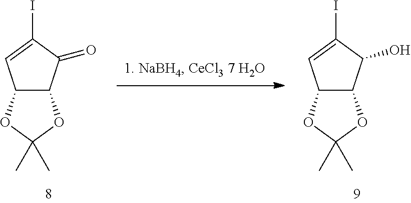

Unsaturated ketone 8 (1.28 g, 4.53 mmol, 1.00 eq.) was dissolved in MeOH (40 mL) and CeCl₃*7H₂O (1.94 g, 5.22 mmol, 1.15 eq.) was added. Then at 0° C. NaBH₄ (197 mg, 5.22 mmol, 1.15 eq.) was added. The reaction was stirred for 1 h, then quenched with sat. NH₄Cl solution (3 mL). The mixture was concentrated in vacuo and the crude was redissolved in an EtOAc/H₂O mixture (1:1, 40 mL). After the separation of the organic phase, the aqueous phase was extracted with EtOAc (3×15 mL). The combined organic phases were washed with brine (25 mL), dried over MgSO₄ and concentrated in vacuo. Alcohol 9 (1.18 g, 4.18 mmol, 92%) was isolated as a white solid in sufficient purity.

R_{f}=0.32 (Hex:EtOAc 4:1); ¹H-NMR (400 MHz, CDCl₃) δ [ppm]=6.31 (t, J=1.5 Hz, 1H), 4.92 (dd, J=1.8, 5.6 Hz, 1H), 4.69 (t, J=4.7 Hz, 1H), 4.42 (ddd, J=1.3, 5.6, 10.4 Hz, 1H), 2.81 (d, J=10.5 Hz, 1H), 1.43 (s, 3H), 1.40 (s, 3H).

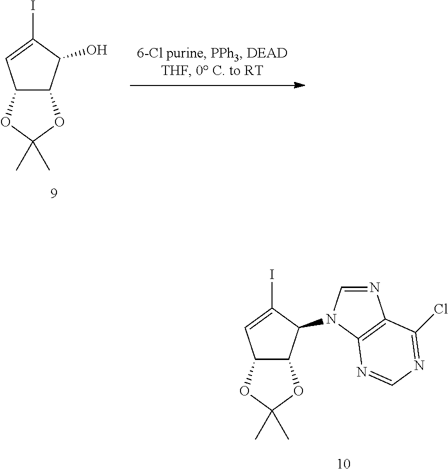 [SMILES: CC1(C)O[C@@H]2[C@@H](C=C(I)[C@@H]2O)O1 ; "6-Cl purine, PPh3, DEADTHF, 0° C. to RT" ; CC1(C)O[C@H]2[C@H](n3cnc4c(Cl)ncnc43)C(I)=C[C@H]2O1]

Intermediate 9 was converted to compound 10 using the procedure described for preparation of compound 2 in Example 1. ¹H NMR (400 MHz, CDCl₃) ppm δ 8.71 (s, 1H), 8.08 (s, 1H), 6.66 (s, 1H), 5.50 (d, J=1.6 Hz, 2H), 4.92 (d, J=6.1 Hz, 1H), 1.50 (s, 3H), 1.35 (s, 3H).

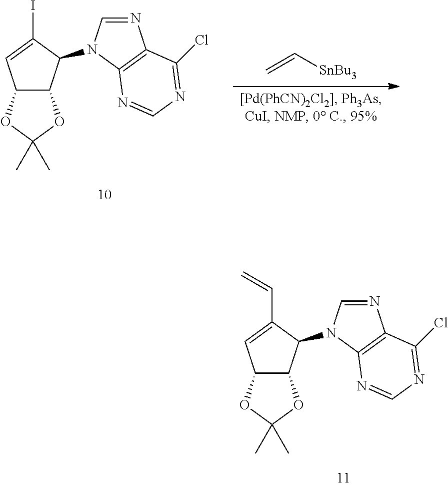

To a stirred solution of compound 10 (530 mg, 1.27 mmol), Pd(PhCN)₂Cl₂ (24 mg, 0.063 mmol, 0.05 eq.), Ph₃As (39 mg, 0.127 mmol, 0.1 eq.) and CuI (24 mg, 0.127 mmol, 0.1 eq) in NMP (10 mL) was added dropwise vinyl tributyl tin (0.45 mL, 1.52 mmol, 1.2 eq.) under N₂ at room temperature. After stirring at room temperature for 2 hrs, the reaction was quenched with water (80 mL) and extracted with ethyl acetate (15 mL×3). The combined organic phase were dried over Na₂SO₄ and concentrated to give a residue which was purified by column chromatography on silica gel (Hex/ethyl acetate 3:1) to provide diene 11 (383 mg, 95%). ¹H NMR (400 MHz, CDCl₃) ppm δ 8.79 (s, 1H), 7.88 (s, 1H), 6.48 (dd, J=17.8, 11.0 Hz, 1H), 6.27 (s, 1H), 5.89 (s, 1H), 5.49 (d, J=5.6 Hz, 1H), 5.18 (d, J=11.0 Hz, 1H), 4.98 (d, J=17.8 Hz, 1H), 4.65 (d, J=5.6 Hz, 1H), 1.46 (s, 3H), 1.33 (s, 4H).

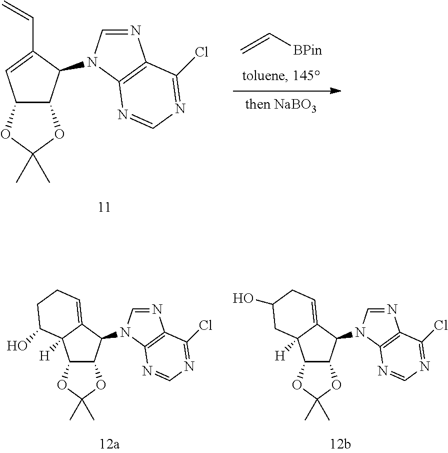

To a stirred solution of 11 (0.69 g, 2.17 mmol, 1.00 equiv.) BHT (47.8 mg, 0.22 mmol, 0.10 equiv.) was added. Then toluene (15 mL) and pinacol ester (1.10 mL, 6.50 mmol, 3.00 equiv.) was added. The reaction was heated in an oil bath (145° C.) for 3 days. Then the volatiles were removed in vacuo. The crude was dissolved in pH 7 buffer (10 mL), THF (10 mL) and NaBO₃·4H₂O (1.33 g, 8.67 mmol, 4.00 equiv.) was added. The reaction was stirred at room temperature for 2 hours and was then quenched with saturated sodium thiosulfate solution (10 mL) and stirred for 10 minutes. After phase separation, the aqueous layer was extracted with ethyl acetate (3×15 mL), and the combined organic phases were dried over MgSO₄ and filtered. Solvent was removed in vacuo, and the crude product was purified by silica gel column chromatography (hexane/ethyl acetate 1:1, DCM/ethyl acetate 1:2) to yield 12a and 12b (175 mg and 355 mg respectively, 1.46 mmol, 67% total yield). R_{f} 0.31, 0.21 (DCM/ethyl acetate 1:2); ¹H-NMR (600 MHz, CDCl₃) δ [ppm]=8.74 (s, 1H), 8.16 (s, 1H), 5.50 (s, 1H), 5.04 (t, J=5.86 Hz, 1H), 4.92 (s, 1H), 4.80 (t, J=6.17 Hz, 1H), 3.69 (t, J=10.23 Hz, 1H), 2.72 (s, 1H), 2.00-2.29 (m, 4H), 1.82 (s, 1H), 1.49-1.70 (m, 4H), 1.24-1.40 (m, 3H); ¹³C{¹H}-NMR (150 MHz, CDCl₃) δ [ppm]=152.2, 151.7, 151.5, 144.7, 136.6, 132.0, 122.0, 114.5, 83.7, 82.7, 71.7, 63.6, 52.7, 31.2, 27.5, 25.2, 25.0

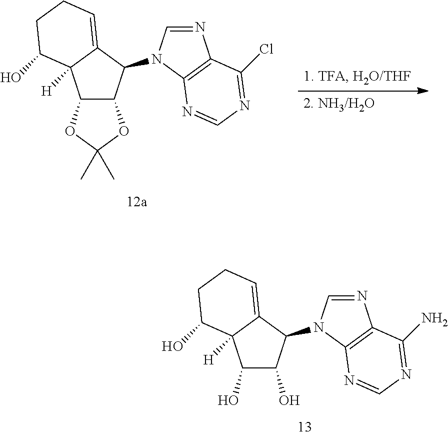 [SMILES: CC1(C)O[C@@H]2[C@H](O1)[C@@H]1C(=CCC[C@H]1O)[C@H]2n1cnc2c(Cl)ncnc21 ; "2. NH3/H2O" ; "1. TFA, H2O/THF" ; Nc1ncnc2c1ncn2[C@@H]1C2=CCC[C@@H](O)[C@@H]2[C@@H](O)[C@H]1O]

To a stirred solution of 12a (18 mg) in THF (2 mL) and H₂O (2 mL) was added TFA (0.02 mL), and the resulting mixture was stirred at room temperature overnight. Solvents were then removed to give a crude residue which was used without further purification. To the crude residue in a sealed tube was added THF (1 mL) and aqueous ammonia (0.8 mL), and the tube was heated to 120° C. under N₂ overnight. Then solvent was removed to afford the residue which was purified by column chromatography on silica gel (DCM/MeOH 7:1) to obtain compound 13 (14 mg, 94%). 15: ¹H NMR (400M Hz, MeOD) ppm 8.17 (s, 1H), 8.15 (s, 1H), 5.36 (m, 1H), 4.94 (m, 1H), 4.38 (t, J=6.8 Hz, 1H), 4.24 (t, J=4.9 Hz, 1H), 3.66 (m, 1H), 2.55 (m, 1H), 2.18-2.15 (m, 2H), 1.96-1.95 (m, 1H), 1.64-1.59 (m, 1H); ¹³C{¹H}-NMR (150 MHz, MeOD) δ [ppm]=155.9, 152.2, 152.0, 149.8, 140.4, 134.7, 120.0, 118.7, 74.7, 71.0, 70.3, 62.3, 52.5, 31.0, 24.5; MS (ESI+): m/z=303.2 (M+H).

### Example 3. Preparation of (1R,2S,3R,7R,7aR)-3-(6-amino-9H-purin-9-yl)octahydro-1H-indene-1,2,7-triol (14)

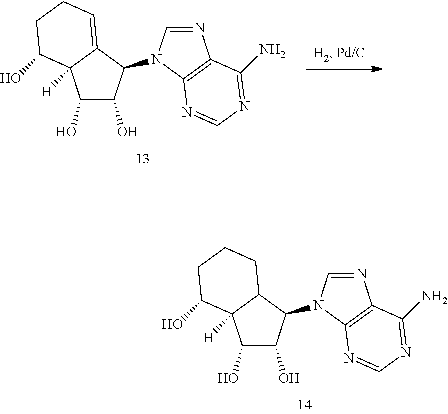 [SMILES: Nc1ncnc2c1ncn2[C@@H]1C2=CCC[C@@H](O)[C@@H]2[C@@H](O)[C@H]1O ; "H2, Pd/C" ; Nc1ncnc2c1ncn2[C@@H]1C2CCC[C@@H](O)[C@@H]2[C@@H](O)[C@H]1O]

To a solution of compound 13 (16 mg) in methanol (2 mL) was added Pd/C (wet, 15 mg), and the resulting solution was stirred under an H₂-filled balloon (1 atm) for 3 days. The mixture was filtered, and the filtrate was concentrated to give a residue which was purified by column chromatography on silica gel (DCM/MeOH 7:1) to afford product 14 (1 mg, 6%). With recovery of unreacted compound 13 (11 mg, 69%). 14: ¹H NMR (400M Hz, CDCl₃) ppm 8.30 (s, 1H), 8.19 (s, 1H), 4.77 (t, J=7.2 Hz, 1H), 4.35-4.32 (m, 1H), 4.14-4.10 (m, 2H), 2.69-2.64 (m, 1H), 2.23-2.16 (m, 1H), 1.99-1.92 (m, 2H), 1.67-1.60 (m, 2H), 0.89-0.87 (m, 2H). MS: 306.2 (M+H).

### Example 4. Preparation of (1R,2S,3R,6S,7aS)-3-(6-amino-9H-purin-9-yl)-2,3,5,6,7,7a-hexahydro-1H-indene-1,2,6-triol (16) and (1R,2S,3R,6S,7aS)-3-(6-methoxy-9H-purin-9-yl)-2,3,5,6,7,7a-hexahydro-1-indene-1,2,6-triol (17)

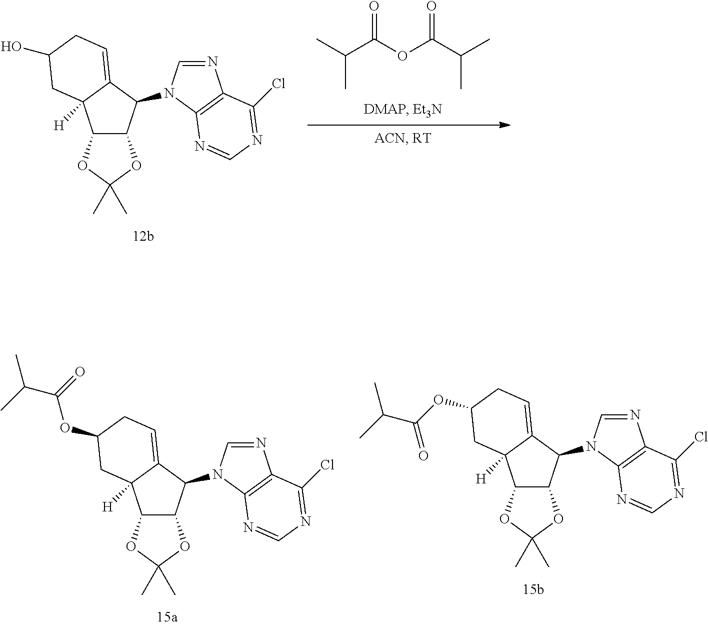

To a stirred solution of 12b (195 mg, 0.54 mmol, 1.00 equiv.) in acetonitrile (6 mL), triethylamine (118 μL, 0.85 mmol, 1.50 equiv.), isobutyric anhydride (141 μL, 0.85 mmol, 1.50 equiv.), and DMAP (6.92 mg, 0.06 mmol, 0.10 equiv.) was added at room temperature and stirred overnight. The reaction was quenched with NaHCO₃ (10 mL) and stirred for 10 minutes. After phase separation, the aqueous layer was extracted with ethyl acetate (4×5 mL), and the combined organic phases were dried over MgSO₄ and filtered. Solvent was removed in vacuo, and crude product was purified by silica gel column chromatography (hexane/ethyl acetate). Eluent ratio of 2:1 and 1:2 was used to yield 15a and 15b (132 mg and 36.8 mg respectively, 0.39 mmol, 72%). 15a: R_{f} 0.44 (hexane/ethyl acetate 2:1); ¹H-NMR (400 MHz, CDCl₃) δ [ppm]=8.74 (s, 1H), 8.14 (s, 1H), 5.47 (s, 1H), 4.87-5.12 (m, 3H), 4.57 (t, J=6.12 Hz, 1H), 2.88 (brs, 1H), 2.32-2.60 (m, 3H), 1.85-2.03 (m, 1H), 1.53-1.63 (m, 6H), 1.17-1.30 (m, 1H), 1.16 (d, J=1.75 Hz, 3H), 1.14 (d, J=1.84 Hz, 3H). 15b: R_{f} 0.24 (hexane/ethyl acetate 2:1); ¹H-NMR (600 MHz, CDCl₃) δ [ppm]=8.73 (s, 1H), 8.16 (s, 1H), 5.53 (s, 1H), 5.25 (s, 1H), 5.06 (t, J=5.78 Hz, 1H), 4.89 (s, 1H), 4.58 (t, J=6.23 Hz, 1H), 2.83 (s, 1H), 2.48-2.60 (m, 2H), 2.24-2.31 (m, 1H), 2.03-2.12 (m, 1H), 1.60 (s, 3H), 1.55 (t, J=12.61 Hz, 1H), 1.33 (s, 3H), 1.15 (dd, J 11.19 Hz, 6H); ¹³C{¹H}-NMR (150 MHz, CDCl₃) δ [ppm]=178.0, 156.0, 151.2, 150.6, 142.7, 137.9, 120.4, 119.8, 76.8, 76.5, 68.7, 64.3, 35.3, 31.2, 31.2, 19.2, 19.2

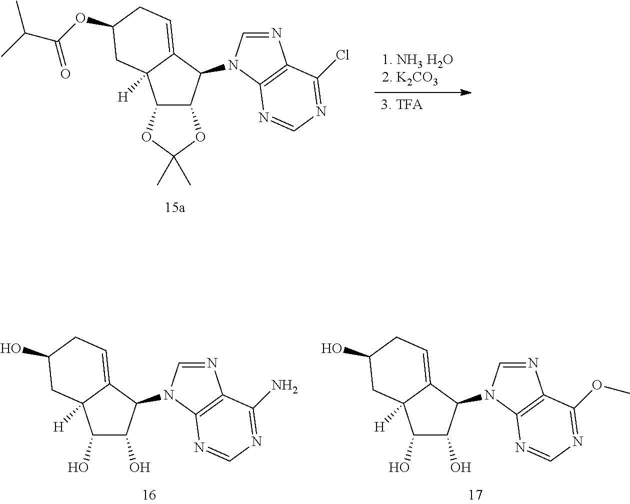

To a stirred solution of 15a (50.0 mg, 0.12 mmol, 1.00 equiv.) in a glass vial, acetonitrile (1 mL) and aqueous NH₃ (25% in water, 1 mL) was added and stirred for 1 day at 80° C. Residual water was removed in vacuo using acetonitrile (3×2 mL). Methanol (3 mL) and K₂CO₃ (24.2 mg, 0.18 mmol, 1.50 equiv.) was added at room temperature and stirred for 1 day. The reaction was quenched with brine (3 mL) and water (3 mL). After phase separation, the aqueous layer was extracted with ethyl acetate (3×5 mL), and the combined organic phases were dried over MgSO₄ and filtered. Methanol (0.5 mL), TFA (20% in water, 0.5 mL) and water (0.5 mL) was added and stirred for 1 day at room temperature. Solvent was removed in vacuo using acetonitrile (3×2 mL), and crude product was purified by silica gel column chromatography (methanol/ethyl acetate, 10%) to yield adenosine analogue 16 (22.0 mg, 0.07 mmol, 63%), with 17 as a side product (8.50 mg, 0.03 mmol). 16: R_{f} 0.07 (methanol/ethyl acetate, 5%); ¹H-NHR (600 MHz, MeOD-d4) δ [ppm]=8.19 (s, 1H), 8.14 (s, 1H), 5.31 (s, 1H), 5.07 (s, 1H), 4.31 (t, J=5.2 Hz, 1H), 4.06 (dd, J=5.9, 7.9 Hz, 1H), 3.95 (m, 1H), 2.75 (brs, 1H), 2.39 (m, 2H), 1.92 (m, 1H), 1.44 (q, J=11.9 Hz, 1H); ¹³C{¹H}-NMR (150 MHz, MeOD-d4) δ [ppm]=155.9, 152.2, 149.5, 140.5, 137.1, 120.0, 118.7, 75.9, 74.9, 66.8, 62.4, 44.0, 35.8, 33.9 17: R_{f} 0.13 (methanol/ethyl acetate, 5%); ¹H-NHR (600 MHz, MeOD-d4) δ [ppm]=8.52 (s, 1H), 8.33 (s, 1H), 5.39 (s, 1H), 5.03 (m, 1H), 4.35 (m, 1H), 4.20 (s, 3H), 4.11-4.15 (m, 1H), 3.91-3.98 (m, 1H), 2.77 (brs, 1H), 2.34-2.42 (m, 2H), 1.84-1.93 (m, 1H), 1.44 (q, J=17.4 Hz, 1H)

### Example 5. Preparation of (1R,2S,3R,6R,7aS)-3-(6-amino-9H-purin-9-yl)-2,3,5,6,7,7a-hexahydro-1H-indene-1,2,6-triol (18)

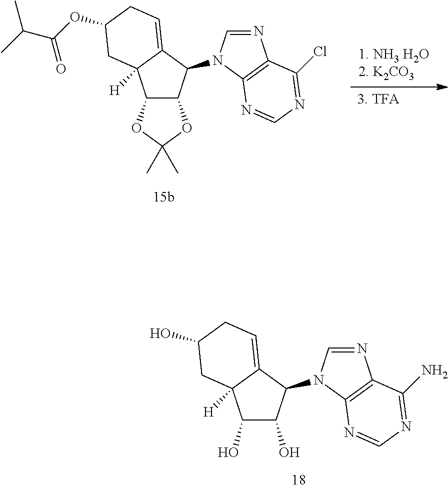 [SMILES: CC(C)C(=O)O[C@@H]1CC=C2[C@@H](n3cnc4c(Cl)ncnc43)[C@@H]3OC(C)(C)O[C@@H]3[C@H]2C1 ; "3. TFA" ; "1. NH3 H2O2. K2CO3" ; Nc1ncnc2c1ncn2[C@@H]1C2=CC[C@@H](O)C[C@@H]2[C@@H](O)[C@H]1O]

To a stirred solution of 15b (31.5 mg, 0.07 mmol, 1.00 equiv.) in a glass vial, acetonitrile (1 mL) and aqueous NH₃ (25% in water, 1 mL) was added and stirred for 1 day at 85° C. Residual water was removed in vacuo using acetonitrile (3×2 mL). Methanol (1 mL) and K₂CO₃ (15.8 mg, 0.11 mmol, 1.50 equiv.) was added at room temperature and stirred for 1 day. The reaction was quenched and diluted with brine (3 mL) and water (3 mL). After phase separation, the aqueous layer was extracted with ethyl acetate (3×5 mL), and the combined organic phases were dried over MgSO₄ and filtered. Methanol (0.5 mL), TFA (20% in water, 0.5 mL) and water (0.5 mL) was added and stirred for 1 day at room temperature. Solvent was removed in vacuo using acetonitrile (3×2 mL), and crude product was purified by silica gel column chromatography (methanol/ethyl acetate, 10%) to yield adenosine analogue 18 (7.90 mg, 0.03 mmol, 36%). R_{f} 0.05 (methanol/ethyl acetate, 10%); ¹H-NMR (600 MHz, MeOD-d4) δ [ppm]=8.19 (s, 1H), 8.14 (s, 1H), 5.36 (s, 1H), 5.04 (t, J=2.87 Hz, 1H), 4.32 (t, J=5.27 Hz, 1H), 4.21 (brs, 1H), 4.07 (m, 1H), 2.86 (brs, 1H), 2.27-2.34 (m, 2H), 2.08 (d, J=19.4 Hz, 1H), 1.50 (t, J=12.3 Hz, 1H).

### Example 6. Preparation of 1-((1R,2S,3R,3aR,4R)-2,3,4-trihydroxy-2,3,3a,4,5,6-hexahydro-1H-inden-1-yl)-1H-1,2,4-triazole-3-carboxamide (23)

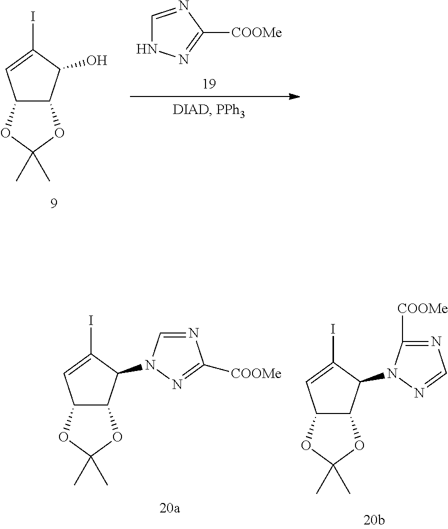

The alcohol 9 (1.00 g, 3.55 mmol, 1.00 eq.) was dissolved in DCM (50 mL). After the addition of the triazole 19 (0.52 g, 4.08 mmol, 1.15 eq.) and 4A MS the mixture was stirred for 2 h. Then at 0° C. first PPh₃ (1.12 g, 4.25 mmol, 1.20 eq.) and DIAD (0.84 mL, 4.25 mmol, 1.20 eq.) was added. The reaction was stirred for 18 h. Then the reaction was filtered, and the solvent was removed in vacuo. Purification by column chromatography (SiO₂, Hex:EtOAc 3:1 to 1:1) gave first 20b as a yellow oil (0.59 g, 1.51 mmol, 42%), later 20a as a white foam (0.49 g, 1.25 mmol, 36%). 20a: R_{f}=0.13 (Hex:EtOAc 3:1); ¹H-NMR (400 MHz, CDCl₃) δ [ppm]=8.26 (s, 1H), 6.60 (s, 1H), 5.36 (m, 2H), 4.89 (d, J=6.8 Hz, 1H), 3.99 (s, 3H), 1.47 (s, 3H), 1.34 (s, 3H); ¹³C{¹H}-NMR (101 MHz, CDCl₃) δ [ppm]=160.0, 156.1, 146.3, 145.3, 113.1, 96.1, 85.0, 82.5, 77.7, 53.0, 27.2, 25.9; HRMS (ESI, 3.5 kV) calc for [M+Na]⁺ C₁₂H₁₄IN₃O₄Na⁺ 413.99212, found 413.99171. 20b: R_{f}=0.24 (Hex:EtOAc 3:1); ¹H-NMR (400 MHz, CDCl₃) δ [ppm]=8.03 (s, 1H), 6.58 (s, 1H), 6.54 (s, 1H), 5.35 (dt, J=1.8, 5.7 Hz, 1H), 4.84 (d, J=5.8 Hz, 1H), 4.06 (s, 3H), 1.50 (s, 3H), 1.37 (s, 3H); ³C{¹H}-NMR (101 MHz, CDCl₃) δ [ppm]=158.3, 151.9, 145.4, 145.2, 112.9, 96.9, 85.3, 83.0, 76.8, 53.5, 27.4, 26.1; HRMS (ESI, 3.5 kV) calc for [M+Na]⁺ C₁₂H₁₄IN₃O₄Na⁺ 413.99212, found 413.99183.

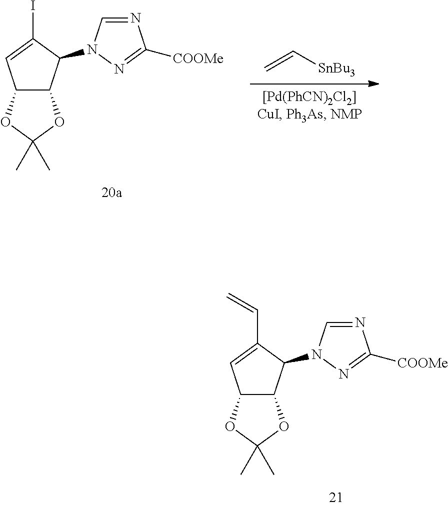

Iodide 20a (478 mg, 1.22 mmol, 1.00 eq.) was dissolved in NMP (4 mL, freshly distilled) and [Pd(PhCN)₂Cl₂] (14.1 mg, 36.7 μmol, 3 mol %), CuI (14.0 mg, 73.3 μmol, 6 mol %) and Ph₃As (22.5 mg, 73.3 μmol, 6 mol %) was added. Then at 0° C. vinyl tributyltin (430 μL, 1.47 mmol, 1.20 eq.) was added dropwise. The reaction was stirred at this temperature for 3 h. The reaction was quenched with sat. NaHCO₃ solution (10 mL) and EtOAc (10 mL). After the separation of the organic phase, the aqueous phase was extracted with EtOAc (3×10 mL). The combined organic phases were washed with brine (15 mL), dried over MgSO₄ and concentrated in vacuo. Purification by column chromatography (SiO₂, Hex:EtOAc 2:1 to 1:1) gave 21 as a white solid (349 mg, 1.20 mmol, 98%). R_{f}=0.29 (Hex:EtOAc 1:1); 1H-NMR (400 MHz, CDCl₃) δ [ppm]=8.07 (s, 1H), 6.47 (dd, J=11.2, 17.6 Hz, 1H), 6.21 (s, 1H), 5.68 (s, 1H), 5.42 (d, J=5.4 Hz, 1H), 5.23 (d, J=17.7 Hz, 1H), 4.70 (d, J=5.6 Hz, 1H), 3.99 (s, 3H), 1.42 (s, 3H), 1.33 (s, 3H); ¹³C{¹H}-NMR (101 MHz, CDCl₃) δ [ppm]=160.1, 155.6, 143.9, 138.8, 136.5, 130.0, 119.7, 112.4, 84.4, 83.4, 69.4, 53.0, 27.5, 26.0; HRMS (ESI, 3.5 kV) calc for [M+Na]⁺ C₁₄H₁₇N₃O₄Na⁺ 314.11113, found 314.11088.

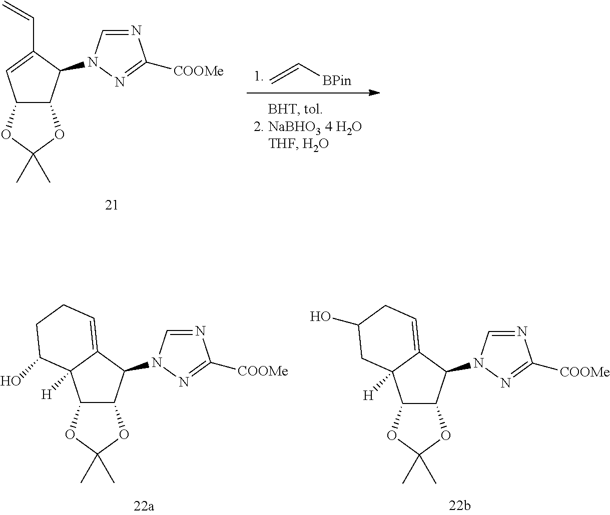

Diene 21 (320 mg, 1.10 mmol, 1.00 eq.) was dissolved in toluene (freshly distilled over CaH₂, 15 mL) and BHT (29.0 mg, 0.11 mmol, 10 mol %) and vinyl boronic acid pinacol ester (477 mL, 2.82 mmol, 2.50 eq.) were added. The reaction was heated to 135° C. (oil bath temperature) in a sealed tube for 96 h. Then the volatiles was removed and the crude mixture was dissolved in THF/H₂O (20 mL, 1:1). Then, NaBO₃×4 H₂O (866 mg, 3.56 mmol, 3.00 eq.) was added. After 90 min, the reaction was completed and diluted with EtOAc/brine (1:1, 20 mL). After the separation of the organic phase, the aqueous phase was extracted with EtOAc (3×20 mL). The combined organic phases were dried over MgSO₄ and concentrated in vacuo. Purification by column chromatography (SiO₂, DCM:EtOAc 1:1 to EtOAc/DCM 1:3 to pure EtOAc) gave 2a as a white solid (114 mg, 0.34 mmol, 31%) and 22b (179 mg, 0.53 mmol, 49%) as a mixture of isomers. 22a: R_{f}=0.30 (EtOAc:DCM 1:1); ¹H-NMR (400 MHz, CDCl₃) δ [ppm]=8.25 (s, 1H), 5.20 (brs, 1H), 5.06 (dd, J=5.6, 7.7 Hz, 1H), 4.92 (m, 1H), 4.64 (dd, J=5.0, 7.7 Hz, 1H), 4.00 (s, 3H), 3.69 (m, 1H), 2.63 (m, 1H), 2.11-2.20 (m, 2H), 1.95-2.05 (m, 2H), 1.52-1.65 (m, 1H), 1.56 (s, 3H), 1.33 (s, 3H); ¹³C{¹H}-NMR (101 MHz, CDCl₃) δ [ppm]=160.2, 155.6, 145.7, 136.4, 122.5, 114.5, 83.3, 82.3, 71.5, 68.6, 53.0, 52.5, 31.3, 27.3, 25.04, 24.97; HRMS (ESI, 3.5 kV) calc for [M+Na]⁺ C₁₆H₂₁N₃O₅Na⁺ 358.13734, found 358.13714. 22b: R_{f}=0.16 (EtOAc:DCM 1:1).

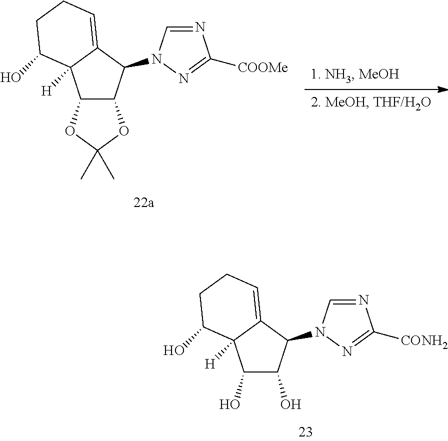 [SMILES: COC(=O)c1ncn([C@@H]2C3=CCC[C@@H](O)[C@@H]3[C@H]3OC(C)(C)O[C@H]32)n1 ; "1. NH3, MeOH" ; "2. MeOH, THF/H2O" ; NC(=O)c1ncn([C@@H]2C3=CCC[C@@H](O)[C@@H]3[C@@H](O)[C@H]2O)n1]

A solution of 22a (26.0 mg, 77.5 mmol) in MeOH (0.5 mL) was prepared and NH₃ (7M in MeOH, 0.5 mL) was added. The solution was stirred for 16 h and the volatiles was removed. Then after the crude was dissolved in MeOH (0.5 mL), TFA (10% in H₂O, 0.5 mL) was added and the reaction was stirred for another 23 h. Removal of the volatiles was followed by purification by column chromatography (SiO₂, EtOAc 5% to 10% MeOH) yielded nucleoside analogue 23 (17.8 mg, 63.5 mmol, 82% over 2 steps) as a white solid. R_{f}=0.08 (EtOAc, 5% MeOH); [α]_{D}²⁵=−37.1 (c=0.10, MeOH); ¹H-NMR (400 MHz, MeOD-d4) δ [ppm]=8.60 (s, 1H), 5.25 (brs, 1H), 5.03 (m, 1H), 4.25 (dd, J=6.2, 6.8 Hz, 1H), 4.16 (dd, J=5.0, 5.6 Hz, 1H), 3.57 (ddd, J=3.8, 9.7, 11.3 Hz, 1H), 2.48 (m, 1H), 2.08-2.28 (m, 2H), 1.91-2.00 (m, 1H), 1.57 (ddt, J=7.3, 11.3, 11.8 Hz, 1H); ³C{¹H}-NMR (101 MHz, CDCl₃) δ [ppm]=163.5, 158.1, 147.1, 135.9, 122.7, 76.6, 73.8, 71.8, 68.6, 53.7, 32.3, 26.1; HRMS (ESI, 3.5 kV) calc for [M+Na]⁺ C₁₂H₁₆N₄O₄Na⁺ 303.10638, found 303.10631.

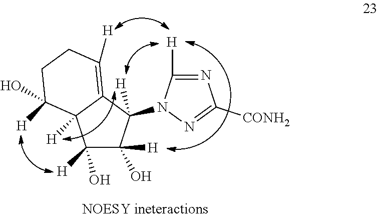 [SMILES: NC(=O)c1ncn([C@@H]2C3=CCC[C@@H](O)[C@@H]3[C@@H](O)[C@H]2O)n1]

### Example 7. Preparation of (1R,2S,3R,3aS,4R)-1-(3-carbamoyl-1H-1,2,4-triazol-1-yl)-2,3-dihydroxy-2,3,3a,4,5,6-hexahydro-1H-inden-4-yl isobutyrate (24)

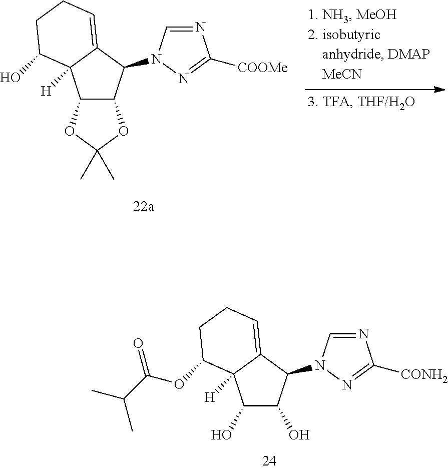 [SMILES: COC(=O)c1ncn([C@@H]2C3=CCC[C@@H](O)[C@@H]3[C@H]3OC(C)(C)O[C@H]32)n1 ; CC(C)C(=O)O[C@@H]1CCC=C2[C@H]1[C@@H](O)[C@@H](O)[C@@H]2n1cnc(C(N)=O)n1]

Alcohol 22a (32 mg, 95.5 μmol, 1.00 eq.) was dissolved in MeOH (0.35 mL) and NH₃ (7M in MeOH, 0.35 mL) was added. After 16 h, the solvent was removed in vacuo. The residue was dissolved in dry ACN (1.5 mL) and at 0° C. DMAP (14.6 mg, 0.12 mmol, 1.25 eq.) and isobutyric anhydride (22.7 mg, 24 μL, 1.50 eq.) were added. After 20 min, the reaction was quenched with sat. NaHCO₃ (5 mL) and ethyl acetate (5 mL). The organic phase was separated, and the aqueous phase was extracted with ethyl acetate (4×5 mL). The combined organic phases were dried over MgSO₄, and the solvent was removed in vacuo. Purification by column chromatography (SiO₂, DCM:EtOAc 3:1) gave the butyric ester which was directly deprotected by redissolving in MeOH (0.5 mL) and addition of TFA (10% in H₂O, 0.5 mL). After 24 h the solvent was removed in vacuo. Purification by column chromatography (SiO₂, EtOAc 5% MeOH) gave nucleoside analogue 24 (18.6 mg, 53.1 μmol, 55% over 3 steps) as a white solid. R_{f}=0.33 (EtOAc 5% MeOH); [α]_{D}²⁵=−55.4 (c=0.10, MeOH); ¹H-NMR (400 MHz, CDCl₃) δ [ppm]=8.63 (s, 1H), 5.25 (s, 1H), 5.17 (m, 1H), 4.91 (m, 1H), 4.22 (dd, J=5.6, 5.8 Hz, 1H), 4.08 (dd, J=5.9, 6.0 Hz, 1H), 2.74 (m, 1H), 2.61 (sept, J=6.9 Hz, 1H), 2.14-2.33 (m, 2H), 1.93-2.03 (m, 1H), 1.68 (ddt, J=7.3, 11.4, 11.4 Hz, 1H), 1.21 (d, J=7.2 Hz, 3H), 1.91 (d, J=7.1 Hz, 3H); ¹³C{¹H}-NMR (101 MHz, CDCl₃) δ [ppm]=178.7, 157.6, #146.9*, 135.6, 123.8, 76.7, 74.4, 74.3, 68.5, 50.5, 35.4, 28.9, 25.8, 19.3 (2×); HRMS (ESI, 3.5 kV) calc for [M+Na]⁺ C₁₅H₂₂N₄O₅Na⁺ 373.14824, found 373.14822. #found by HMBC, *found by HSQC, note: carbamoyl carbon was not observed.

### Example 8. Preparation of 1-((1R,2S,3R,3aS,5R)-2,3,5-trihydroxy-2,3,3a,4,5,6-hexahydro-1H-inden-1-yl)-1H-1,2,4-triazole-3-carboxamide (26)

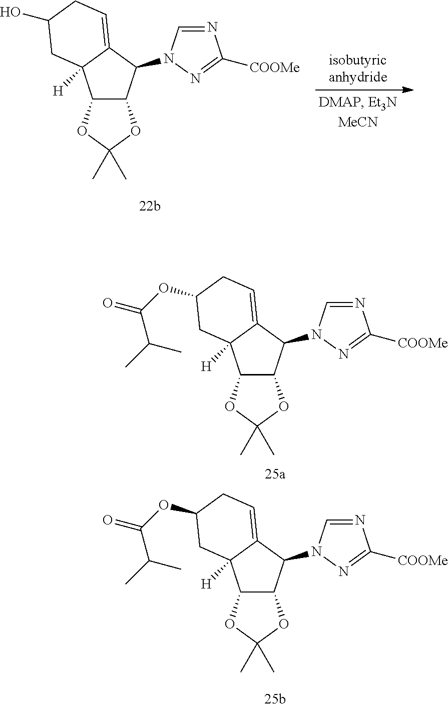

Diastereomeric mixture 22b (175.5 mg, 0.52 mmol, 1.00 eq.) was dissolved in MeCN (6 mL) and isobutyric anhydride (173 μL, 1.04 mmol, 2.00 eq.) Et₃N (145 μL, 1.04 mmol, 2.00 eq.) and DMAP (6.4 mg, 52.2 μmol, 10 mol %) were added subsequently. After 18 h, the reaction was quenched by the addition of sat. NaHCO₃ solution and diluted with EtOAc. The organic phase was separated, and the aqueous phase was extracted with ethyl acetate (3×10 mL). The combined organic phases were washed with brine (15 mL) dried over MgSO₄, and the solvent was removed in vacuo. Purification by column chromatography (SiO₂, Hex:EtOAc 1:1) gave 25a (48.1 mg, 11.8 μmol, 23%) and 25b (108.8 mg, 26.8 μmol, 52%) both as white solids. 25a: R_{f}=0.49 (Hex:EtOAc 1:2); 1H-NMR (600 MHz, CDCl₃) δ [ppm]=8.26 (s, 1H), 5.25 (brs, 1H), 5.21 (brs, 1H), 5.08 (dd, J=5.4, 7.3 Hz, 1H), 4.88 (m, 1H), 4.43 (dd, J=7.4, 9.3 Hz, 1H), 3.99 (s, 3H), 2.27 (brs, 1H), 2.50 (hep, J=7.0 Hz, 1H), 2.44 (ddd, J=4.0, 5.3, 13.6 Hz, 1H), 2.32-2.35 (m, 1H), 2.09 (d, J=19.1 Hz, 1H), 1.55 (s, 3H), 1.46 (ddd, J=2.2, 11.9, 13.6 Hz, 1H), 1.31 (s, 3H), 1.14 (d, J=7.0 Hz, 3H), 1.12 (d, J=7.0 Hz, 3H); ¹³C{¹H}-NMR (151 MHz, CDCl₃) δ [ppm]=176.6, 160.2, 155.7, 145.7, 138.4, 119.1, 114.2, 83.5, 83.3, 68.5, 66.7, 53.0, 39.3, 34.2, 30.5, 29.8, 27.4, 25.1, 19.12, 19.05; HRMS (ESI, 3.5 kV) calc for [M+Na]⁺ C₂₀H₂₇N₃O₆Na⁺ 428.17921, found 428.17857. 25b: R_{f}=0.56 (Hex:EtOAc 1:2); 1H-NMR (600 MHz, CDCl₃) δ [ppm]=8.25 (s, 1H), 5.19 (brs, 1H), 5.05 (ddd, J=1.0, 5.1, 7.2 Hz, 1H), 4.95 (m, 1H), 4.91 (m, 1H), 4.41 (ddd, J=0.9, 5.6, 7.1 Hz, 1H), 3.99 (s, 3H), 2.80 (brs, 1H), 2.50 (hep, J=7.0 Hz, 1H), 2.35-2.43 (m, 2H), 1.94-2.03 (m, 1H), 1.51 (m, 3H), 1.47 (q, J=12.0 Hz, 1H), 1.29 (s, 3H), 1.13 (m, 6H); ¹³C{¹H}-NMR (151 MHz, CDCl₃) δ [ppm]=176.7, 160.2, 155.7, 145.7, 138.5, 120.5, 114.1, 83.5, 82.8, 68.9, 67.9, 53.0, 44.2, 34.1, 32.4, 30.2, 27.4, 25.1, 19.04, 19.02; HRMS (ESI, 3.5 kV) calc for [M+Na]⁺ C₂₀H₂₇N₃O₆Na⁺ 428.17921, found 428.17857.

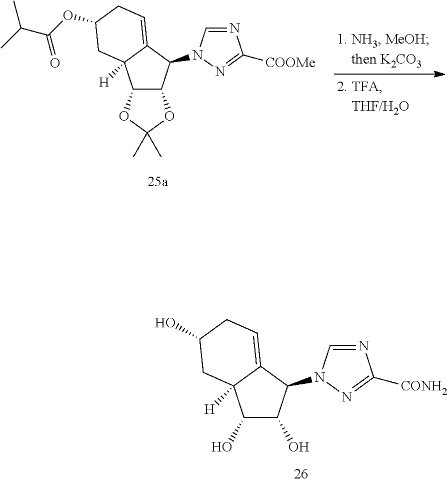 [SMILES: COC(=O)c1ncn([C@@H]2C3=CC[C@@H](OC(=O)C(C)C)C[C@@H]3[C@H]3OC(C)(C)O[C@H]32)n1 ; NC(=O)c1ncn([C@@H]2C3=CC[C@@H](O)C[C@@H]3[C@@H](O)[C@H]2O)n1]

Ester 25a (24.8 mg, 61.2 μmol, 1.00 eq.) was dissolved in MeOH (0.5 mL) and NH₃ (7M in MeOH, 0.5 mL) was added. The reaction was stirred for 16 h and then K₂CO₃ (16.9 mg, 0.12 mmol, 2.00 eq.) was added. After further 16 h stirring, a brine/EtOAc (10 mL, 1:1) mixture was added. The organic phase was separated, and the aqueous phase was extracted with EtOAc (4×4 mL). The combined organic phases were dried over MgSO₄ and the solvent was removed in vacuo. The crude was redissolved in MeOH (0.5 mL) and TFA (10% in H₂O, 0.5 mL) was added. The reaction was stirred for 24 h, then the solvent was removed in vacuo. Purification by column chromatography (SiO₂, EtOAc 7% MeOH) gave nucleoside analogue 26 (16.0 mg, 57.1 μmol, 93% over 3 steps) as a white solid. R_{f}=0.21 (EtOAc, 5% MeOH); [α]_{D}²⁵=+6.2 (c=0.12, MeOH); ¹H-NMR (400 MHz, MeOD-d4) δ [ppm]=8.60 (s, 1H), 5.24 (brs, 1H), 5.11 (m, 1H), 4.25 (dd, J=4.1, 5.7 Hz, 1H), 4.18 (m, 1H), 3.96 (dd, J=5.7, 8.0 Hz, 1H), 2.80 (brs, 1H), 2.23-2.34 (m, 1H), 2.09 (d, J=19.1 Hz, 1H), 1.40 (ddd, J=1.8, 11.8, 13.3 Hz, 1H); ¹³C{¹H}-NMR (101 MHz, MeOD-d4) δ [ppm]=163.5, 158.2, 148.9, 138.1, 121.2, 77.1, 76.9, 69.1, 65.0, 39.3, 34.2, 34.0; HRMS (ESI, 3.5 kV) calc for [M+H]⁺ C₁₂H₁₆N₄O₄H⁺ 281.12443, found 281.12393.

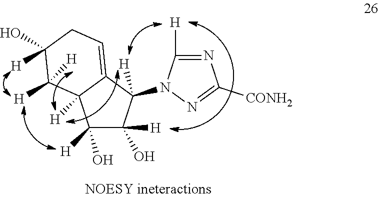

### Example 9. Preparation of (1R,2S,3R,3aS,5R)-1-(3-carbamoyl-1H-1,2,4-triazol-1-yl)-2,3-dihydroxy-2,3,3a,4,5,6-hexahydro-1H-inden-5-yl isobutyrate (27)

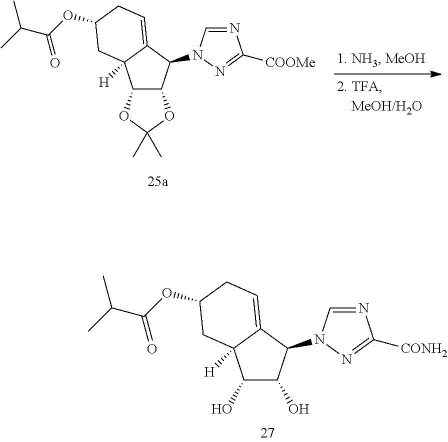 [SMILES: COC(=O)c1ncn([C@@H]2C3=CC[C@@H](OC(=O)C(C)C)C[C@@H]3[C@H]3OC(C)(C)O[C@H]32)n1 ; CC(C)C(=O)O[C@@H]1CC=C2[C@@H](n3cnc(C(N)=O)n3)[C@H](O)[C@H](O)[C@H]2C1]

Ester 25a (20.1 mg, 49.6 μmol) was dissolved in MeOH (0.5 mL) and NH₃ (7M in MeOH, 0.5 mL) was added. The reaction was stirred for 16 h and then the volatiles was removed in vacuo. The crude was redissolved in MeOH (0.75 mL) and TFA (10% in H₂O, 0.75 mL) was added. After 24 h the solvent was removed in vacuo. Purification by column chromatography (SiO₂, EtOAc 5% MeOH) gave nucleoside analogue 27c (16.2 mg, 46.2 μmol, 93% over 2 steps) as a white solid. R_{f}=0.17 (EtOAc, 5% MeOH); [α]⁵=+3.2 (c=0.12, MeOH); ¹H-NMR (400 MHz, CDCl₃) δ [ppm]=8.70 (s, 1H), 5.28 (brs, 1H), 5.23 (brs, 1H), 5.17 (brs, 1H), 4.26 (dd, J=4.3, 5.1 Hz, 1H), 3.99 (dd, J=5.8, 8.0 Hz, 1H), 2.77 (brs, 1H), 2.52 (sept, J=7.0 Hz, 1H), 2.31-2.45 (m, 2H), 2.16 (d, J=19.3 Hz, 1H), 1.48 (t, J=12.5 Hz, 1H), 1.16 (d, J=7.0 Hz, 3H, 1.13 (d, J=7.0 Hz, 3H); ¹³C{¹H}-NMR (101 MHz, CDCl₃) δ [ppm]=#178.3, 138.3, 121.1, 77.1, 77.0, 69.0, 68.8, 39.7, 35.4, 31.4, 19.34, 19.26; HRMS (ESI, 3.5 kV) calc for C₁₆H₂₂N₄O₅Na⁺ [M+Na]+373.14824, found 373.14763. # not all carbon signals could be found due to broad signals.

### Example 10. Preparation of 1-((1R,2S,3R,3aS,5S)-2,3,5-trihydroxy-2,3,3a,4,5,6-hexahydro-1H-inden-1-yl)-1H-1,2,4-triazole-3-carboxamide (28)

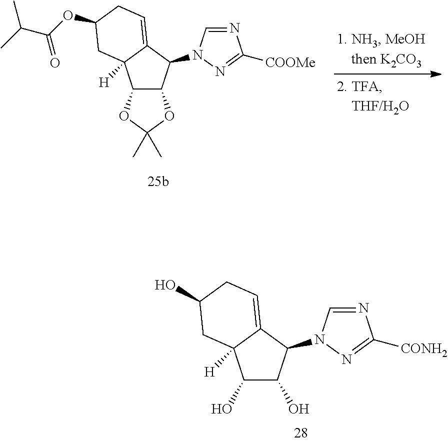 [SMILES: COC(=O)c1ncn([C@@H]2C3=CC[C@H](OC(=O)C(C)C)C[C@@H]3[C@H]3OC(C)(C)O[C@H]32)n1 ; NC(=O)c1ncn([C@@H]2C3=CC[C@H](O)C[C@@H]3[C@@H](O)[C@H]2O)n1]

Ester 25b (21.6 mg, 53.3 μmol, 1.00 eq.) was dissolved in MeOH (0.5 mL) and NH₃ (7M in MeOH, 0.5 mL) was added. The reaction was stirred for 17.5 h and then K₂CO₃ (14.7 mg, 0.11 mmol, 2.00 eq.) was added. After a further 24 h, H₂O/EtOAc (10 mL, 1:1) was added. The organic phase was separated and the aqueous phase was extracted with EtOAc (4×4 mL). The combined organic phases were dried over MgSO₄ and the solvent was removed in vacuo. The crude was redissolved in MeOH (0.5 mL) and TFA (10% in H₂O, 0.5 mL) was added. The reaction was stirred for 27 h, then the solvent was removed in vacuo. Purification by column chromatography (SiO₂, EtOAc 7.5% MeOH) gave nucleoside analogue 28 (10.8 mg, 38.5 μmol, 72% over 3 steps) as a white solid. R_{f}=0.08 (EtOAc, 7.5% MeOH); [α]_{D}²⁵=+4.9 (c=0.13, MeOH); 1H-NMR (400 MHz, MeOD-d4) δ [ppm]=8.59 (s, 1H), 5.19 (s, 1H), 5.16 (m, 1H), 4.24 (dd, J=3.8, 5.5 Hz, 1H), 3.95 (dd, J=5.9, 8.3 Hz, 1H), 3.90 (m, 1H), 2.70 (m, 1H), 2.37-2.46 (m, 1H), 2.34 (ddd, J=3.6, 5.2, 11.6 Hz, 1H), 1.83-1.94 (m, 1H), 1.34 (q, J=11.6 Hz, 1H); ¹³C{¹H}-NMR (101 MHz, CDCl₃) δ [ppm]=163.5, 158.3, 146.9, 138.3, 122.6, 77.5, 76.6, 68.6, 68.2, 45.3, 37.2, 35.4; HRMS (ESI, 3.5 kV) calc for [M+H]+C₁₂H₁₆N₄O₄H⁺ 281.12375, found 281.12443.

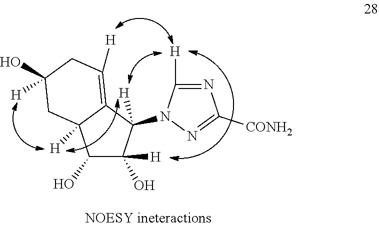 [SMILES: NC(=O)c1ncn([C@@H]2C3=CC[C@H](O)C[C@@H]3[C@@H](O)[C@H]2O)n1]

### Example 11. Preparation of (1R,2S,3R,3aS,5S)-1-(3-carbamoyl-1H-1,2,4-triazol-1-yl)-2,3-dihydroxy-2,3,3a,4,5,6-hexahydro-1H-inden-5-yl isobutyrate (29)

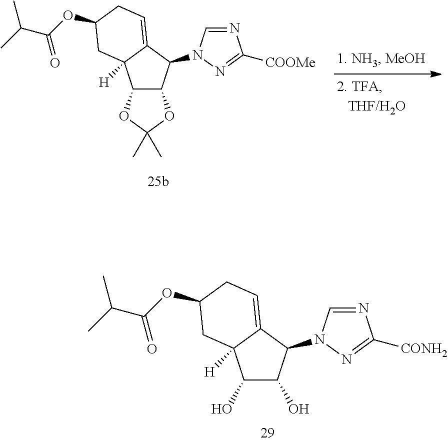 [SMILES: COC(=O)c1ncn([C@@H]2C3=CC[C@H](OC(=O)C(C)C)C[C@@H]3[C@H]3OC(C)(C)O[C@H]32)n1 ; CC(C)C(=O)O[C@H]1CC=C2[C@@H](n3cnc(C(N)=O)n3)[C@H](O)[C@H](O)[C@H]2C1]

Ester 25b (20.8 mg, 51.3 μmol) was dissolved in MeOH (0.5 mL) and NH₃ (7M in MeOH, 0.5 mL) was added. The reaction was stirred for 17.5 h and then the volatiles were removed in vacuo. The crude was redissolved in MeOH (0.5 mL) and TFA (10% in H₂O, 0.5 mL) was added. After 24 h the solvent was removed in vacuo. Purification by column chromatography (SiO₂, EtOAc 3% MeOH) gave nucleoside analogue 29 (15.7 mg, 44.8 μmol, 87% over 2 steps) as a white solid. R_{f}=0.37 (EtOAc, 5% MeOH); [α]5=+5.9 (c=0.09, MeOH); ¹H-NMR (600 MHz, CDCl₃) δ [ppm]=8.61 (s, 1H), 5.22 (brs, 1H), 5.19 (m, 1H), 5.00 (dddd, J=3.8, 6.4, 9.7, 12.3, 1H), 4.24 (dd, J=3.5, 5.6 Hz, 1H), 3.97 (dd, J=5.7, 8.4 Hz, 1H), 2.77 (brs, 1H), 2.54 (hep, J=7.0 Hz, 1H), 2.46-2.51 (m, 1H), 2.37 (ddd, J=3.5, 5.4, 11.4 Hz, 1H), 1.99-2.06 (m, 1H), 1.47 (q, J=11.5 Hz, 1H), 1.15 (d, J=7.0 Hz, 3H), 1.14 (d, J=7.0 Hz, 3H); ¹³C{¹H}-NMR (151 MHz, CDCl₃) δ [ppm]=178.4, 163.4, 158.3, 147.0, 138.5, 122.1, 77.5, 76.6, 71.1, 68.9, 44.6, 35.3, 33.4, 31.9, 19.3 (2×); HRMS (ESI, 3.5 kV) calc for [M+Na]⁺ C₁₆H₂₂N₄O₅Na⁺ 373.14824, found 373.14768.

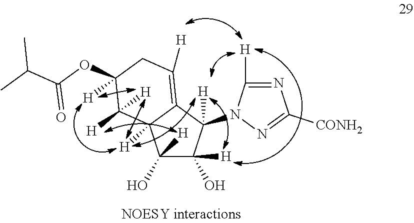

### Example 12. Preparation 1-((1R,2S,3R,3aR,4R)-2,3,4-trihydroxy-2,3,3a,4,5,6-hexahydro-1H-inden-1-yl)-1H-1,2,4-triazole-5-carboxamide (30)

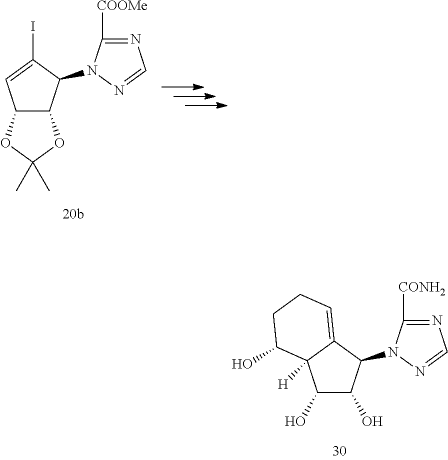 [SMILES: COC(=O)c1ncnn1[C@@H]1C(I)=C[C@H]2OC(C)(C)O[C@@H]12 ; NC(=O)c1ncnn1[C@@H]1C2=CCC[C@@H](O)[C@@H]2[C@@H](O)[C@H]1O]

Nucleoside analogue 30 was synthesized using the described methods in example 6 using intermediate 20b. R_{f}=0.23 (EtOAc/MeOH 3%); [α]5=−64.5 (c=0.15, MeOH)¹H-NMR (400 MHz, CDCl₃) δ [ppm]=7.98 (s, 1H), 6.28 (s, 1H), 4.91 (m, 1H), 4.33 (dd, J=5.7, 5.7 Hz, 1H), 4.19 (dd, J=5.9, 6.0 Hz, 1H), 3.56 (ddd, J=3.7, 9.2, 11.4 Hz, 1H), 2.51 (brs, 1H), 2.14-2.24 (m, 1H), 2.00-2.13 (m, 1H), 1.89-1.97 (m, 1H), 1.57 (ddt, J=6.9, 11.4, 11.5, 1H); ¹³C{¹H}-NMR (101 MHz, CDCl₃) δ [ppm]=161.0, 151.2, 148.8, 136.6, 122.3, 76.8, 75.2, 72.6, 68.1, 53.1, 32.3, 26.1; HRMS (ESI, 3.5 kV) calc for [M+Na]⁺ C₁₂H₁₆N₄O₄Na⁺ 303.10638, found 303.10612.

### Example 13. Preparation of (1R,2S,3R,3aS,4R)-1-(5-carbamoyl-1H-1,2,4-triazol-1-yl)-2,3-dihydroxy-2,3,3a,4,5,6-hexahydro-1H-inden-4-yl isobutyrate (31)

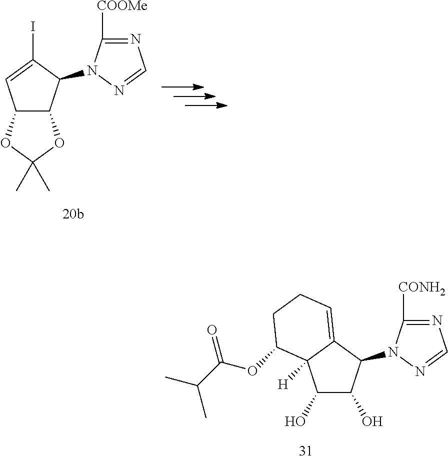 [SMILES: COC(=O)c1ncnn1[C@@H]1C(I)=C[C@H]2OC(C)(C)O[C@@H]12 ; CC(C)C(=O)O[C@@H]1CCC=C2[C@H]1[C@@H](O)[C@@H](O)[C@@H]2n1ncnc1C(N)=O]

Nucleoside analogue 31 was synthesized using the described methods in examples 6 and 7 using intermediate 20b. R_{f}=0.54 (EtOAc, 5% MeOH); [α]_{D}²⁵=−66.4 (c=0.10, MeOH); ¹H-NMR (400 MHz, CDCl₃) δ [ppm]=7.98 (s, 1H), 6.29 (s, 1H), 5.04 (m, 1H), 4.87 (m, 1H), 4.27 (dd, J=4.4, 5.5 Hz, 1H), 4.14 (dd, J=5.9, 7.4 Hz, 1H), 2.75 (m, 1H), 2.60 (sept, J=7.0 Hz, 1H), 2.19-2.29 (m, 1H), 2.07-2.19 (m, 1H), 1.89-1.98 (m, 1H), 1.67 (ddt, J=6.8, 11.6, 11.9 Hz, 1H), 1.20 (d, J=7.0 Hz, 3H), 1.18 (d, J=7.0 Hz, 3H); ¹³C{¹H}-NMR (101 MHz, CDCl₃) δ [ppm]=178.7, 161.0, 151.3, 148.5, 136.5, 123.2, 77.0, 75.7, 74.8, 67.9, 50.0, 35.4, 28.9, 25.8, 19.4, 19.3; HRMS (ESI, 3.5 kV) calc for [M+Na]⁺ C₁₆H₂₂N₄O₅Na⁺ 373.14824, found 373.14786.

### Example 14. Preparation of 1-((1R,2S,3R,3aS,5R)-2,3,5-trihydroxy-2,3,3a,4,5,6-hexahydro-1H-inden-1-yl)-1H-1,2,4-triazole-5-carboxamide (32)

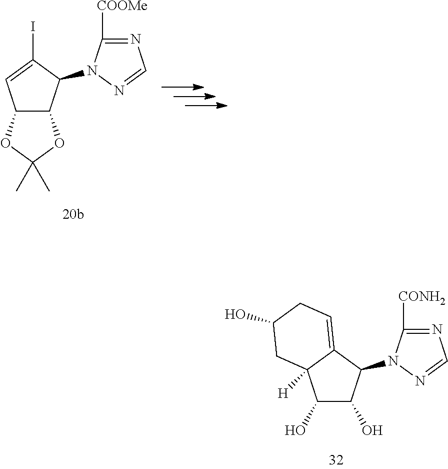 [SMILES: COC(=O)c1ncnn1[C@@H]1C(I)=C[C@H]2OC(C)(C)O[C@@H]12 ; NC(=O)c1ncnn1[C@@H]1C2=CC[C@@H](O)C[C@@H]2[C@@H](O)[C@H]1O]

Nucleoside analogue 32 was synthesized using the described methods in examples 6 and 8 using intermediate 20b. R_{f}=0.21 (EtOAc, 5% MeOH); [α]_{D}²⁵=−28.1 (c=0.14, MeOH); 1H-NMR (600 MHz, CDCl₃) δ [ppm]=7.95 (s, 1H), 6.29 (s, 1H), 4.97 (s, 1H), 4.32 (dd, J=3.3, 5.6 Hz, 1H), 4.15 (brs, 1H), 4.02 (dd, J=5.5, 8.9 Hz, 1H), 2.81 (brs, 1H), 2.28 (ddd, J=3.7, 5.3, 12.9 Hz, 1H), 2.17-2.24 (m, 1H), 2.07 (d, J=19.2 Hz, 1H), 1.34 (t, J=11.9 Hz, 1H); ¹³C{¹H}-NMR (151 MHz, CDCl₃) δ [ppm]=161.1, 151.3, 148.5, 138.8, 120.3, 77.8, 77.3, 68.5, 65.1, 39.1, 34.4, 30.0; HRMS (ESI, 3.5 kV) calc for [M+Na]⁺ C₁₂H₁₆N₄O₄Na⁺ 303.10638, found 303.10603.

### Example 15. Preparation of (1R,2S,3R,3aS,5R)-1-(5-carbamoyl-1H-1,2,4-triazol-1-yl)-2,3-dihydroxy-2,3,3a,4,5,6-hexahydro-1H-inden-5-yl isobutyrate (33)

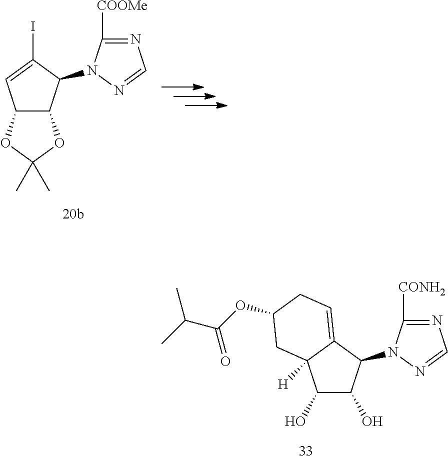 [SMILES: COC(=O)c1ncnn1[C@@H]1C(I)=C[C@H]2OC(C)(C)O[C@@H]12 ; CC(C)C(=O)O[C@@H]1CC=C2[C@@H](n3ncnc3C(N)=O)[C@H](O)[C@H](O)[C@H]2C1]

Nucleoside analogue 33 was synthesized using the described methods in examples 6 and 9 using intermediate 20b. R_{f}=0.53 (EtOAc, 7.5% MeOH); [α]_{D}²⁵=−30.3 (c=0.11, MeOH); 1H-NMR (400 MHz, MeOD-d4) δ [ppm]=7.96 (s, 1H), 6.31 (brs, 1H), 5.21 (brs, 1H), 5.03 (brs, 1H), 4.30 (dd, J=2.8, 5.5 Hz, 1H), 4.04 (dd, J=5.6, 9.2 Hz, 1H), 2.77 (brs, 1H), 2.54 (sept, J=7.0 Hz, 1H), 2.40 (ddd, J=4.5, 4.6, 13.3 Hz, 1H), 2.23-2.34 (m, 1H), 2.13 (d, J=19.7 Hz, 1H), 1.40 (ddd, J=1.5, 11.6, 13.2 Hz, 1H), 1.16 (d, J=7.2 Hz, 3H), 1.14 (d, J=7.2 Hz, 3H); ¹³C{¹H}-NMR (101 MHz, MeOD-d4) δ [ppm]=177.1, 159.8, 150.1, 147.1, 137.9, 119.0, 76.6, 76.1, 67.6, 67.1, 38.1, 34.1, 30.3, 30.2, 18.1, 18.0; HRMS (ESI, 3.5 kV) calc for [M+Na]⁺ C₁₆H₂₂N₄O₅Na⁺ 373.14824, found 373.14778.

### Example 16. Preparation of 1-((1R,2S,3R,3aS,5S)-2,3,5-trihydroxy-2,3,3a,4,5,6-hexahydro-1H-inden-1-yl)-1H-1,2,4-triazole-5-carboxamide (34)

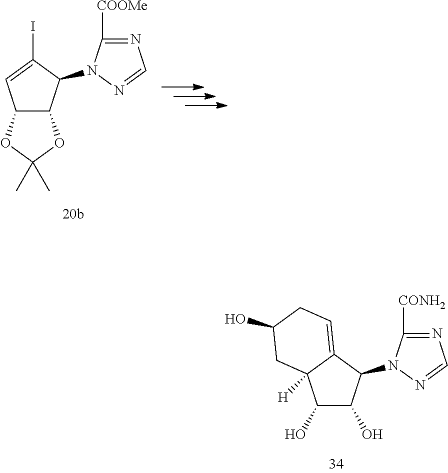 [SMILES: COC(=O)c1ncnn1[C@@H]1C(I)=C[C@H]2OC(C)(C)O[C@@H]12 ; NC(=O)c1ncnn1[C@@H]1C2=CC[C@H](O)C[C@@H]2[C@@H](O)[C@H]1O]

Nucleoside analogue 34 was synthesized using the described methods in examples 6 and 10 using intermediate 20b. R_{f}=0.35 (EtOAc, 5% MeOH); [α]_{D}²⁵=−84.6 (c=0.09, MeOH); ¹H-NMR (400 MHz, CDCl₃) δ [ppm]=7.96 (s, 1H), 6.23 (brs, 1H), 4.99 (m, 1H), 4.30 (dd, J=2.6, 5.6 Hz, 1H), 3.99 (dd, J=5.5, 9.2 Hz, 1H), 3.90 (m, 1H), 2.71 (brs, 1H), 2.28-2.44 (m, 2H), 1.81 (m, 1H), 1.27 (q, J=11.5 Hz, 1H); ¹³C{¹H}-NMR (101 MHz, CDCl₃) δ [ppm]=161.1, 151.3, 148.3, 139.1, 121.7, 77.8, 77.5, 68.2, 68.0, 45.1, 37.4, 35.5; HRMS (ESI, 3.5 kV) calc for [M+Na]⁺ C₁₂H₁₆N₄O₄Na⁺ 303.10638, found 303.10603.

### Example 17. Preparation of 1-((1R,2S,3R,3aS,5S)-2,3,5-trihydroxy-2,3,3a,4,5,6-hexahydro-1H-inden-1-yl)-1H-1,2,4-triazole-5-carboxamide (35)

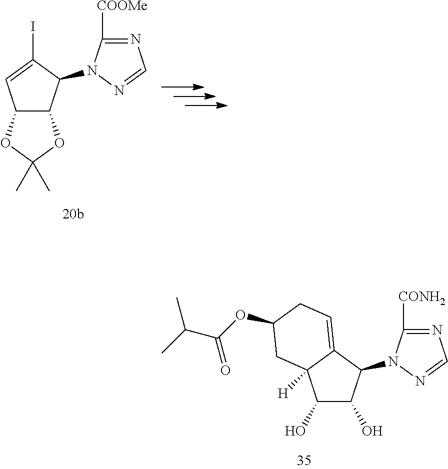 [SMILES: COC(=O)c1ncnn1[C@@H]1C(I)=C[C@H]2OC(C)(C)O[C@@H]12 ; CC(C)C(=O)O[C@H]1CC=C2[C@@H](n3ncnc3C(N)=O)[C@H](O)[C@H](O)[C@H]2C1]

Nucleoside analogue 35 was synthesized using the described methods in examples 6 and 11 using intermediate 20b. R_{f}=0.56 (EtOAc, 7.5% MeOH); [α]5=−42.9 (c=0.10, MeOH); ¹H-NMR (400 MHz, CDCl₃) δ [ppm]=7.97 (s, 1H), 6.27 (s, 1H), 5.04 (m, 1H), 4.94-5.02 (m, 1H), 4.33 (dd, J=2.4, 5.5 Hz, 1H), 4.03 (dd, J=5.4, 9.2 Hz, 1H), 2.78 (brs, 1H), 2.53 (sept, J=7.0 Hz, 1H), 2.41-2.50 (m, 1H), 2.37 (ddd, J=4.5, 5.0, 11.5 Hz, 1H), 1.89-2.00 (m, 1H), 1.40 (q, J=11.5 Hz, 1H), 1.14 (d, J=7.0 Hz, 3H), 1.13 (d, J=7.0 Hz, 3H); ¹³C{¹H}-NMR (101 MHz, CDCl₃) δ [ppm]=178.4, 161.0, 151.4, 148.4, 139.3, 121.2, 77.8, 77.4, 71.2, 68.0, 44.5, 35.3, 33.6, 31.9, 19.3; HRMS (ESI, 3.5 kV) calc for [M+Na]⁺ C₁₆H₂₂N₄O₅Na⁺ 373.14824, found 373.14765.

### Example 18. Preparation of 1-((1R,2S,3R,3aS)-2,3-dihydroxy-2,3,3a,4,5,6-hexahydro-1H-inden-1-yl)-1H-1,2,4-triazole-5-carboxamide (36)

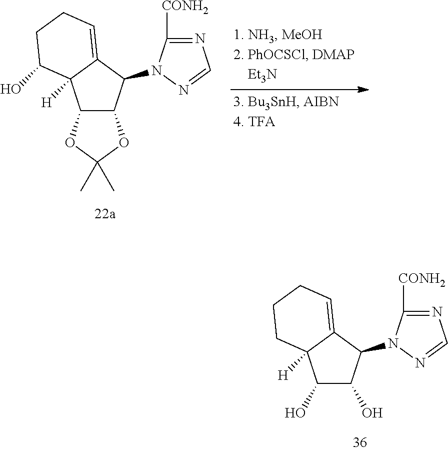 [SMILES: CC1(C)O[C@@H]2[C@H](O1)[C@@H]1C(=CCC[C@H]1O)[C@H]2n1ncnc1C(N)=O ; NC(=O)c1ncnn1[C@@H]1C2=CCCC[C@@H]2[C@@H](O)[C@H]1O]

Ester 22a (1.00 eq., 0.10 mmol, 35 mg) was dissolved in MeOH (1 mL) and NH₃ (7M in MeOH, 1 mL) was added. After 19 h the solvent was removed in vacuo and ACN (2 mL) was added. Then PhOCSCl (4.00 eq., 0.40 mmol, 72 mg, 58 μL) and DMAP (4.00 eq., 0.40 mmol, 51 mg) were added and the reaction was stirred for Id. Then sat. NaHCO₃ sol. (5 mL) was added and the aqueous phase was extracted with EtOAc (4×10 mL). The combined organic layers were dried over MgSO₄ and the solvent was removed in vacuo. The crude was purified using column chromatography (SiO₂, EA/DCM 1:2) yielding the thionocarbonate (28.3 mg, 62 μmol, 62%).

The product was dissolved in toluene (3 mL) and Bu₃SnH (4.00 eq., 0.24 mmol, 72.2 mg, 67 μL) and AIBN (0.25 eq., 15.5 μmol, 2.5 mg) were added, The reaction were stirred under reflux for 5 h. The mixture was directly separated by column chromatography (SiO₂, EA/DCM 1:2) and the resulting product was redissolved in MeOH (0.5 mL) and TFA (10% in H₂O, 0.5 mL) was added. After 22 h the solvent was removed, and the crude was subjected to column chromatography (SiO2, EA 5% MeOH) yielding deoxygenation 36 (18.4 mg). R_{f}=0.48 (EtOAc, 5% MeOH); ¹H-NMR (600 MHz, MeOD-d4) δ [ppm]=7.94 (s, 1H), 6.24 (s, 1H), 5.03 (s, 1H), 4.28 (dd, J=2.9, 5.5 Hz, 1H), 3.93 (dd, J=5.6, 8.8 Hz, 1H), 2.52 (brs, 1H), 2.17-2.23 (m, 1H), 2.00-2.08 (m, 1H), 1.81-1.95 (m, 2H), 1.47-1.56 (m, 1H), 1.12-1.21 (m, 1H); ¹³C{¹H}-NMR (150 MHz, MeOD-d4) δ [ppm]=161.1, 151.2, 148.4, 139.2, 123.4, 78.2, 77.3, 68.6, 44.7, 28.1, 25.8, 22.8.

### Example 19. Preparation of 1-((1R,2S,3R,3aR,4R)-2,3,4-trihydroxy-2,3,3a,4,5,6-hexahydro-1H-inden-1-yl)pyrimidine-2,4(1H,3H)-dione (42)

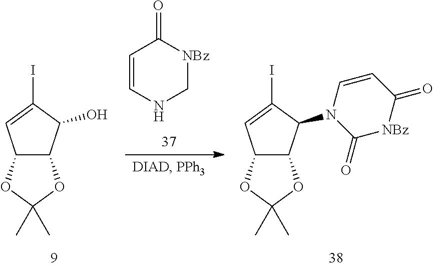

A solution of alcohol 9 (705 mg, 2.50 mmol, 1.00 eq.) and protected uracil 37 (649 mg, 3.00 mmol, 1.2 eq.) in THF (50 mL) was prepared and activated 4A MS MS was added and the mixture was stirred for 2 h. Then at 0° C. first PPh₃ (0.98 g, 3.75 mmol, 1.50 eq.) then DIAD (0.74 mL, 3.75 mmol, 1.50 eq.) were added. The reaction was stirred for 16 h. Then the reaction was filtered, and the solvent was removed in vacuo. Purification by column chromatography (SiO₂, Hex:EtOAc 4:1 to 1:1) gave 38 (0.96 g, 2.00 mmol, 80%) as a white foam. R_{f}=0.45 (Hex/EA 1:1); ¹H-NMR (400 MHz, CDCl₃) δ [ppm]=7.93 (d, J=7.5 Hz, 2H), 7.66 (tt, J=1.2, 7.5 Hz, 1H), 7.50 (t, J=7.9 Hz, 2H), 7.20 (brs, 1H), 6.53 (brs, 1H), 5.87 (d, J=8.0 Hz, 1H), 5.22 (d, J=5.7 Hz, 1H), 4.97 (brs, 1H), 4.55 (brs, 1H), 1.45 (s, 3H), 1.33 (s, 3H); ¹³C{¹H}-NMR (101 MHz, CDCl₃) δ [ppm]=168.3, 162.0, 149.1, 145.8, 135.5, 131.1, 130.7, 129.4, 112.7, 103.1, 85.6, 80.6, 27.1, 25.7; HRMS (ESI, 3.5 kV) calc for [M+Na]+C₁₉H₁₇IN₂O₅Na⁺ 503.00743, found 503.00744. Note: Broad signals were observed in the proton spectra as well as in the carbon spectra. Therefore, not all carbon signals could be found despite intensive 2D NMR experiments.

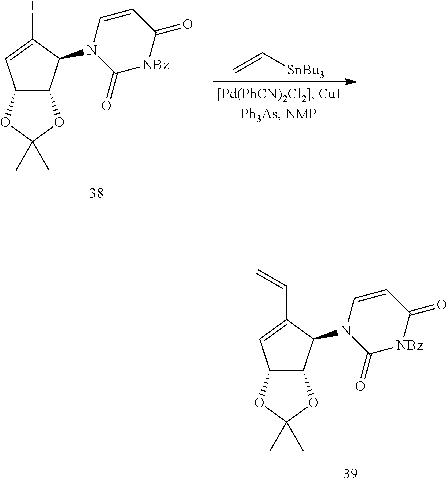

Vinyliodide 38 (1.70 g, 3.54 mmol, 1.00 eq.), [Pd(PhCN)₂Cl₂] (67.9 mg, 0.18 mmol, 5 mol %), CuI (67.4 mg, 0.35 mmol, 10 mol %) and Ph₃As (108.4 mg, 0.35 mmol, 10 mol %) were dissolved in freshly distilled NMP (over CaH₂, 11 mL). Then at 0° C. tributyl(vinyl)stannane (1.35 g, 1.24 mL, 4.25 mmol, 1.20 eq.) was added and the reaction was stirred for 4 h at this temperature. Then the reaction was quenched with half sat. NaHCO₃ solution (20 mL) and the aqueous phase was extracted with EtOAc (3×30 mL). The combined organic phase was washed with brine (30 mL) and brine was reextracted with EtOAc (1×15 mL). The combined organic phases were dried over MgSO₄ and concentrated to give the crude. The crude product was purified by column chromatography on silica gel (first column: Hex/EA 2:1 to 1:2, second column to remove residual NMP and impurities: DCM/EA 3:1). The diene 39 was isolated as a white crystalline solid (1.28 g, 3.36 mmol, 95%). R_{f}=0.53 (Hex/EA 1:2); 1H-NMR (400 MHz, CDCl₃) δ [ppm]=7.91 (d, J=7.3 Hz, 2H), 7.66 (tt, J=1.2, 7.5 Hz, 1H), 7.50 (t, J=8.0 Hz, 2H), 6.96 (brs, 1H), 6.47 (dd, J=11.0, 17.8 Hz, 1H), 6.19 (brs, 1H), 5.79 (d, J=8.1 Hz, 1H), 5.69 (brs, 1H), 5.39 (d, J=11.0 Hz, 1H), 5.34 (d, J=4.6 Hz, 1H), 5.25 (d, J=18.0 Hz, 1H), 4.63 (brs, 1H), 1.41 (s, 3H), 1.34 (s, 3H); ¹³C{¹H}-NMR (101 MHz, CDCl₃) δ [ppm]=168.5, 162.0, 149.8, 139.8, 136.7, 135.3, 131.5, 130.6, 130.0, 129.3, 120.3, 112.4, 102.8, 84.4, 83.3, 65.8, 27.4, 25.8; HRMS (ESI, 3.5 kV) calc for [M+Na]⁺ C₂₁H₂₀N₂O₅Na⁺ 403.12644, found 403.12626. Note: Broad signals were observed in the proton spectra as well as in the carbon spectra. Therefore, not all carbon signals could be found despite long measurement time.

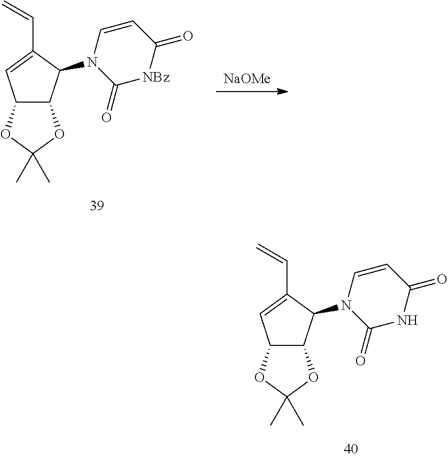

To a solution of ester 39 (1.23 g, 3.24 mmol, 1.00 equiv.) in dry MeOH (32 mL) was added NaOMe (0.52 g, 9.71 mmol, 3.00 equiv.) at 0° C. The reaction was stirred at room temperature for 21 h, then sat. NH₄Cl solution was added (50 mL). The mixture was extracted with EtOAc (4×50 mL), and the combined organic phases dried over MgSO₄ and concentrated in vacuo. Purification by column chromatography (SiO₂, 2:1 to 1:2 DCM/EtOAc) delivered deprotected 40 as a white solid (0.88 g, 3.18 mmol, 98%). R_{f}=0.19 (DCM/EA 3:1); 1H-NMR (400 MHz, CDCl₃) δ [ppm]=9.17 (s, 1H), 6.84 (brs, 1H), 6.43 (dd, J=11.0, 17.6 Hz, 1H), 6.17 (s, 1H), 5.74 (brs, 1H), 5.68 (dd, J=2.1, 8.0 Hz, 1H), 5.31 (m, 2H), 5.20 (d, J=17.8 Hz, 1H), 4.56 (d, J=3.9 Hz, 1H), 1.43 (s, 3H), 1.34 (s, 3H); ¹³C{¹H}-NMR (101 MHz, CDCl₃) δ [ppm]=163.3, 150.8, 139.9 (2×), 136.4, 129.8, 120.6, 112.3, 102.8, 84.5, 83.2, 65.1, 27.4, 25.8; HRMS (ESI, 3.5 kV) calc for [M+Na]⁺ C₁₄H₁₆N₂O₄Na⁺ 299.10023, found 299.09993.

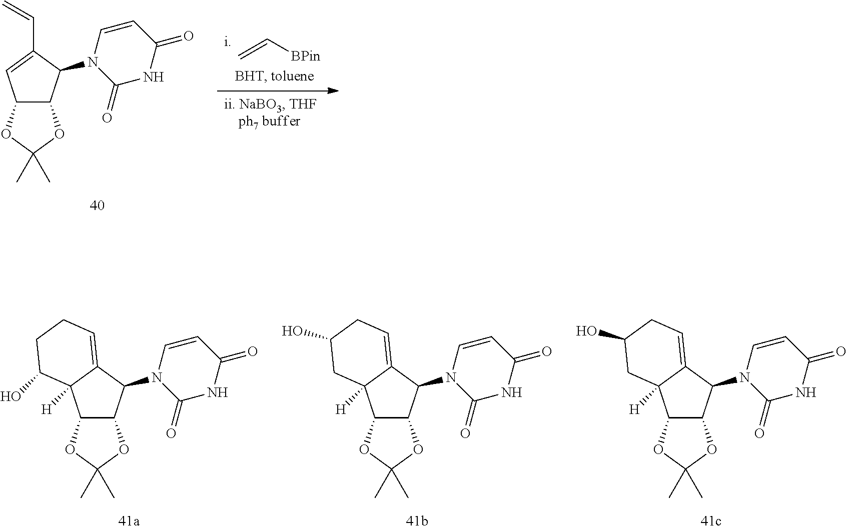

To a solution of diene 40 (227 mg, 0.82 mmol, 1.00 equiv.) in toluene (8 mL) was added BHT and vinyl pinacol borate (0.70 mL, 4.11 mmol, 5.00 equiv.). The reaction was stirred in a sealed tube for 4 d at 140° C. (oil bath temperature). Then the volatiles were removed and the crude was dissolved in THF/ph 7 buffer (16 mL, 1:1). Then NaBO₃ 4H₂O (505 mg, 3.29 mmol, 4.00 equiv.) was added and the mixture was stirred for 1.5 h. The reaction was then quenched by sat. Na₂S₂O₃ solution (10 mL). After phase separation, the aqueous phase was extracted with EtOAc (3×15 mL). The combined organic phases were dried over MgSO₄ and concentrated in vacuo. Purification by column chromatography (SiO₂, DCM:EtOAc 1:1 to DCM:acetone 4:1 to 1:1) gave 41a (64.3 mg, 0.20 mmol, 24%), 41b (21.0 mg, 65.6 μmol, 8%) and 41c (79.0 mg, 0.25 mmol, 30%) as white powders. 41a: R_{f}=0.23 (DCM/acetone 3:1); ¹H-NMR (600 MHz, 315 K, CDCl₃) δ [ppm]=8.59 (s, 1H), 7.08 (brs, 1H), 5.73 (d, J=6.0 Hz, 1H), 5.58 (brs, 0.3H), 5.27 (s, 1H), 4.21-5.10 (brs, 2.7H), 3.66 (brs, 1H), 2.55 (brs, 1H), 2.13-2.31 (m, 2H), 1.91-2.03 (m, 2H), 1.57 (s, 3H), 1.34 (s, 3H), 1.25-1.29 (m, 1H); ¹³C{¹H}-NMR (150 MHz, 315 K, CDCl₃) δ [ppm]=121.2, 114.8, 102.7, 83.1, 71.8, 53.5, 31.1, 27.4, 25.2, 24.8; HRMS (ESI, 3.5 kV) calc for [M+Na]⁺ C₁₆H₂₀N₂O₅Na⁺ 343.12644, found 343.12623. 41b: R_{f}=0.19 (DCM/acetone 3:1); ¹H-NMR (600 MHz, 315 K, CDCl₃) δ [ppm]=8.97 (s, 1H), 7.10 (brs, 1H), 5.72 (d, J=7.0 Hz, 1H), 5.23 (s, 1H), 4.30-5.10 (brs, 3H), 4.25 (s, 1H), 2.78 (brs, 1H), 2.29-2.38 (m, 2H), 2.09-2.19 (m, 1H), 1.55 (s, 3H), 1.43 (m, 1H), 1.33 (s, 3H); ¹³C{¹H}-NMR (150 MHz, 315 K, CDCl₃) δ [ppm]=140.6, 139.0, 117.9, 113.4, 102.3, 83.8 (2×), 64.4, 39.7, 33.5, 33.0, 27.8, 25.6; HRMS (ESI, 3.5 kV) calc for [M+Na]⁺ C₁₆H₂₀N₂O₅Na⁺ 343.12644, found 343.12638. 41c: R_{f}=0.16 (DCM/acetone 3:1); ¹H-NMR (600 MHz, 315 K, CDCl₃) δ [ppm]=9.18 (brs, 1H), 7.08 (brs, 1H), 5.74 (d, J=7.4 Hz, 1H), 5.25 (s, 1H), 4.21-5.11 (brs, 3H), 3.97 (brs, 1H), 2.67 (brs, 1H), 2.35-2.50 (m, 2H), 1.93-2.01 (m, 2H), 1.54 (s, 3H), 1.40-1.47 (m, 1H), 1.31 (s, 3H); ¹³C{¹H}-NMR (150 MHz, 315 K, CDCl₃) δ [ppm]=163.2, 150.7, 142.8, 139.2, 119.6, 113.8, 102.2, 83.3 (2×), 67.1, 64.5, 45.1, 36.5, 33.9, 27.5, 25.5; HRMS (ESI, 3.5 kV) calc for [M+Na]⁺ C₁₆H₂₀N₂O₅Na⁺ 343.12644, found 343.12604.

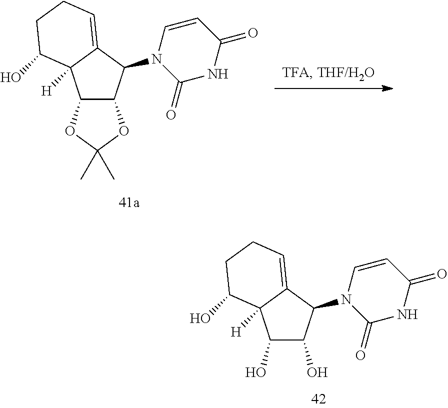 [SMILES: CC1(C)O[C@@H]2[C@H](O1)[C@@H]1C(=CCC[C@H]1O)[C@H]2n1ccc(=O)[nH]c1=O ; "TFA, THF/H2O" ; O=c1ccn([C@@H]2C3=CCC[C@@H](O)[C@@H]3[C@@H](O)[C@H]2O)c(=O)[nH]1]

Alcohol 41a (20.0 mg, 62.4 μmol, 1.00 equiv.) was dissolved in THF (0.5 ml) and TFA (1.0 mL, 10% in H₂O) was added. After 21 h, the solvent was removed in vacuo. Purification by column chromatography (SiO₂, EtOAc 5% MeOH) gave nucleoside analogue 42 (17.0 mg, 60.7 μmol, 97%) as a white solid. R_{f}=0.23 (EtOAc 5% MeOH); [α]_{D}²⁵=−25.1 (c=0.09, MeOH); ¹H-NMR (400 MHz, MeOD) δ [ppm]=7.47 (d, J=8.1 Hz, 1H), 5.70 (d, J=8.0 Hz, 1H), 5.30 (brs, 1H), 5.22 (m, 1H), 4.07 (brs, 1H), 3.97 (brs, 1H), 3.56 (ddd, J=3.8, 9.5, 11.3 Hz, 1H), 2.41 (m, 1H), 2.23 (m, 2H), 1.96 (m, 1H), 1.58 (m, 1H); ¹³C{¹H}-NMR (100 MHz, MeOD) δ [ppm]=166.2, 153.4, 135.4, 121.2, 102.6, 75.5, 73.8, 71.9, 53.5, 32.4, 26.0; HRMS (ESI, 3.5 kV) calc for [M+Na]⁺ C₁₃H₁₆N₂O₅Na⁺ 303.09514, found 303.09520.

### Example 20. Preparation of (1R,2S,3R,3aS,4R)-1-(2,4-dioxo-3,4-dihydropyrimidin-1(2H)-yl)-2,3-dihydroxy-2,3,3a,4,5,6-hexahydro-1H-inden-4-yl isobutyrate (43)

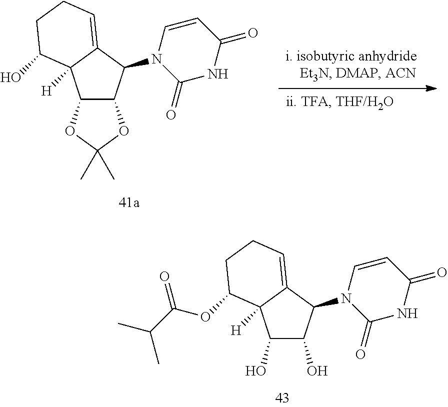

Alcohol 41a (50 mg, 0.16 mmol, 1.00 equiv.) was dissolved in acetonitrile (3 mL) and isobutyric anhydride (52 μL, 0.31 mmol, 2.00 equiv.), Et₃N (44 μL, 0.31 mmol, 2.00 equiv.) and DMAP (1.9 mg, 15.6 μmol, 10 mol %) were added. The reaction was stirred for 4 h, then sat. NaHCO₃ (10 mL) and EtOAc (10 mL) were added. After phase separation, the aqueous phase was extracted with EtOAc (3×15 mL). The combined organic phases were dried over MgSO₄ and concentrated in vacuo. Purification by column chromatography (SiO₂, DCM:EtOAc 1:1) gave the ester (58.0 mg, 0.15 mmol, 97%) as a white solid. For deprotection, an aliquote (15.2 mg, 38.9 μmol) was dissolved in THF (0.5 ml) and TFA (1.0 mL, 10% in H₂O) was added. After 16 h, the solvent was removed in vacuo. Purification by column chromatography (SiO₂, EtOAc 5% MeOH) gave nucleoside analogue 43 (12.6 mg, 35.9 μmol, 92%, 90% over two steps) as a white solid. R_{f}=0.53 (EtOAc 5% MeOH); [α]_{D}²⁵=−44.0 (c=0.11, MeOH); ¹H-NMR (400 MHz, MeOD) δ [ppm]=7.50 (d, J=8.1 Hz, 1H), 5.71 (d, J=8.0 Hz, 1H), 5.32 (m, 1H), 5.27 (brs, 1H), 4.86 (m, 1H), 3.99 (brs, 2H), 2.66 (m, 1H), 2.59 (sep, J=7.0 Hz, 1H), 2.28 (m, 2H), 1.94-2.02 (m, 1H), 1.61-1.73 (m, 1H), 1.20 (d, J=6.9 Hz, 3H), 1.18 (d, J=7.0 Hz, 3H); ¹³C{¹H}-NMR (100 MHz, MeOD) δ [ppm]=178.8, 165.0, 151.8, 144.9, 135.2, 121.5, 102.4, 75.7, 74.0, 73.8, 50.1, 35.4, 29.0, 25.6, 19.4 (2×); HRMS (ESI, 3.5 kV) calc for [M+Na]⁺ C₁₇H₂₂N₂O₆Na⁺ 373.13701, found 373.13701.

### Example 21. Preparation of 1-((1R,2S,3R,3aS,5R)-2,3,5-trihydroxy-2,3,3a,4,5,6-hexahydro-1H-inden-1-yl)pyrimidine-2,4(1H,3H)-dione (44)

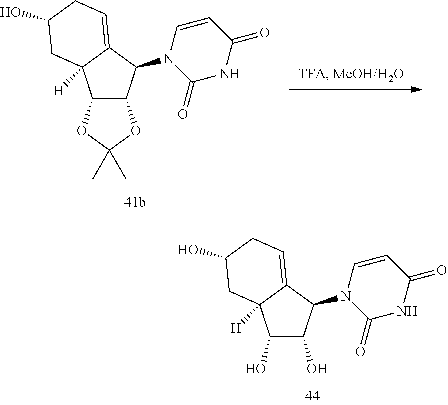 [SMILES: CC1(C)O[C@@H]2[C@H](O1)[C@H]1C[C@H](O)CC=C1[C@H]2n1ccc(=O)[nH]c1=O ; "TFA, MeOH/H2O" ; O=c1ccn([C@@H]2C3=CC[C@@H](O)C[C@@H]3[C@@H](O)[C@H]2O)c(=O)[nH]1]

Alcohol 41b (20.0 mg, 62.4 μmol, 1.00 equiv.) was dissolved in MeOH (0.5 ml) and TFA (0.5 mL, 10% in H₂O) was added. After 16 h, the solvent was removed in vacuo. Purification by column chromatography (SiO₂, EtOAc 5% MeOH) gave nucleoside analogue 44 (16.0 mg, 60.7 μmol, 91%) as a white solid. R_{f}=0.15 (EtOAc 5% MeOH); [α]5=−7.5 (c=0.10, MeOH); ¹H-NMR (600 MHz, MeOD, 315 K) 6 [ppm]=7.43 (d, J=7.9 Hz, 1H), 5.69 (d, J=7.9 Hz, 1H), 5.25 (m, 1H), 5.19 (brs, 1H), 4.17 (s, 1H), 4.05 (s, 1H), 3.84 (s, 1H), 2.71 (s, 1H), 2.30-2.37 (m, 1H), 2.25 (dt, J=4.8, 12.9 Hz, 1H), 2.12 (d, J=18.8 Hz, 1H), 1.40 (ddd, J=2.0, 11.2, 15.1 Hz, 1H); ¹³C{¹H}-NMR (150 MHz, MeOD, 315K) 6 [ppm]=166.3, 152.9, 145.3, 137.6, 119.4, 102.6, 76.9, 76.4, 65.1, 59.3, 39.4, 34.2, 34.0; HRMS (ESI, 3.5 kV) calc for [M+Na]⁺ C₁₃H₁₆N₂O₅Na⁺ 303.09514, found 303.09498.

| | C | H | |
|---|---|---|---|
| # | [ppm] | [ppm] | |
| 2 | 152.9 | — | |
| 4 | 166.3 | — | |
| 5 | 102.6 | 5.69 | |
| 6 | 145.3 | 7.43 | |
| 1′ | 59.3 | 5.19 | |
| 2′ | 76.4 | 4.05 | |
| 3′ | 76.9 | 3.84 | |
| 4′ | 39.4 | 2.71 | |
| 5′ | 34.0 | 2.25 (down) 1.40 (up) | 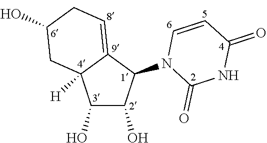 [SMILES: O=c1ccn([C@@H]2C3=CC[C@@H](O)C[C@@H]3[C@@H](O)[C@H]2O)c(=O)[nH]1] |
| 6′ | 65.1 | 4.17 | NOE correlations |
| 7′ | 34.2 | 2.34 (up) 2.12 (down) | 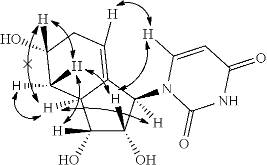 |
| 8′ | 119.4 | 5.25 | |
| 9′ | 137.7 | — | |

### Example 22. Preparation of (1R,2S,3R,3aS,5R)-1-(2,4-dioxo-3,4-dihydropyrimidin-1(2H)-yl)-2,3-dihydroxy-2,3,3a,4,5,6-hexahydro-1H-inden-5-yl isobutyrate (45)

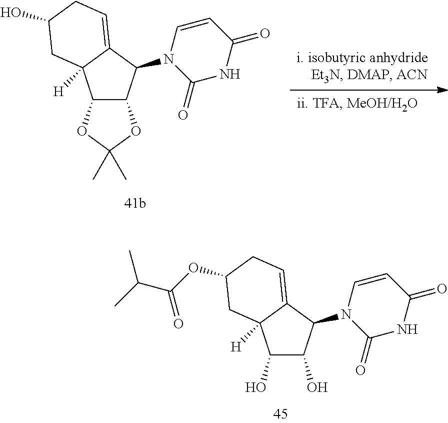

Alcohol 41b (40.0 mg, 0.13 mmol, 1.00 equiv.) was dissolved in acetonitrile (2 mL) and isobutyric anhydride (41 μL, 0.25 mmol, 2.00 equiv.), Et₃N (35 μL, 0.25 mmol, 2.00 equiv.) and DMAP (1.5 mg, 12.5 μmol, 10 mol %) were added. The reaction was stirred for 22 h, then sat. NaHCO₃ (10 mL) and EtOAc (10 mL) were added. After phase separation, the aqueous phase was extracted with EtOAc (3×15 mL). The combined organic phases were dried over MgSO₄ and concentrated in vacuo. Purification by column chromatography (SiO₂, DCM:EtOAc 1:1) gave the ester (27.9 mg, 71.5 μmol, 57%) as a white solid. For deprotection, an aliquote (20.0 mg, 51.2 μmol) was dissolved in MeOH (0.75 ml) and TFA (0.25 mL, 10% in H₂O) was added. After 44 h, the solvent was removed in vacuo. Purification by column chromatography (SiO₂, EtOAc 3% MeOH) gave nucleoside analogue 45 (17.1 mg, 48.8 μmol, 96%, 55% over two steps) as a white solid. nR_{f}=0.23 (EtOAc 3% MeOH); [α]_{D}²⁵=+7.7 (c=0.11, MeOH); ¹H-NMR (400 MHz, MeOD, 295K) 6 [ppm]=7.45 (d, J=7.9 Hz, 1H), 5.69 (d, J=7.9 Hz, 1H), 5.29 (m, 1H), 5.22 (s, 1H), 5.18 (brs, 1H), 4.08 (brs, 1H), 3.86 (brs, 1H), 2.66 (brs, 1H), 2.52 (sep, J=7.0 Hz, 1H), 2.35-2.45 (m, 2H), 2.18 (d, J=19.7 Hz, 1H), 1.47 (ddd, J=2.0, 11.5, 15.2 Hz, 1H), 1.15 (d, J=7.0 Hz, 3H), 1.14 (d, J=7.0 Hz, 3H); ¹³C{¹H}-NMR (150 MHz, CDCl₃, 315 K) δ [ppm]=178.3, 166.5, 153.1, 146.0, 137.9, 118.7, 102.7, 76.9, 76.4, 68.9, 62.9, 39.8, 35.4, 31.4 (2×), 19.3, 19.2; HRMS (ESI, 3.5 kV) calc for [M+Na]⁺ C₁₇H₂₂N₂O₆Na⁺ 373.13701, found 373.13674.

### Example 23. Preparation of 1-((1R,2S,3R,3aS,5S)-2,3,5-trihydroxy-2,3,3a,4,5,6-hexahydro-1H-inden-1-yl)pyrimidine-2,4(1H, 3H)-dione (46)

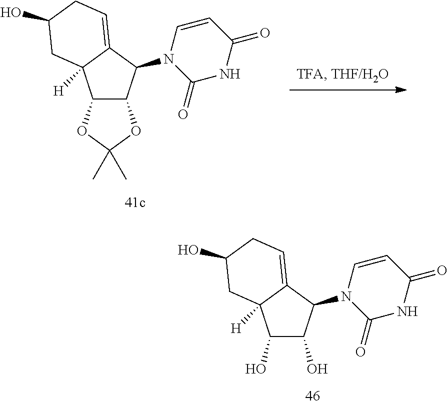 [SMILES: CC1(C)O[C@@H]2[C@H](O1)[C@H]1C[C@@H](O)CC=C1[C@H]2n1ccc(=O)[nH]c1=O ; "TFA, THF/H2O" ; O=c1ccn([C@@H]2C3=CC[C@H](O)C[C@@H]3[C@@H](O)[C@H]2O)c(=O)[nH]1]

Alcohol 41c (24.2 mg, 75.5 μmol, 1.00 equiv.) was dissolved in THF (0.5 ml) and TFA (1 mL, 10% in H₂O) was added. After 44 h, the solvent was removed in vacuo. Purification by column chromatography (SiO₂, EtOAc 7.5% MeOH) gave nucleoside analogue 46 (19.9 mg, 71.0 μmol, 94%) as a white solid. R_{f}=0.15 (EtOAc 5% MeOH); [α]5=−4.6 (c=0.13, MeOH); ¹H-NMR (600 MHz, MeOD, 315 K) 6 [ppm]=7.43 (d, J=7.4 Hz, 1H), 5.69 (d, J=7.8 Hz, 1H), 5.28 (s, 1H), 5.15 (brs, 1H), 4.05 (brs, 1H), 3.90 (m, 1H), 3.82 (brs, 1H), 2.61 (brs, 1H), 2.42 (d, J=17.4 Hz, 1H), 2.32 (m, 1H), 1.93-1.97 (m, 1H), 1.32 (q, J=11.6 Hz, 1H); ¹³C{¹H}-NMR (150 MHz, MeOD, 315 K) 6 [ppm]=166.4, 153.0, 145.5, 137.8, 120.6, 102.6, 76.8, 76.6, 68.2, 62.4, 45.3, 37.2, 35.4; HRMS (ESI, 3.5 kV) calc for [M+Na]⁺ C₁₃H₁₆N₂O₅Na⁺ 303.9514, found 303.09489.

| | C | H | |
|---|---|---|---|
| # | [ppm] | [ppm] | |
| 2 | 153.0 | — | |
| 4 | 166.4 | — | |
| 5 | 102.6 | 5.69 | |
| 6 | 145.5 | 7.43 | |
| 1′ | 62.4 | 5.15 | |
| 2′ | 76.6 | 3.82 | 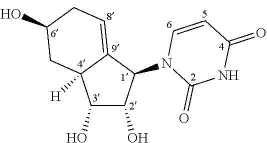 [SMILES: O=c1ccn([C@@H]2C3=CC[C@H](O)C[C@@H]3[C@@H](O)[C@H]2O)c(=O)[nH]1] |
| 3′ | 76.8 | 4.05 | NOE correlations |
| 4′ | 45.3 | 2.61 | 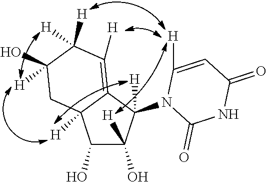 |
| 5′ | 37.2 | 2.32 | |
| | | (down) | |
| | | 1.32 | |
| | | (up) | |
| 6′ | 68.2 | 3.90 | |
| 7′ | 35.4 | 2.42 | |
| | | (down) | |
| | | 1.95 | |
| | | (up) | |
| 8′ | 120.6 | 5.28 | |
| 9′ | 137.8 | — | |

### Example 24. Preparation of (1R,2S,3R,3aS,5S)-1-(2,4-dioxo-3,4-dihydropyrimidin-1(2H)-yl)-2,3-dihydroxy-2,3,3a,4,5,6-hexahydro-1H-inden-5-yl isobutyrate (47)

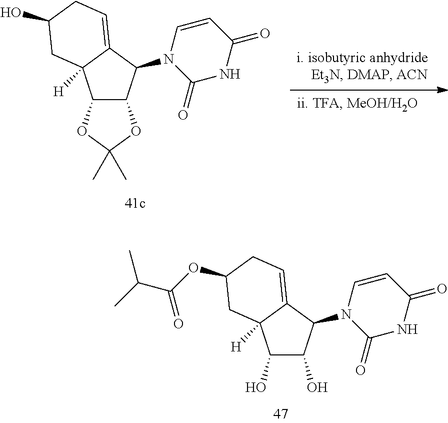

Alcohol 17 (40.0 mg, 0.13 mmol, 1.00 equiv.) was dissolved in acetonitrile (2 mL) and isobutyric anhydride (41 μL, 0.25 mmol, 2.00 equiv.), Et₃N (35 μL, 0.25 mmol, 2.00 equiv.) and DMAP (1.5 mg, 12.5 μmol, 10 mol %) were added. The reaction was stirred for 24 h, then sat. NaHCO₃ (10 mL) and EtOAc (10 mL) were added. After phase separation, the aqueous phase was extracted with EtOAc (3×15 mL). The combined organic phases were dried over MgSO₄ and concentrated in vacuo. Purification by column chromatography (SiO₂, DCM:EtOAc 1:1) gave the ester (45.0 mg, 0.12 mmol, 92%) as a white solid. For deprotection, an aliquote (20.5 mg, 52.5 μmol) was dissolved in MeOH (0.75 ml) and TFA (0.25 mL, 10% in H₂O) was added. After 40 h, the solvent was removed in vacuo. Purification by column chromatography (SiO₂, EtOAc 2% MeOH) gave nucleoside analogue 21b (18.4 mg, 52.5 μmol, quant., 92% over two steps) as a white solid. R_{f}=0.33 (EtOAc 5% MeOH); [α]_{D}²⁵=−11.0 (c=0.13, MeOH); ¹H-NMR (600 MHz, MeOD, 315 K) 6 [ppm]=7.43 (d, J=7.40 Hz, 1H), 5.70 (d, J=7.7 Hz, 1H), 5.31 (s, 1H), 5.12 (brs, 1H), 4.99 (m, 1H), 4.08 (brs, 1H), 3.87 (brs, 1H), 2.68 (brs, 1H), 2.54 (sep, J=6.9 Hz, 1H), 2.50 (m, 1H), 2.33-2.38 (m, 1H), 2.05-2.12 (m, 1H), 1.46 (q, J=11.6 Hz, 1H), 1.15 (d, J=6.9 Hz, 3H), 1.14 (d, J=6.9 Hz, 3H); ¹³C{¹H}-NMR (100 MHz, CDCl₃) δ [ppm]=178.4, 166.3, 152.7, 145.3, 138.0, 119.8, 102.7, 76.8, 76.6, 71.2, 44.7, 35.3, 33.5, 31.8, 19.2 (2×); HRMS (ESI, 3.5 kV) calc for [M+Na]⁺ C₁₇H₂₂N₂O₆Na⁺ 373.13701, found 373.13688.

### Example 25. Preparation of 4-(hydroxyimino)-1-((1R,2S,3R,3aR,4R)-2,3,4-trihydroxy-2,3,3a,4,5,6-hexahydro-1H-inden-1-yl)-3,4-dihydropyrimidin-2(1H)-one (48)

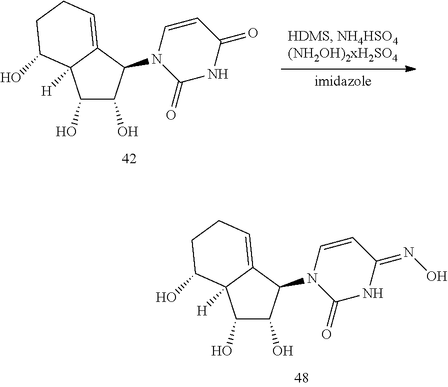 [SMILES: O=c1ccn([C@@H]2C3=CCC[C@@H](O)[C@@H]3[C@@H](O)[C@H]2O)c(=O)[nH]1 ; "HDMS, NH4HSO4(NH2OH)2xH2SO4" ; "imidazole" ; O=c1[nH]/c(=N\O)ccn1[C@@H]1C2=CCC[C@@H](O)[C@@H]2[C@@H](O)[C@H]1O]

Uracil analogue 42 (1.00 eq., 0.13 mmol, 35 mg) was dissolved in HMDS (1.5 mL) and NH₄HSO₄ (4.00 eq., 0.50 mmol, 57 mg), (NH₂OH)xH₂SO₄ (2.00 eq., 0.25 mmol, 41 mg) and imidazole (0.50 eq., 62.4 μmol, 4.3 mg) were added. The solution was stirred at 85° C. for 24 h. Then the reaction was quenched with water (3 mL) and the slurry was extracted with hexane (2×5 mL). The combined organic phases were dried over MgSO4. The solvent was removed in vacuo and the residue was dissolved in MeOH (1 mL) and TFA (50 μL) was added. After 15 min, the volatiles were removed in vacuo. Purification by column chromatography (SiO₂, EtOAc 5% MeOH) gave nucleoside analogue 48 (25.7 mg, 87.0 μmol, 69%) as a white solid. R_{f}=0.25 (EtOAc 5% MeOH); 1H-NMR (400 MHz, MeOD) δ [ppm]=6.88 (d, J=8.20 Hz, 1H), 5.66 (d, J=8.0 Hz, 1H), 5.28 (s, 1H), 4.03 (brs, 1H), 3.92 (brs, 1H), 3.55 (m, 1H), 2.39 (brs, 1H), 2.23 (m, 2H), 1.96 (m, 1H), 1.58 (m, 1H). HRMS (ESI, 3.5 kV) calc for [M+Na]⁺ C₁₃H₁₇N₃O₅Na⁺ 318.10604, found 318.10579.

### Example 26. Preparation of (1R,2S,3R,3aS,4R)-2,3-dihydroxy-1-(4-(hydroxyimino)-2-oxo-3,4-dihydropyrimidin-1(2H)-yl)-2,3,3a,4,5,6-hexahydro-1H-inden-4-yl isobutyrate (49)

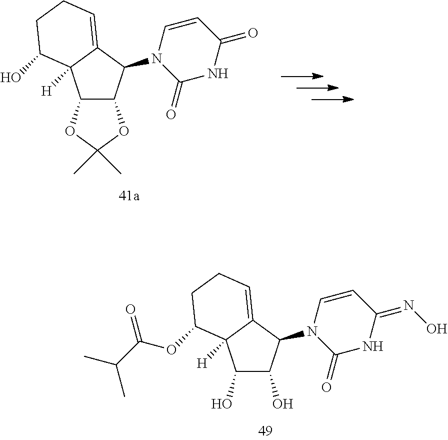 [SMILES: CC1(C)O[C@@H]2[C@H](O1)[C@@H]1C(=CCC[C@H]1O)[C@H]2n1ccc(=O)[nH]c1=O ; CC(C)C(=O)O[C@@H]1CCC=C2[C@H]1[C@@H](O)[C@@H](O)[C@@H]2n1cc/c(=N/O)[nH]c1=O]

Analogue 49 was synthesized using the methods outlined in example 20 and 25 using intermediate 41a. R_{f}=0.34 (EtOAc 5% MeOH); ¹H-NMR (400 MHz, MeOD) δ [ppm]=7.10 (d, J=7.6 Hz, 1H), 5.74 (d, J=8.0 Hz, 1H), 5.36 (s, 1H), 4.89 (m, 1H), 3.94 (m, 2H), 2.64 (brs, 1H), 2.58 (m, 1H), 2.26 (brs, 2H), 1.95 (m, 1H), 1.66 (m, 1H), 1.18 (d, J=6.7 Hz, 3H), 1.16 (d, J=6.7 Hz, 3H). [M+Na]⁺ C₁₇H₂₃N₃O₆Na⁺ 388.14791, found 388.14748.

### Example 27. Preparation of 4-(hydroxyimino)-1-((1R,2S,3R,3aS,5R)-2,3,5-trihydroxy-2,3,3a,4,5,6-hexahydro-1H-inden-1-yl)-3,4-dihydropyrimidin-2(1H)-one (50)

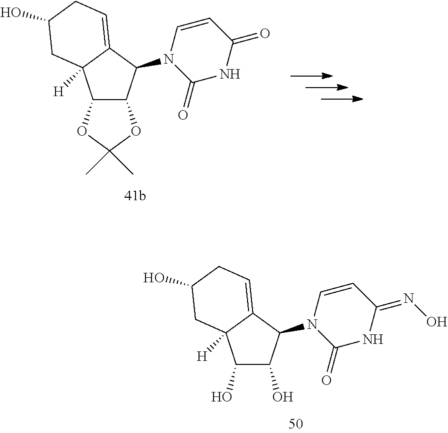 [SMILES: CC1(C)O[C@@H]2[C@H](O1)[C@H]1C[C@H](O)CC=C1[C@H]2n1ccc(=O)[nH]c1=O ; O=c1[nH]/c(=N\O)ccn1[C@@H]1C2=CC[C@@H](O)C[C@@H]2[C@@H](O)[C@H]1O]

Analogue 50 was synthesized using the methods outlined in example 21 and 25 using intermediate 41b. R_{f}=0.22 (EtOAc 5% MeOH); ¹H-NMR (400 MHz, MeOD) δ [ppm]=6.79 (brs, 1H), 5.62 (d, J=8.2 Hz, 1H), 5.29 (brs, 1H), 4.17 (brs, 1H), 4.00 (brs, 1H), 3.78 (m, 1H), 2.67 (brs, 1H), 2.19-2.40 (m, 2H), 2.11 (d, J=18.8 Hz, 1H), 1.38 (ddd, J=1.9, 11.2, 12.7 Hz, 1H). [M+Na]⁺ C₁₃H₁₇N₃O₅Na⁺ 318.10604, found 318.10593.

### Example 28. Preparation of (1R,2S,3R,3aS,5R)-2,3-dihydroxy-1-(4-(hydroxyimino)-2-oxo-3,4-dihydropyrimidin-1(2H)-yl)-2,3,3a,4,5,6-hexahydro-1H-inden-5-yl isobutyrate

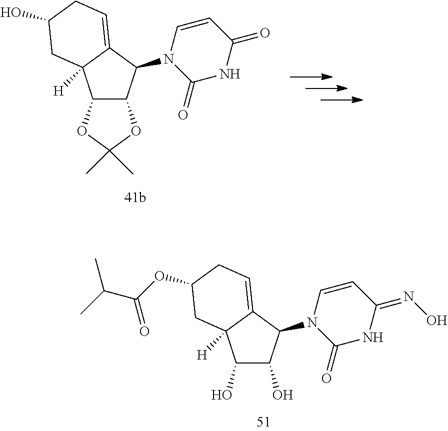 [SMILES: CC1(C)O[C@@H]2[C@H](O1)[C@H]1C[C@H](O)CC=C1[C@H]2n1ccc(=O)[nH]c1=O ; CC(C)C(=O)O[C@@H]1CC=C2[C@@H](n3cc/c(=N/O)[nH]c3=O)[C@H](O)[C@H](O)[C@H]2C1]

Analogue 51 was synthesized using the methods outlined in example 22 and 25 using intermediate 41b. R_{f}=0.30 (EtOAc 5% MeOH); ¹H-NMR (400 MHz, MeOD) δ [ppm]=6.72 (brs, 1H), 5.59 (d, J=7.9 Hz, 1H), 5.32 (brs, 1H), 5.21 (brs, 1H), 4.01 (brs, 1H), 3.79 (brs, 1H), 2.62 (brs, 1H), 2.53 (hep, J=7.1 Hz, 1H), 2.32-2.47 (m, 2H), 2.17 (d, J=19.9 Hz, 1H), 1.44 (ddd, J=1.9, 11.8, 13.3 Hz, 1H), 1.15 (d, J=7.0 Hz, 3H), 1.13 (d, J=7.0 Hz, 3H). [M+Na]⁺ C₁₇H₂₃N₃O₆Na⁺ 388.14791, found 388.14730.

### Example 29. Preparation of 4-(hydroxyimino)-1-((1R,2S,3R,3aS,5S)-2,3,5-trihydroxy-2,3,3a,4,5,6-hexahydro-1H-inden-1-yl)-3,4-dihydropyrimidin-2(1H)-one (52)

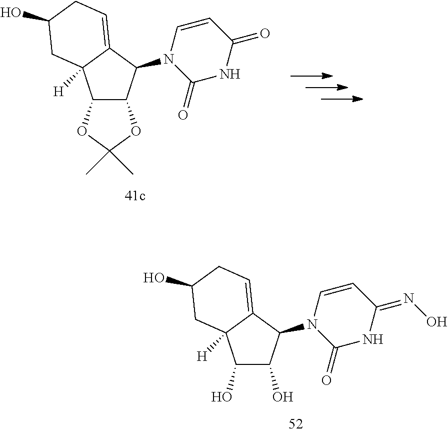 [SMILES: CC1(C)O[C@@H]2[C@H](O1)[C@H]1C[C@@H](O)CC=C1[C@H]2n1ccc(=O)[nH]c1=O ; O=c1[nH]/c(=N\O)ccn1[C@@H]1C2=CC[C@H](O)C[C@@H]2[C@@H](O)[C@H]1O]

Analogue 52 was synthesized using the methods outlined in example 23 and 25 using intermediate 41c. R_{f}=0.25 (EtOAc 5% MeOH); ¹H-NMR (400 MHz, MeOD) δ [ppm]=6.86 (brs, 1H), 5.68 (d, J=8.1 Hz, 1H), 5.32 (brs, 1H), 4.01 (brs, 1H), 3.90 (brs, 1H), 3.78 (m, 1H), 2.59 (brs, 1H), 2.42 (m, 1H), 2.31 (m, 1H), 1.95 (m, 1H), 1.30 (q, J=11.3 Hz, 1H). [M+Na]+C₁₃H₁₇N₃O₅Na⁺ 318.10604, found 318.10587.

### Example 30. Preparation of (1R,2S,3R,3aS,5S)-2,3-dihydroxy-1-(4-(hydroxyimino)-2-oxo-3,4-dihydropyrimidin-1(2H)-yl)-2,3,3a,4,5,6-hexahydro-1H-inden-5-yl isobutyrate (53)

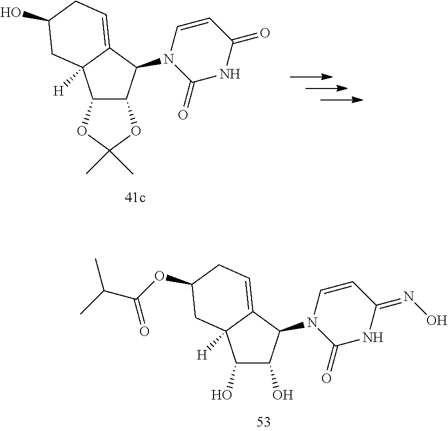 [SMILES: CC1(C)O[C@@H]2[C@H](O1)[C@H]1C[C@@H](O)CC=C1[C@H]2n1ccc(=O)[nH]c1=O ; CC(C)C(=O)O[C@H]1CC=C2[C@@H](n3cc/c(=N/O)[nH]c3=O)[C@H](O)[C@H](O)[C@H]2C1]

Analogue 53 was synthesized using the methods outlined in example 24 and 25 using intermediate 41c. R_{f}=0.27 (EtOAc 5% MeOH).

### Example 31. Preparation of 4-amino-1-((1R,2S,3R,3aR,4R)-2,3,4-trihydroxy-2,3,3a,4,5,6-hexahydro-1H-inden-1-yl)pyrimidin-2(1H)-one (55)

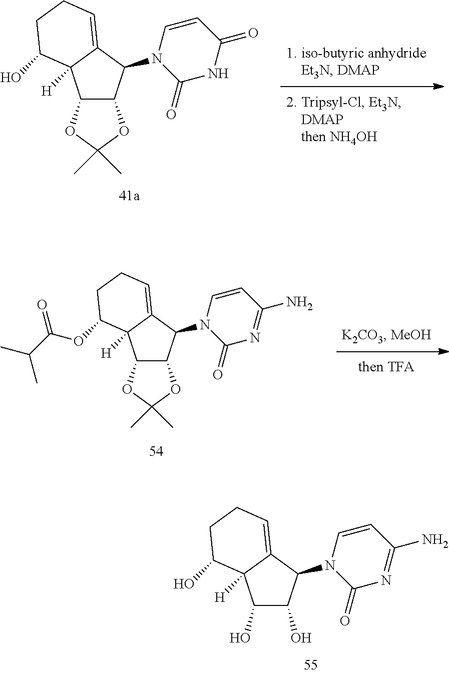 [SMILES: CC1(C)O[C@@H]2[C@H](O1)[C@@H]1C(=CCC[C@H]1O)[C@H]2n1ccc(=O)[nH]c1=O ; CC(C)C(=O)O[C@@H]1CCC=C2[C@H]1[C@H]1OC(C)(C)O[C@H]1[C@@H]2n1ccc(N)nc1=O ; Nc1ccn([C@@H]2C3=CCC[C@@H](O)[C@@H]3[C@@H](O)[C@H]2O)c(=O)n1]

Intermediate 41a (1.00 eq., 1.00 mmol) is dissolved in ACN (10 mL) and Et₃N (2.50 eq.), iso-butyric anhydride (2.50 eq.) and DMAP (0.10 eq.) are added. The reaction is stirred until completion. Workup and purification yielding in iso-butyric ester. The intermediate (1.00 eq., 1.00 mmol) is dissolved in ACN (10 mL) and tripsyl chloride (2.00 eq.), Et₃N (2.00 eq) and DMAP (0.1 eq.) are added. After completion of the reaction NH₄OH (25% in water) is added. After 2 h workup and purification yielding in protected cytidine type analogue 54. For deprotection, 54 (1.00 eq., 1.00 mmol) is dissolved in MeOH (5 mL) and K₂CO₃ (1.50 eq.) is added. After complete saponification, TFA (20% in H₂O, 5 mL) is added. The reaction is stirred until completion. Workup and purification yielding in final cytidine analogue 55.

### Example 32. Preparation of 4-amino-1-((1R,2S,3R,3aS,5R)-2,3,5-trihydroxy-2,3,3a,4,5,6-hexahydro-1H-inden-1-yl)pyrimidin-2(1H)-one (56)

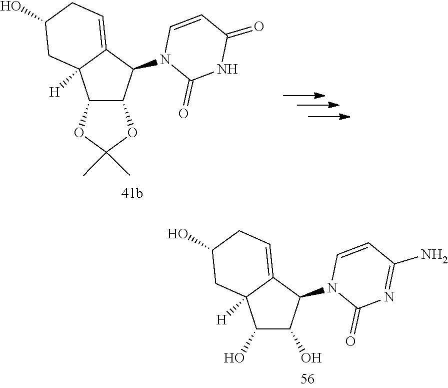 [SMILES: CC1(C)O[C@@H]2[C@H](O1)[C@H]1C[C@H](O)CC=C1[C@H]2n1ccc(=O)[nH]c1=O ; Nc1ccn([C@@H]2C3=CC[C@@H](O)C[C@@H]3[C@@H](O)[C@H]2O)c(=O)n1]

Analogue 56 is synthesized using the methods outlined in example 31 using intermediate 41b.

### Example 33. Preparation of 4-amino-1-((1R,2S,3R,3aS,5S)-2,3,5-trihydroxy-2,3,3a,4,5,6-hexahydro-1H-inden-1-yl)pyrimidin-2(1H)-one (57)

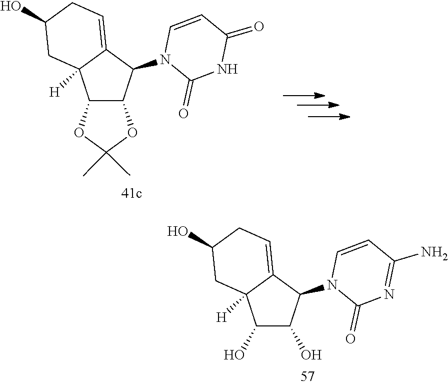 [SMILES: CC1(C)O[C@@H]2[C@H](O1)[C@H]1C[C@@H](O)CC=C1[C@H]2n1ccc(=O)[nH]c1=O ; Nc1ccn([C@@H]2C3=CC[C@H](O)C[C@@H]3[C@@H](O)[C@H]2O)c(=O)n1]

Analogue 57 is synthesized using the methods outlined in example 31 using intermediate 41c.

### Example 34. Preparation of 5-fluoro-1-((1R,2S,3R,3aR,4R)-2,3,4-trihydroxy-2,3,3a,4,5,6-hexahydro-1H-inden-1-yl)pyrimidine-2,4(1H,3H)-dione (59)

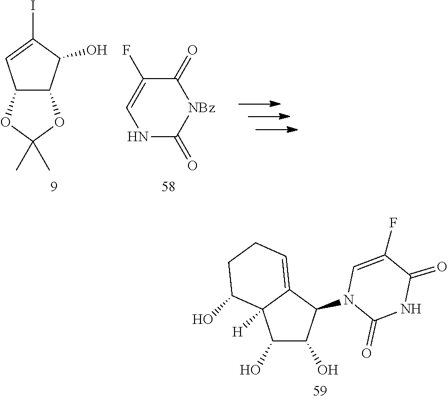

Analogue 59 is synthesized using the methods outlined in example 19 using intermediates 9 and 58.

### Example 35. Preparation of 5-fluoro-1-((1R,2S,3R,3aS,5R)-2,3,5-trihydroxy-2,3,3a,4,5,6-hexahydro-1H-inden-1-yl)pyrimidine-2,4(1H,3H)-dione (60)

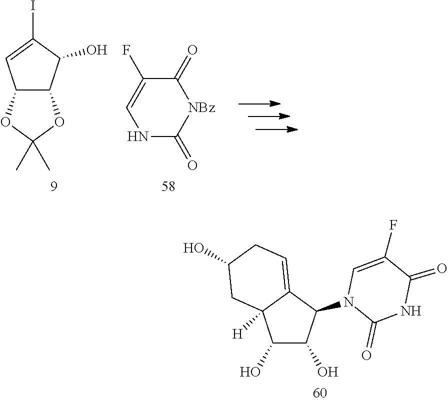

Analogue 60 is synthesized using the methods outlined in example 19 and 21 using intermediates 9 and 58.

### Example 36. Preparation of 5-fluoro-1-((1R,2S,3R,3aS,5S)-2,3,5-trihydroxy-2,3,3a,4,5,6-hexahydro-1H-inden-1-yl)pyrimidine-2,4(1H,3H)-dione (61)

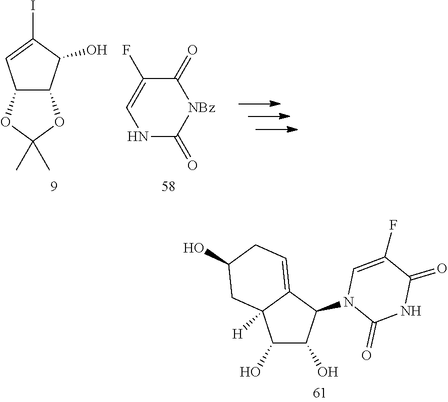

Analogue 61 is synthesized using the methods outlined in example 19 and 23 using intermediates 9 and 58.

### Example 37. Preparation of (1R,2S,3R,7R,7aR)-3-(4-amino-7H-pyrrolo[2,3-d]pyrimidin-7-yl)-2,3,5,6,7,7a-hexahydro-1H-indene-1,2,7-triol (63)

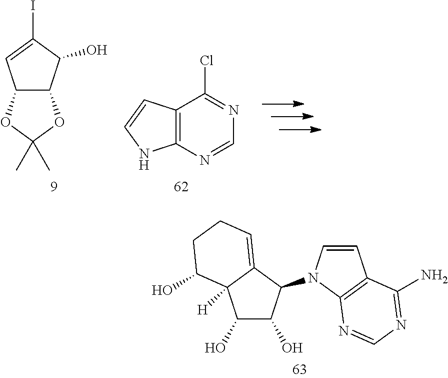 [SMILES: CC1(C)O[C@@H]2[C@@H](C=C(I)[C@@H]2O)O1 ; Clc1ncnc2[nH]ccc12 ; Nc1ncnc2c1ccn2[C@@H]1C2=CCC[C@@H](O)[C@@H]2[C@@H](O)[C@H]1O]

Analogue 63 was synthesized using the methods outlined in example 2 using intermediates 9 and 62. ¹H-NMR (600 MHz, MeOD) δ [ppm]=8.07 (s, 1H), 7.09 (d, J=3.8 Hz, 1H), 6.64 (d, J=3.9 Hz, 1H), 5.48 (brs, 1H), 4.90 (m, 1H), 4.15 (m, 2H), 3.61 (m, 1H), 2.52 (brs, 1H), 2.15 (m, 2H), 1.96 (m, 1H), 1.59 (m, 1H).

### Example 38. Preparation of (1R,2S,3R,6S,7aS)-3-(4-amino-7H-pyrrolo[2,3-d]pyrimidin-7-yl)-2,3,5,6,7,7a-hexahydro-1H-indene-1,2,6-triol (64)

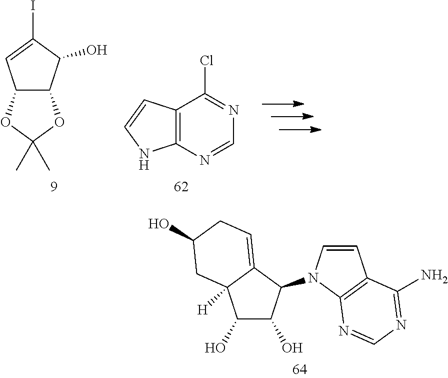 [SMILES: CC1(C)O[C@@H]2[C@@H](C=C(I)[C@@H]2O)O1 ; Clc1ncnc2[nH]ccc12 ; Nc1ncnc2c1ccn2[C@@H]1C2=CC[C@H](O)C[C@@H]2[C@@H](O)[C@H]1O]

Analogue 64 was synthesized using the methods outlined in example 2 and 4 using intermediates 9 and 62. ¹H-NMR (400 MHz, MeOD) δ [ppm]=8.07 (s, 1H), 7.02 (d, J=3.9 Hz, 1H), 6.62 (d, J=3.9 Hz, 1H), 5.43 (brs, 1H), 5.01 (m, 1H), 4.10 (m, 1H), 3.91 (m, 2H), 2.72 (brs, 1H), 2.36 (m, 2H), 1.88 (m, 1H), 1.36 (q, J=12.0 Hz, 1H).

### Example 39. Preparation of (1S,3S,7R,7aR)-3-(6-amino-9H-purin-9-yl)-2,3,5,6,7,7a-hexahydro-1H-indene-1,7-diol (67)

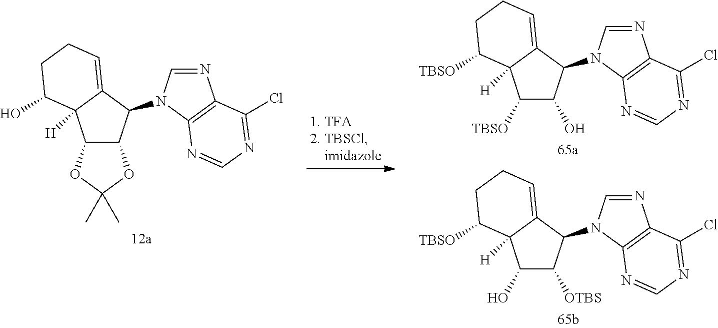

Protected diol 12a (1.00 mmol) is dissolved in MeOH (5 mL) and TFA (20% in H₂O, 5 mL) is added. After 1d the volatiles were removed in vacuo. Then the crude was dissolved in DMF (5 mL) and imidazole (2.50 eq.) and TBSCl (2.50 eq.) is added subsequently. The reaction is stirred for 5 h, then workup and purification yield 65a and 65b.

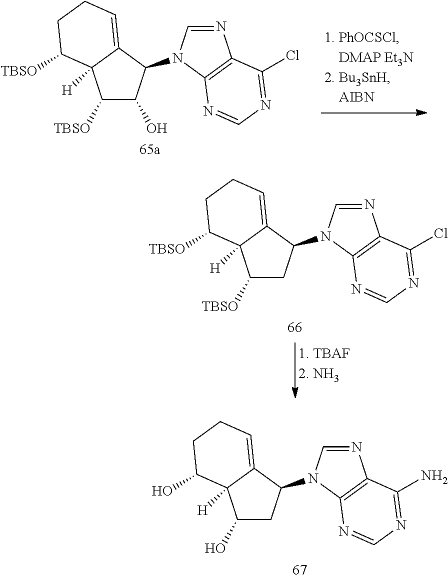 [SMILES: CC(C)(C)[Si](C)(C)O[C@H]1[C@@H](O)[C@H](n2cnc3c(Cl)ncnc32)C2=CCC[C@@H](O[Si](C)(C)C(C)(C)C)[C@@H]21 ; CC(C)(C)[Si](C)(C)O[C@H]1C[C@H](n2cnc3c(Cl)ncnc32)C2=CCC[C@@H](O[Si](C)(C)C(C)(C)C)[C@@H]21 ; Nc1ncnc2c1ncn2[C@H]1C[C@H](O)[C@@H]2C1=CCC[C@H]2O]

Alcohol 65a (1.00 eq., 1.00 mmol) is dissolved in DCM (10 mL) and PhOCSCl (3.00 eq., 3.00 mmol), DMAP (0.10 eq., 0.10 mmol) and Et₃N (3.00 eq., 3.00 mmol) are added. The reaction is stirred until completion then workup and purification yield the thionocarbonate intermediate. This is dissolved in toluene (10 mL) and Bu₃SNH (4.00 eq.) and AIBN (0.10 eq.) are added. The reaction is stirred until completion under reflux, then workup and purification yield 66. Finally, 66 is dissolved in THF (10 mL) and TBAF (1M in THF, 1.00 eq.) is added. After complete deprotection workup yield the crude product which is dissolved in ACN (5 mL) and NH₄OH solution (25% in H2O, 3 mL) is added. The reaction is stirred at 85° C. until completion. Workup and purification yield the desired analogue 67.

### Example 40. Preparation of (1R,2S,3R,3aR,4R)-1-(6-amino-9H-purin-9-yl)-3-methoxy-2,3,3a,4,5,6-hexahydro-1H-indene-2,4-diol (69)

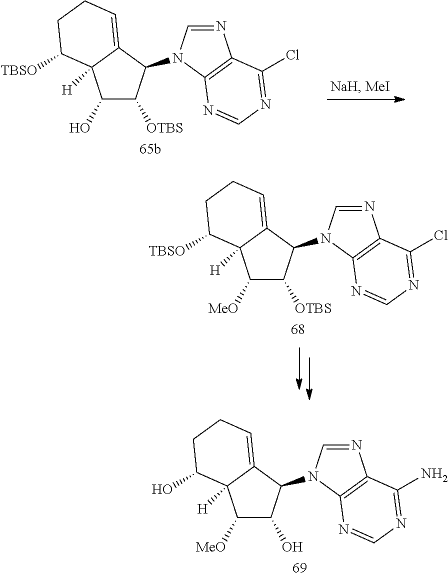

Alcohol 65b (1.00 eq., 1.00 mmol) is dissolved in THF (10 mL) and at 0° C. NaH (1.20 eq., 60%) and Mel (5.00 eq.) are added. The reaction is stirred until completion and workup and purification yield 68. Protected 68 is transformed into 69 with the methods described in example 39.

### Example 41. Preparation of 1-((1S,3S,3aS,5S)-3,5-dihydroxy-2,3,3a,4,5,6-hexahydro-1H-inden-1-yl)-5-methylpyrimidine-2,4(1H,3H)-dione (71)

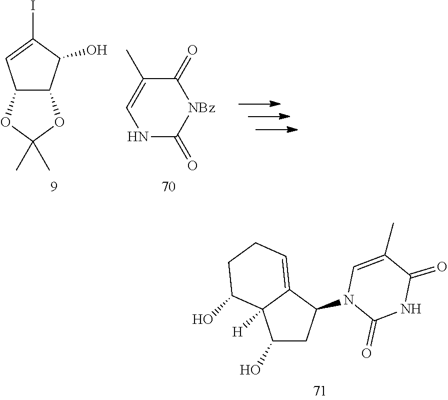

Analogue 71 is synthesized using the methods outlined in example 19 and 39 using intermediates 9 and 70.

### Example 42. Preparation of 2-amino-9-((1R,2S,3R,3aR,4R)-2,3,4-trihydroxy-2,3,3a,4,5,6-hexahydro-1H-inden-1-yl)-1,9-dihydro-6H-purin-6-one

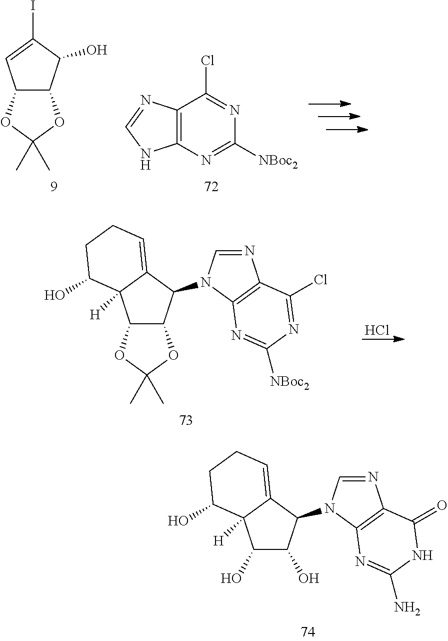

Analogue 73 is synthesized using the methods outlined in example 2 using intermediates 9 and 72. Protected 73 (1.00 eq., 1.00 mmol) is then dissolved in MeOH (10 mL) and HCl (2M in H₂O, 10 mL) is added. After completion of the reaction workup and purification yield 74.

### Example 43. Antiviral Activity and Cell Viability

Antiviral data were obtained using HEp-2 cells and RSV-A Long strain (HRSVA/Maryland USA/Long/1956, sequence ID: OK649668.1], 99% identical in positions 1716-2119 and 100% identical in positions 5186-5590).

For antiviral assessment HEp-2 cells were seeded at 5×10⁴ cells per well in 96-well plates the day before infection. All compounds were dissolved in dimethyl sulfoxide at 8 mM and further dilute serially in DMEM containing 0.5% DMSO to final concentrations. The supernatant of the cultures were collected and frozen at −80° C. at 48 hours post-infection for cell viability analysis and viral titration.

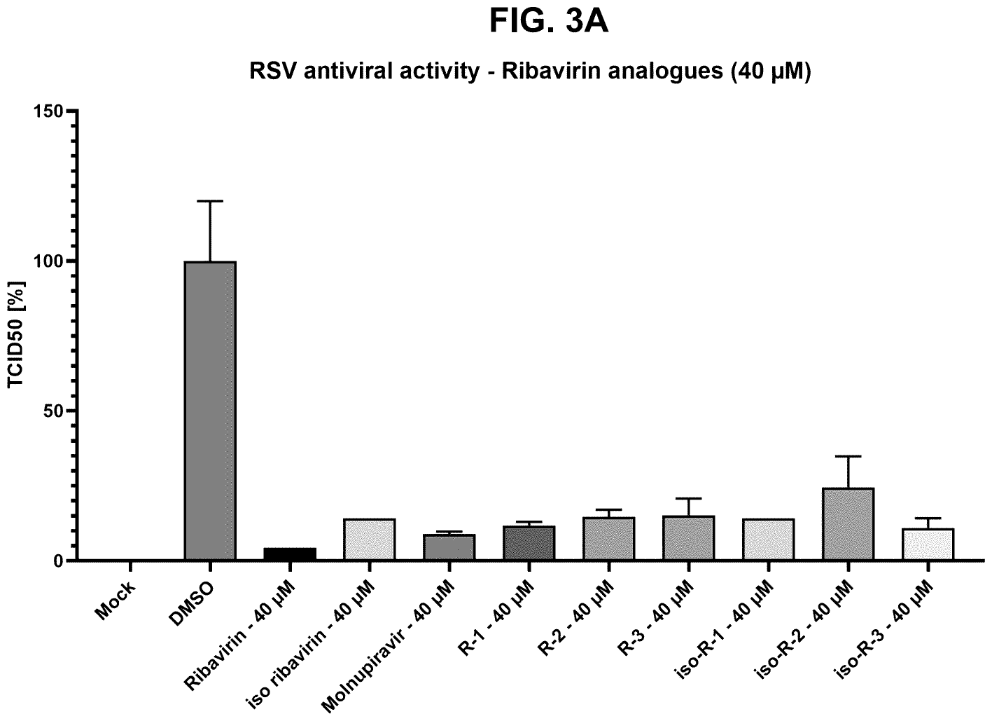

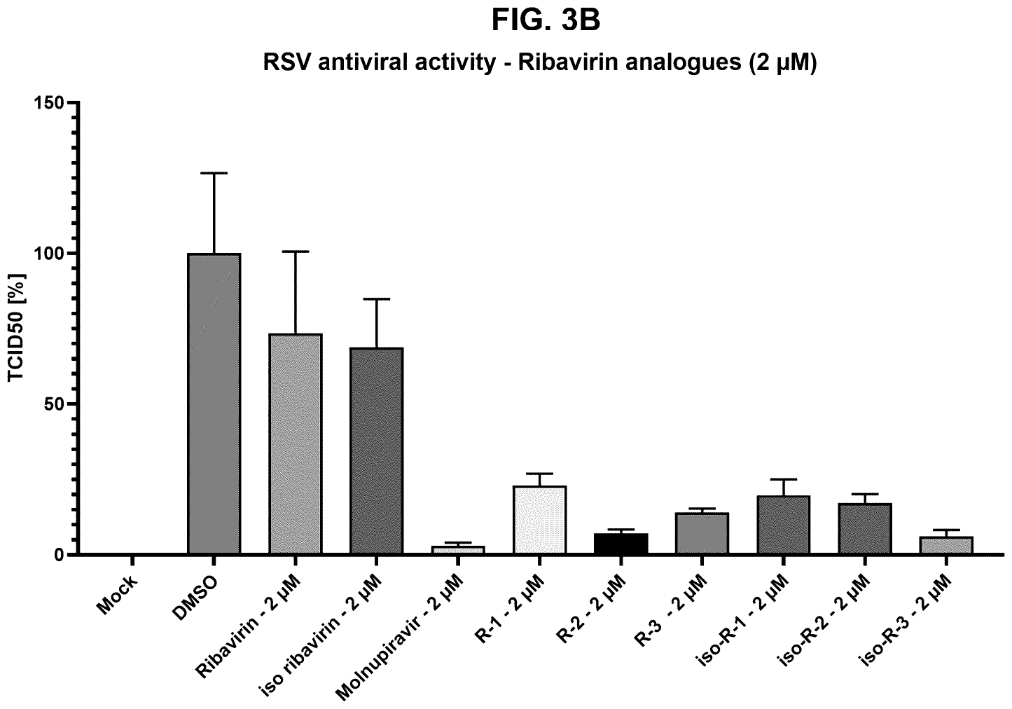

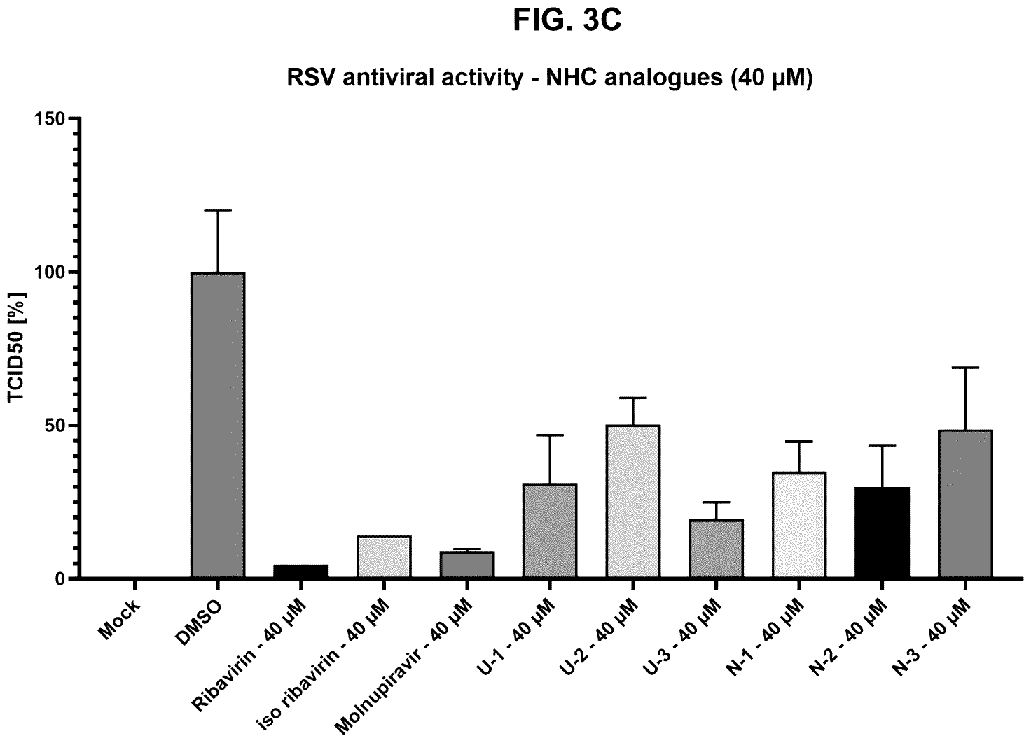

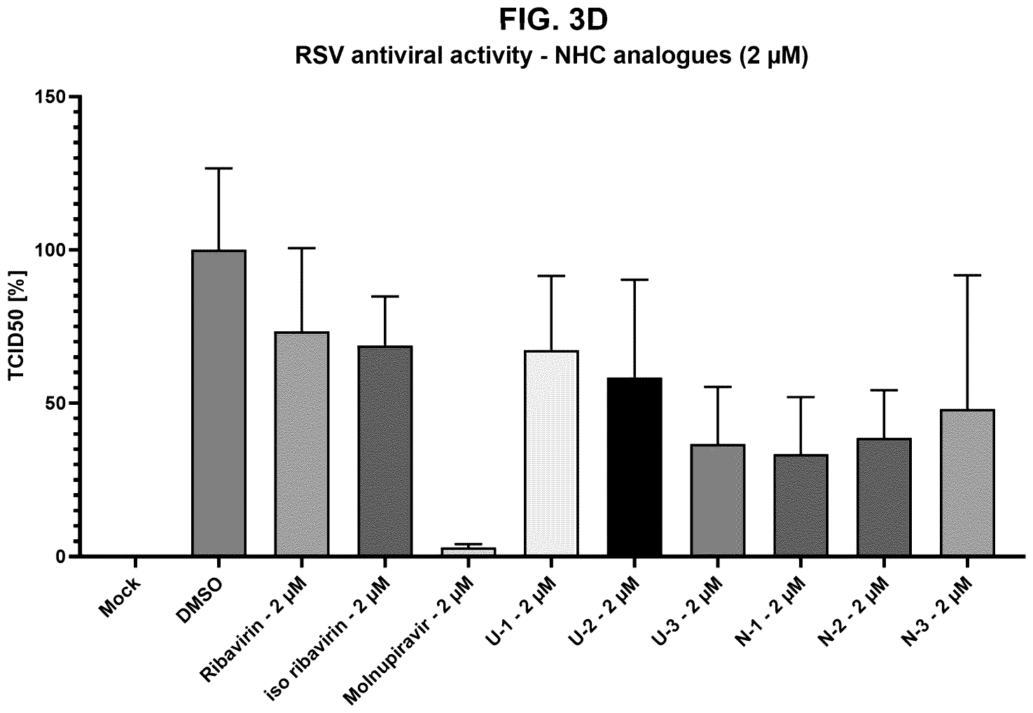

HEp-2 cells were seeded on 96-well tissue culture plates one day before the viral titration assay. Virus samples or culture supernatants were titrated in serial half-log 10 dilutions with the corresponding culture medium before adding the diluted virus to the cell plates in quadruplicate. The highest viral dilution leading to CPE was recorded and the 50% tissue culture infectious dose (TCID50) was calculated using the Spearman-Karber method. The results are shown in FIGS. 3A-3D.

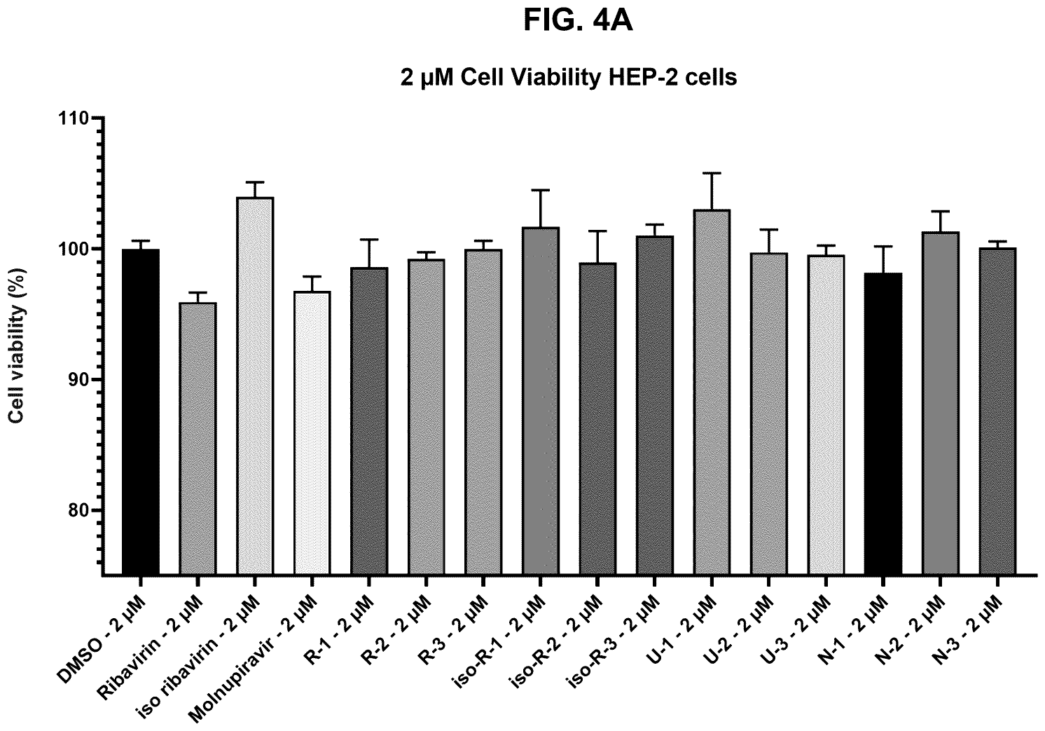

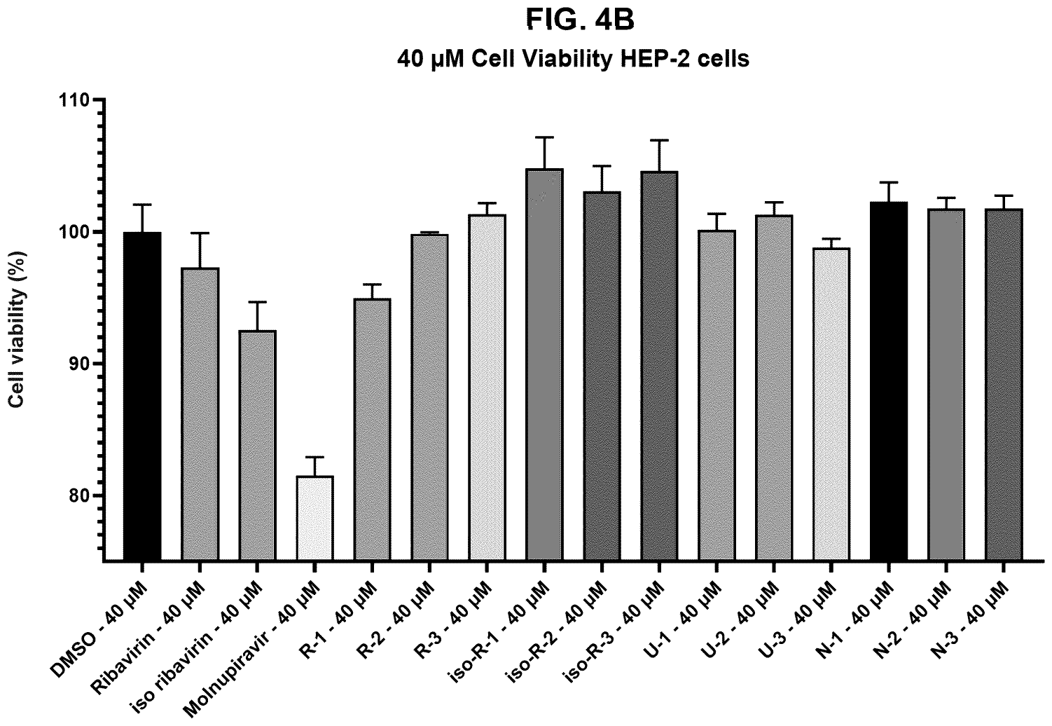

In addition, cytotoxicity assays were performed with thiazolyl blue tetrazolium bromide (MTT, Thermo Fisher, Carlsbad, CA, M6494) cell viability assay after incubation with the compounds for 48 hours. The results are shown in FIGS. 4A and 4B.

The compounds used in FIGS. 3A-3D, 4A, and 4B are shown below:

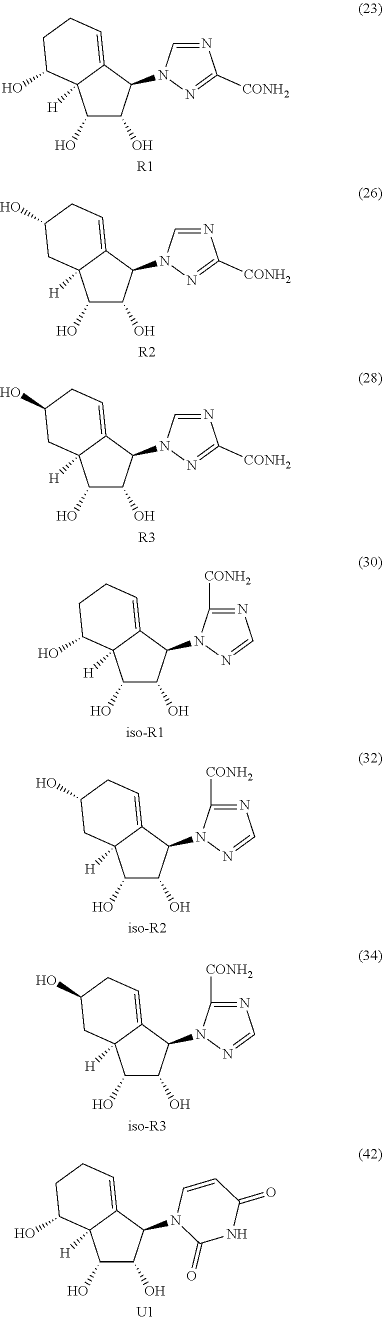

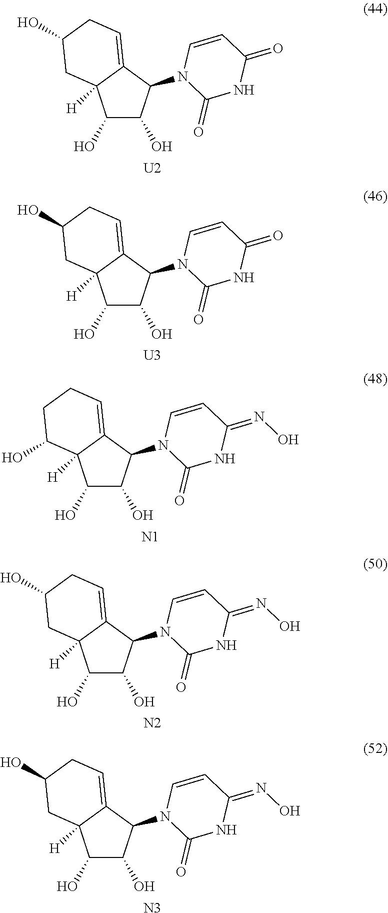

Although the foregoing has been described in some detail by way of illustration and example for purposes of clarity and understanding, one of skill in the art will appreciate that certain changes and modifications can be practiced within the scope of the appended claims.

In addition, each reference provided herein is incorporated by reference in its entirety to the same extent as if each reference was individually incorporated by reference.

1. A compound according to the following formula: 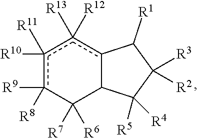
a pharmaceutically acceptable salt thereof, or an enantiomer thereof, wherein:
R¹ is nitrogen-containing monocyclic or bicyclic heterocyclyl;
each of R², R³, R⁴, R⁵, R⁶, and R⁷ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, optionally substituted carboxylate, or OPₓ;
each of R⁸, R⁹, R¹⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted heteroaryloxy, optionally substituted carboxylate, OPₓ, or absent;
Pₓ is monophosphate, diphosphate, triphosphate, C2-6 acyl, or a phosphate prodrug moiety; and
each dashed line represents a single or double bond, provided that no more than one double bond is present.

2. The compound of claim 1, or a pharmaceutically acceptable salt thereof, comprising a structure according to the following formula:

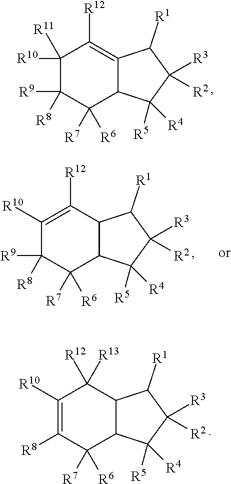

3. The compound of claim 1, or a pharmaceutically acceptable salt thereof, comprising a structure according to the following formula:

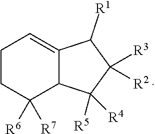

4. The compound of claim 3, or a pharmaceutically acceptable salt thereof, comprising a structure according to the following formula:

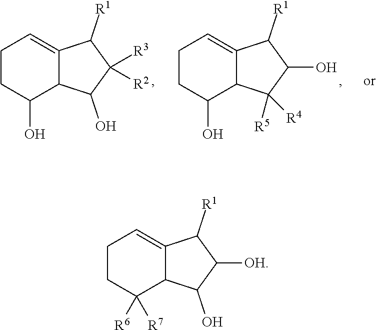

5. (canceled)

6. (canceled)

7. The compound of claim 1, or a pharmaceutically acceptable salt thereof, comprising a structure according to the following formula:

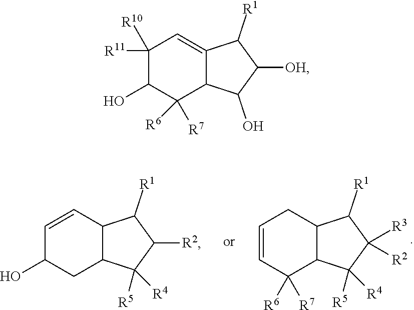

8. (canceled)

9. (canceled)

10. The compound of claim 9, or a pharmaceutically acceptable salt thereof, comprising a structure according to the following formula:

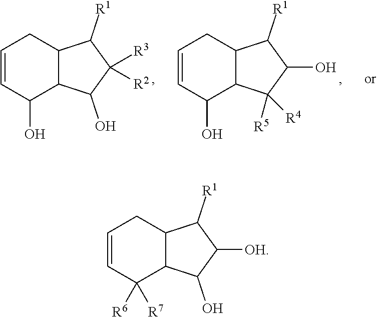

11. (canceled)

12. The compound of claim 1, wherein each of R², R³, R⁴, and R⁵ is, independently, H, deuterium, hydroxyl, Cl, F, CN, Me, Et, (C₁₋₆)OH, ORₓ, (C₁₋₆)COORₓ, or (C₁₋₆)CON(Rₓ)₂;
each of R⁶ and R⁷ is, independently, H, deuterium, hydroxyl, Cl, F, CN, Me, Et, (C₁₋₆)OH, ORₓ, (C₁₋₆)COORₓ, (C₁₋₆)CON(Rₓ)₂, optionally substituted carboxylate, or OPₓ;
Pₓ is monophosphate, diphosphate, triphosphate, C₂₋₆ acyl, or a phosphate prodrug moiety; and
each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted carboxylate, or optionally substituted heteroaryloxy.

13. The compound of claim 1, or a pharmaceutically acceptable salt thereof, comprising a structure according to the following formula: 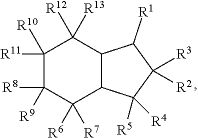
wherein:
R¹ is optionally substituted nitrogen-containing monocyclic or bicyclic heterocyclyl;
each of R², R³, R⁴, R⁵, R⁶, R⁷, R⁹, R⁹, R¹⁰, R¹¹, R¹², and R¹³ is, independently, H, deuterium, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy, optionally substituted carboxylate, or OPₓ,
wherein Pₓ is monophosphate, diphosphate, triphosphate, C₂₋₆ acyl, or a phosphate prodrug moiety.

14. The compound of claim 13, or a pharmaceutically acceptable salt thereof, comprising a structure according to the following formula: 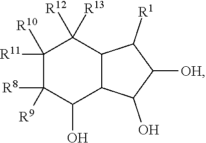
wherein:
R¹ is optionally substituted nitrogen-containing monocyclic or bicyclic heterocyclyl;
each of R¹⁰, R¹¹, R¹², and R¹³ is, independently, H, deuterium, hydroxyl, Cl, F, CN, Me, Et, (C₁₋₆)OH, ORₓ, (C₁₋₆)COORₓ, (C₁₋₆)CON(Rₓ)₂, epoxide, or cyclopropane, each of R¹, R⁹ is, independently, H, deuterium, hydroxyl, C₁, F, CN, Me, Et, (C₁₋₆)OH, ORₓ, (C₁₋₆)COORₓ, (C₁₋₆)CON(Rₓ)₂, epoxide, cyclopropane, or OPₓ,
Pₓ is monophosphate, diphosphate, triphosphate, C₂₋₆ acyl, or a phosphate prodrug moiety,
each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted carboxylate, or optionally substituted heteroaryloxy.

15. The compound of claim 13, or a pharmaceutically acceptable salt thereof, comprising a structure according to the following formula:

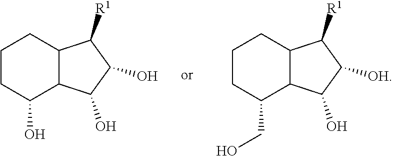

16. The compound of claim 1, or a pharmaceutically acceptable salt thereof, wherein R¹ is selected from the group consisting of adenine, guanine, cytosine, N⁴-hydroxycytosine, thymine, uracil, pyrrolo[2,1-f][1,2,4]triazin-4-amine, and 1,2,4-triazole-3-carboxamide; or
R¹ is 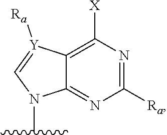
wherein each of X and Rₐ is, independently, H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ,
wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy; and Y is N or C.

17-19. (canceled)

20. The compound of claim 16, wherein R¹ is:

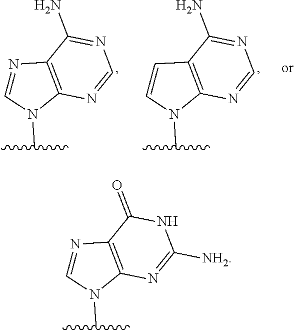

21. The compound of claim 1, wherein R¹ is 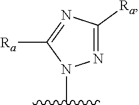
wherein each of Rₐ is, independently, H, deuterium, hydroxyl, Cl, F, CN, Me, Et, (C₁₋₆)OH, ORₓ, (C₁₋₆)COORₓ, CON(Rₓ)₂, (C₁₋₆)CON(Rₓ)₂, N(Rₓ)₂, or Rₓ,
wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, optionally substituted carboxyl, or optionally substituted heteroaryloxy.

22. (canceled)

23. The compound of claim 1, wherein R¹ is 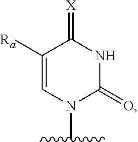
wherein Rₐ is H, deuterium, hydroxyl, Cl, F, CN, Me, Et, (C₁₋₆)OH, ORₓ, (C₁₋₆)COORₓ, (C₁₋₆)CON(Rₓ)₂, N(Rₓ)₂, or Rₓ; and
X is O, NRₓ, or NORₓ,
wherein each of Rₓ is, independently, H, hydroxyl, amino, halo, optionally substituted alkyl, optionally substituted cycloalkyl, optionally substituted aryl, optionally substituted heteroalkyl, optionally substituted heterocycloalkyl, optionally substituted heteroaryl, optionally substituted alkoxy, optionally substituted cycloalkoxy, optionally substituted aryloxy, optionally substituted heteroalkoxy, optionally substituted heterocycloalkoxy, or optionally substituted heteroaryloxy.

24. (canceled)

25. The compound of claim 1, or a pharmaceutically acceptable salt thereof, wherein Pₓ is a phosphate prodrug moiety.

26. (canceled)

27. A compound comprising one of the following formulas, or a pharmaceutically acceptable salt thereof:

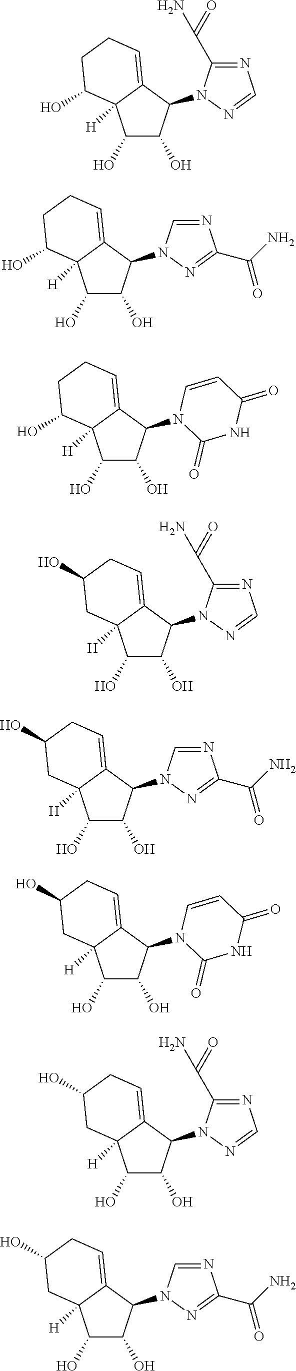

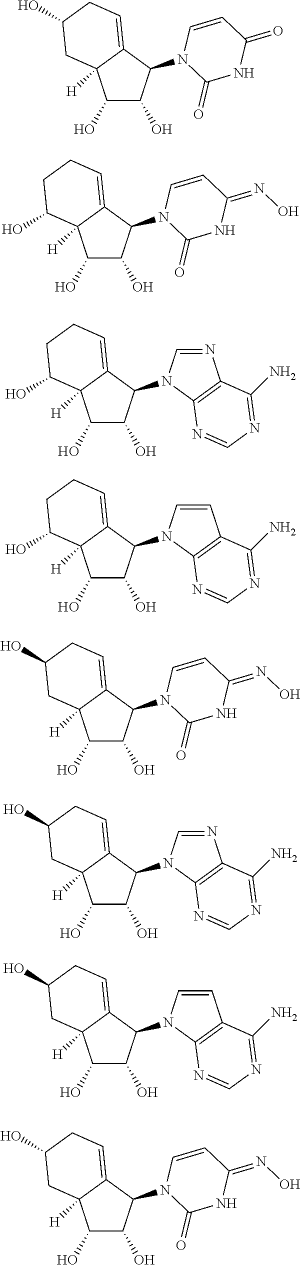

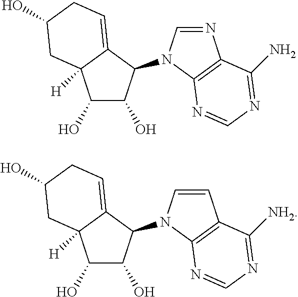

28. A compound comprising one of the following formulas, or a pharmaceutically acceptable salt thereof:

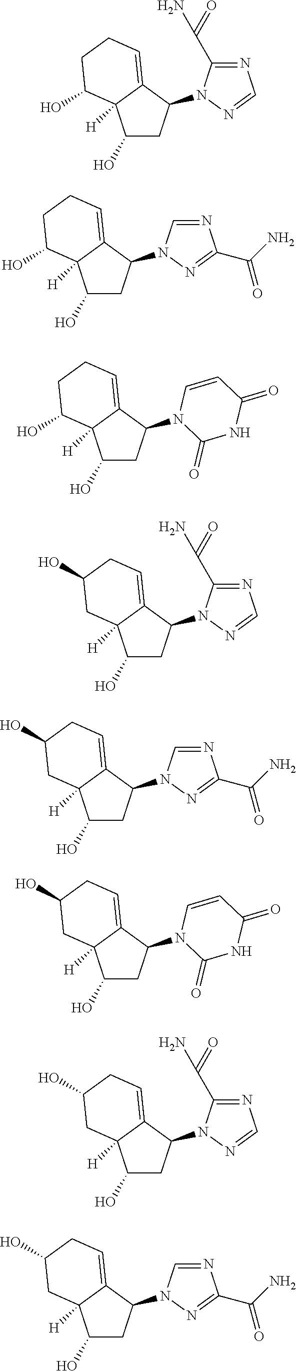

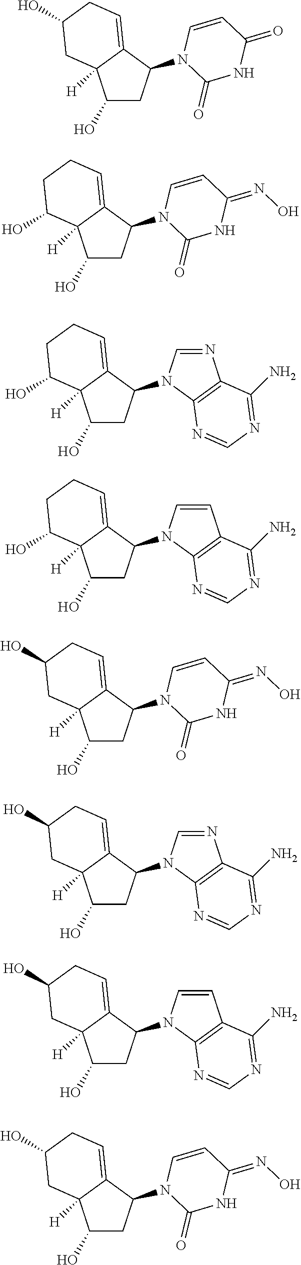

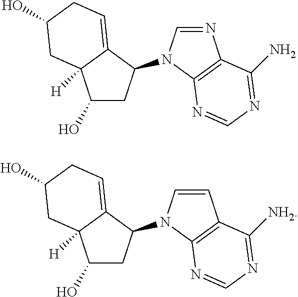

29. The compound of claim 28, or a pharmaceutically acceptable salt thereof, comprising the formula:

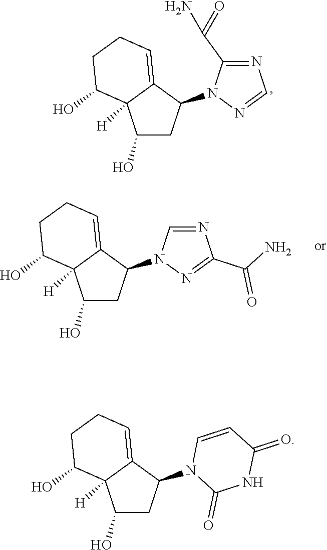 [SMILES: NC(=O)c1ncnn1[C@H]1C[C@H](O)[C@@H]2C1=CCC[C@H]2O ; NC(=O)c1ncn([C@H]2C[C@H](O)[C@@H]3C2=CCC[C@H]3O)n1 ; "or" ; O=c1ccn([C@H]2C[C@H](O)[C@@H]3C2=CCC[C@H]3O)c(=O)[nH]1]

30. (canceled)

31. (canceled)

32. A pharmaceutical composition comprising a compound according to claim 1, or a pharmaceutically acceptable salt thereof, and pharmaceutically acceptable excipient.

33. A method for treating a viral infection, the method comprising administering to a subject in need thereof a therapeutically effective amount of a compound according to claim 1, or a pharmaceutically acceptable salt thereof.

34-40. (canceled)
